SEQUENCE LISTING (16 sequences)

SEQ ID NO: 1 - DNA, 6532 bp, Homo sapiens
  source 1..6532: mol_type = genomic DNA
attgattcccgactgaaggtagagaaggctacgtggtgggggagggtggggggagggtcgcggccgcactggcagtctccgggtgtccggccgtgtcccgaggaagtgcaagacccggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcagaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 2 - protein, 931 aa, Homo sapiens
MAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 3 - DNA, 6323 bp, Homo sapiens
  source 1..6323: mol_type = genomic DNA
tgatgtcaccaatctgcgaccagaccattgattcccgactgaaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 4 - DNA, 6663 bp, Homo sapiens
  source 1..6663: mol_type = genomic DNA
cctggccagcatggtgaaaccccatctctactaaaaatacaaaaattagctgggtgtggtggcgtgcgcctgtaatcccagctactccggaggctgaggcaggagaatcacttgaacccgggaggcggagattgcagtgagctgagatcacactgcactccagcctgattgcagtgagccgagatcatgccactgcactccagcttggcaacagagcgagactccgtctcacaagaaaaaaaataaccgggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcagaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 5 - DNA, 6474 bp, Homo sapiens
  source 1..6474: mol_type = genomic DNA
gaaccggagccatcttgggcccggcgcgcagacccgcggagtttcccgtgccgacgccccggggccacttccagtgcggagtagcggaggcgtgggggcctcgaggggctggcgcggcccagcggtcgggccagggtcgtgccgccggcgggtcgggccgggcaatgcctcgcgggcgcaatgaatccgcggcaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 6 - DNA, 6462 bp, Homo sapiens
  source 1..6462: mol_type = genomic DNA
ggcgcggagagagggaggaccgggcgtcatgctgtttctggcctgaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcagaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 7 - DNA, 6610 bp, Homo sapiens
  source 1..6610: mol_type = genomic DNA
gaaccggagccatcttgggcccggcgcgcagacccgcggagtttcccgtgccgacgccccggggccacttccagtgcggagtagcggaggcgtgggggcctcgaggggctggcgcggcccagcggtcgggccagggtcgtgccgccggcgggtcgggccgggcaatgcctcgcgggcgcaatgaatccgcggcaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcagaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 8 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 9 - DNA, 6532 bp, Homo sapiens
  source 1..6532: mol_type = genomic DNA
gaaccggagccatcttgggcccggcgcgcagacccgcggagtttcccgtgccgacgccccggggccacttccagtgcggagtagcggaggcgtgggggcctcgaggggctggcgcggcccagcggtcgggccagggtcgtgccgccggcgggtcgggccgggcaatgcctcgcgggcgcaatgaatccgcggcaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcagaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagagctccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 10 - protein, 1200 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTELPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 11 - DNA, 6475 bp, Homo sapiens
  source 1..6475: mol_type = genomic DNA
gaaccggagccatcttgggcccggcgcgcagacccgcggagtttcccgtgccgacgccccggggccacttccagtgcggagtagcggaggcgtgggggcctcgaggggctggcgcggcccagcggtcgggccagggtcgtgccgccggcgggtcgggccgggcaatgcctcgcgggcgcaatgaatccgcggcaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcagaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagagctccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 12 - protein, 1181 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQVRKIGELVRYLNTNPVGGLLEYARSHGFAAEFKLVDQSGPPHEPKFVYQAKVGGRWFPAVCAHSKKQGKQEAADAALRVLIGENEKAERMGFTELPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 13 - DNA, 6459 bp, Homo sapiens
  source 1..6459: mol_type = genomic DNA
tgatgtcaccaatctgcgaccagaccattgattcccgactgaaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcagaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 14 - protein, 1235 aa, Homo sapiens
MSPICDQTIDSRLKGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 15 - DNA, 6396 bp, Homo sapiens
  source 1..6396: mol_type = genomic DNA
gaaccggagccatcttgggcccggcgcgcagacccgcggagtttcccgtgccgacgccccggggccacttccagtgcggagtagcggaggcgtgggggcctcgaggggctggcgcggcccagcggtcgggccagggtcgtgccgccggcgggtcgggccgggcaatgcctcgcgggcgcaatgaatccgcggcaggggtattccctcagcggatactacacccatccatttcaaggctatgagcacagacagctcaggtaccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagagctccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatagtatgctccagtgacagatggattagggtgtgtcatactagggtgtgagagaggtaggtcgtagcattcctcatcacatggtcaggggatttttttttctcctttttttttctttttaagccataattggtgatactgaaaactttgggttcccatttatcctgctttctttgggattgctaggcaaggtctggccaggccccccttttttcccccaagtgaagaggcagaaacctaagaagttatcttttctttctacccaaagcatacatagtcactgagcacctgcggtccatttcctcttaaaagttttgttttgatttgtttccatttcctttccctttgtgtttgctacactgacctcttgcggtcttgattaggtttcagtcaactctggatcatgtcagggactgataatttcatttgtggattacgcagacccctctacttcccctctttcccttctgagattctttccttgtgatctgaatgtctccttttccccctcagagggcaaagaggtgaacataaaggatttggtgaaacatttgtaagggtaggagttgaaaactgcagttcccagtgccacggaagtgtgattggagcctgcagataatgcccagccatcctcccatcctgcactttagccagctgcagggcgggcaaggcaaggaaagctgcttccctggaagtgtatcactttctccggcagctgggaagtctagaaccagccagactgggttaagggagctgctcaagcaatagcagaggtttcacccggcaggatgacacagaccacttcccagggagcacgggcatgccttggaatattgccaagcttccagctgcctcttctcctaaagcattcctaggaatattttccccgccaatgctgggcgtacaccctagccaacgggacaaatcctagagggtataaaatcatctctgctcagataatcatgacttagcaagaataagggcaaaaaatcctgttggcttaacgtcactgttccacccggtgtaatatctctcatgacagtgacaccaagggaagttgactaagtcacatgtaaattaggagtgttttaaagaatgccatagatgttgattcttaactgctacagataacctgtaattgagcagatttaaaattcaggcatacttttccatttatccaagtgctttcatttttccagatggcttcagaagtaggctcgtgggcagggcgcagacctgatctttatagggttgacatagaaagcagtagttgtgggtgaaagggcaggttgtcttcaaactctgtgaggtagaatcctttgtctatacctccatgaacattgactcgtgtgttcagagcctttggcctctctgtggagtctggctctctggctcctgtgcattctttgaatagtcactcgtaaaaactgtcagtgcttgaaactgtttcctttactcatgttgaagggactttgttggcttttagagtgttggtcatgactccaagagcagagcagggaagagcccaagcatagacttggtgccgtggtgatggctgcagtccagttttgtgatgctgcttttacgtgtccctcgataacagtcagctagacacactcaggaggactactgaggctctgcgaccttcaggagctgagcctgcctctctcctttagatgacagaccttcatctgggaacgtgctgagccagcaccctcagatgatttccctccaaactgctgactaggtcatcctctgtctggtagagacattcacatctttgcttttattctatgctctctgtacttttgaccaaaaattgaccaaagtaagaaaatgcaagttctaaaaatagactaaggatgcctttgcagaacaccaaagcatcccaaggaactggtagggaagtggcgcctgtctcctggagtggaagaggcctgctccctggctctgggtctgctgggggcacagtaaatcagtcttggcacccacatccagggcagagaggtctgtggttctcagcatcagaaggcagcgcagcccctctcctcttcaggctacagggttgtcacctgctgagtcctcaggttgtttggcctctctggtccatcttgggcattaggttctccagcagagctctggccagctgcctcttctttaactgggaacacaggctctcacaagatcagaacccccactcacccccaagatcttatctagcaagcctgtagtattcagtttctgttgtaggaagagagcgaggcatccctgaattccacgcatctgctggaaacgagccgtgtcagatcgcacatccctgcgcccccatgcccctctgagtcacacaggacagaggaggcagagcttctgcccactgttatcttcactttctttgtccagtcttttgtttttaataagcagtgaccctccctactcttctttttaatgatttttgtagttgatttgtctgaactgtggctactgtgcattccttgaataatcacttgtaaaaattgtcagtgcttgaagctgtttcctttactcacattgaagggacttcgttggttttttggagtcttggttgtgactccaagagcagagtgaggaagacccccaagcatagactcgggtactgtgatgatggctgcagtccagttttatgattctgcttttatgtgtcccttgataacagtgacttaacaatatacattcctcataaataaaaaaaaaacaagaatctgaattcttagaaa

SEQ ID NO: 16 - protein, 905 aa, Homo sapiens
MAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTELPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

(19) United States
(12) Patent Application Publication
(10) Pub. No.: US 2026/0265832 A1
(43) Pub. Date: Sep. 10, 2026

(54) ADAR1 INHIBITORS FOR TREATING CANCER

(71) Applicants: INSTITUE OF CANCER RESEARCH ROYAL CANCER HOSPITAL, London (GB); INSTITUT GUSTAVE ROUSSY, Villejuif (FR); UNIVERSITÉ PARIS-SACLAY, Gif-sur-Yvette (FR); INSTITUT NATIONAL DE LA SANTÉ ET DE LA RECHERCHE MÉDICALE (INSERM), Paris (FR)

(72) Inventors: Christopher LORD, London (GB); Roman CHABANON, Villejuif (FR); Sophie POSTEL-VINAY, Villejuif (FR)

(73) Assignees: INSTITUE OF CANCER RESEARCH ROYAL CANCER HOSPITAL, London (GB); INSTITUT GUSTAVE ROUSSY, Villejuif (FR); UNIVERSITÉ PARIS-SACLAY, Gif-sur-Yvette (FR); INSTITUT NATIONAL DE LA SANTÉ ET DE LA RECHERCHE MÉDICALE (INSERM), Paris (FR)

(21) Appl. No.: 19/165,297

(22) PCT Filed: Mar. 15, 2024
(86) PCT No.: PCT/IB2024/000134

§ 371 Date: Sep. 15, 2025

(30) Foreign Application Priority Data
Mar. 15, 2023 (GB) 2303784.9

(51) Int. Cl. C12Q 1/6886; A61K 31/365; A61K 31/7068; A61K 45/06; C12Q 1/686; G01N 33/575

(52) U.S. Cl. CPC C12Q 1/6886; A61K 31/365; A61K 31/7068; A61K 45/06; C12Q 1/686; G01N 33/57585; C12Q 2600/106; C12Q 2600/156

(57) ABSTRACT

The present invention relates to adenosine deaminase 1 (ADAR1) inhibitors for use in a method of treating an individual with a homologous recombination defective (HRD) cancer, as well as methods for selecting individuals suitable for such treatments.

### FIELD OF THE INVENTION

The present invention relates to adenosine deaminase 1 (ADAR1) inhibitors for use in a method of treating an individual with a homologous recombination defective (HRD) cancer, as well as methods for selecting individuals suitable for such treatments.

### BACKGROUND TO THE INVENTION

The homologous recombination (HR) pathway is essential for high-fidelity DNA double strand break (DSB) repair and involves numerous genes including BRCA1 and BRCA2. HR deficiency due to inactivation of such genes leads to impaired DSB repair and increased levels of genomic alterations. As a result, HR deficiency is a frequent driver of tumorigenesis, and is commonly observed in cancers such as breast, ovarian, prostate and pancreatic cancers.

Poly ADP-ribose polymerase (PARP) inhibitors were the first clinically approved drugs for use in treating homologous recombination defective (HRD) cancers that were developed based on the genetic concept of synthetic lethality. This concept describes a genetic interaction whereby a defect in either one of two genes does not affect cell fitness but a combination of defects in both genes results in cell death. In other words, the use of PARP inhibitors results in the selective death of cells with an HR defect.

Under normal conditions, DNA damage results in activation of the PARP enzymes, notably PARP1. ADP-ribose units are recruited to sites of DNA breaks by PARP1, in a process known as PARylation. With the assistance of PARP and other DNA repair enzymes, repair of DNA breaks occurs, allowing normal cells to survive DNA damage. In HRD cancer cells, PARP inhibitors mediate inhibition of PARylation and PARP1 trapping on DNA, thereby causing DSBs that accumulate in the absence of HR-mediated repair. This synthetic lethality ultimately leads to cell death.

PARP inhibitors have shown promising outcomes since their clinical applications have been approved as maintenance treatment for patients with BRCA-mutant cancers. However, whilst PARP inhibitors can achieve therapeutic effects, the development of secondary resistance to treatment is a significant issue, affecting virtually all patients in the advanced/metastatic setting. Other limitations of PARP inhibitors include dose-limiting toxicities such as frequent haematological toxicities, gastrointestinal adverse events, renal toxicity, liver toxicity and fatigue—each of which can lead to treatment discontinuation. Whilst the frequency of discontinuation can vary between different PARP inhibitors, ^{~}10-15% discontinuation has been reported across a range of PARP inhibitors (LaFargue et al. The Lancet (2019) 20(1):e15-e28).

Furthermore, it has proven very difficult to combine PARP inhibitors with standard-of-care chemotherapeutic regimens due to overlapping toxicity profiles. This is for example in the case in ovarian cancer, where PARP inhibitors are given as a maintenance treatment following standard-of-care platinum-based induction chemotherapy, as opposed to being used simultaneously with platinum-based chemotherapy.

Therefore, there is an ongoing need to develop new cancer drugs as alternative or complementary treatments for HRD cancers, which have the potential to overcome one or more of the limitations of PARP inhibitors. It is an object of the present invention to address one or more of these issues.

### SUMMARY OF THE INVENTION

At present, there remains a pressing need for new and effective treatments for homologous recombination defective (HRD) cancers, and particularly for effective treatments which overcome one or more of the limitations associated with PARP inhibitors, including the development of resistance. To date, research addressing the development of PARP inhibitor resistance has focused on combining PARP inhibitors with other anticancer therapies, such as DNA-damaging agents, immune-checkpoint inhibitors, anti-angiogenic agents, PI3K inhibitors and other targeted therapies. The combination of PARP inhibitors with agents that inhibit the activity of the error-prone DNA polymerase theta (PolQ or Pole) or ATR are currently a particular focus. However, these approaches are all centred on HRD/PARP synthetic lethality and checkpoint maintenance, rather than identifying new targets/mechanisms for the treatment of HRD cancer. As such, these conventional approaches differ from the present invention.

The present inventors have demonstrated for the first time that inhibition of ADAR1 results in synthetic lethality in HRD cells. In particular, the present inventors have carried out an unbiased medium-throughput RNA interference screen and identified that suppression of ADAR1 gene significantly reduces viability of cells harbouring a BRCA1 loss-of-function mutation, but not cells comprising a functional BRCA1 gene. The inventors have further shown that this BRCA1/ADAR1 synthetic lethality can be elicited by numerous approaches, including using CRISPR-Cas9-mediated genetic knockout, siRNA-mediated transcriptional silencing, and small molecule ADAR1 inhibitors. Furthermore, the present inventors have demonstrated that this synthetic lethality extends to HRD associated with mutations/deficiencies in other genes. Notably, BRCA2/ADAR1 synthetic lethality has been demonstrated, again using a range of modalities for ADAR1 inhibition (CRISPR-Cas9-mediated genetic knockout and small molecule ADAR1 inhibitors). The inventors have further elucidated the mechanism underlying this HDR/ADAR1 synthetic lethality, namely that ADAR1 inhibition causes an accumulation of DNA damage and selective genomic instability in HRD cancer cells, and that this synthetic lethality extends across cell lines from multiple cancer types (including breast cancer, colorectal cancer and other non-breast cancer cell lines). In particular, as demonstrated herein, ADAR1 inhibition increases replication stress and R-loop burden in cells harbouring a BRCA1 loss-of-function mutation, resulting in activation of the replication stress response and apoptosis. Significantly, the present inventors have shown that this ADAR1/HR deficiency synthetic lethality is maintained when PARP inhibitor resistance occurs, suggesting that the present invention has potential utility in treating patients who have developed resistance to PARP inhibitors.

Accordingly, the present invention provides an adenosine deaminase 1 (ADAR1) inhibitor for use in a method of treating an individual with a homologous recombination defective (HRD) cancer.

The homologous recombination deficiency may be associated with a mutation and/or deficiency in one or more gene associated with HRD, optionally wherein said one or more gene associated with HRD is selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ. The homologous recombination deficiency may be associated with a mutation and/or deficiency in BRCA1 and/or BRCA2, preferably BRCA1.

The cancer may be breast cancer, ovarian cancer, pancreatic cancer, biliary tract cancer or prostate cancer. The cancer may be: (a) BRCA-mutated; (b) PARP inhibitor sensitive; and/or (c) BRCA-mutated and HER2-negative breast cancer.

The ADAR1 inhibitor may be for use in a method of treating an individual with an HRD cancer, said method comprising determining in a sample obtained from the individual whether the cancer is an HRD cancer; and optionally administering a therapeutically effective amount of an ADAR1 inhibitor to the individual with HRD cancer.

Determining whether the cancer is an HRD cancer may comprise: (a) determining the presence of a deficiency (e.g. epigenetic silencing) and/or mutation in one or more gene associated with homologous recombination deficiency, optionally a deficiency and/or mutation in one or more gene selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ; (b) use of a companion diagnostic for homologous recombination deficiency, optionally which determines and/or quantifies loss of heterozygosity (LOH), telomeric allelic imbalance (LAI) and/or large-scale state transitions (LST), or any combination thereof; (c) detecting and/or quantifying RAD51 foci within the sample, wherein reduced RAD51 signal is associated with homologous recombination deficiency; (d) detecting and/or quantifying a homologous recombination deficiency defect, optionally a transcriptomic signature and/or mutational scar associated with homologous recombination deficiency; and/or (e) detecting and/or quantifying platinum sensitivity of the cancer, wherein platinum sensitivity is associated with homologous recombination deficiency.

The step of determining the presence of a deficiency (e.g. epigenetic silencing) and/or mutation in one or more gene associated with homologous recombination deficiency may be performed on nucleic acid sequences obtained from an individual's cancerous or noncancerous cells, optionally using direct sequencing, hybridisation to a probe, restriction fragment length polymorphism (RFLP) analysis, single-stranded conformation polymorphism (SSCP), PCR amplification of specific alleles, amplification of DNA target by PCR followed by a mini-sequencing assay, allelic discrimination during PCR, Genetic Bit Analysis, pyrosequencing, oligonucleotide ligation assay, analysis of melting curves, testing for a loss of heterozygosity (LOH) or next generation sequencing (NGS) techniques, single molecule sequencing techniques or nano string nCounter technology.

The step of determining the presence of a deficiency (e.g. epigenetic silencing) and/or mutation in one or more gene associated with homologous recombination deficiency may comprise or consist of measuring protein expression of the one or more homologous recombination deficiency-associated gene in a sample obtained from the individual to determine whether the protein is mutated or deficient, wherein optionally the step of determining protein expression of the one or more homologous recombination deficiency-associated gene comprises determining protein expression of the one or more homologous recombination deficiency-associated gene in the sample using one or more of immunohistochemistry, determining protein levels in a cell lysate by ELISA or Western blotting, and/or determining protein expression using a binding agent capable of specifically binding to a protein, or a fragment thereof.

The step of determining the expression of the one or more gene associated with homologous recombination deficiency may comprise or consist of extracting RNA from a sample of cancer cells and measuring expression by real time PCR and/or by using a probe capable of hybridising to the RNA of one or more gene associated with homologous recombination deficiency. The probe may be immobilised in a microarray.

The step of determining whether the individual has a cancer deficient in the one or more gene associated with homologous recombination deficiency may comprise or consist of identifying gene loss resulting from chromosomal instability through karyotype analysis of a sample obtained from the individual.

The step of determining whether the individual has a cancer deficient in the one or more gene associated with homologous recombination deficiency may comprise or consist of identifying gene loss or pathogenic loss-of-function mutation detected in the individual's circulating tumour DNA or cell-free nucleic acids.

The ADAR1 inhibitor may be a small molecule, a proteolysis-targeting chimeric molecule (PROTAC), a macrocyclic molecule, a molecular glue, a nucleic acid inhibitor, an antibody, an antibody-drug conjugate and/or a peptide. The ADAR1 inhibitor may be a small molecule selected from 8-azaadenosine, 8-chloroadenosine, 8-azanebularine, AVA-ADR-001, ZYS-1, 8-azanebularine, AVA-ADR-001 and rebecsinib.

Treatment with an ADAR1 inhibitor may be combined with one or more further anti-cancer therapies. Treatment with an ADAR1 inhibitor may be used in conjunction with one or more further chemotherapeutic agent(s), targeted therapy, or antibody-drug conjugate. Alternatively or additionally, treatment with an ADAR1 inhibitor may be used in conjunction with one or more immunotherapeutic agent, which is optionally selected from an immune checkpoint inhibitor, a monoclonal antibody (including bi-, tris- or multi-specific antibodies), or a cytokine, a cell therapy, an oncolytic virus, a cancer vaccine, an antisense oligodeoxynucleotide, and/or an agonist of a nucleic acid sensing pathway (e.g. agonist of the cGAS/stimulator of interferon genes (STING) pathway, i.e. a STING agonist). Further alternatively or additionally, treatment with an ADAR1 inhibitor may be used in conjunction with radiotherapy.

The invention further provides a method of selecting an individual having cancer for treatment with an adenosine deaminase 1 (ADAR1) inhibitor, the method comprising: (a) determining in a sample obtained from the individual whether the cancer is an HRD cancer; (b) selecting the individual for treatment with the ADAR1 inhibitor where the cancer is an HRD cancer; and (c) providing an ADAR1 inhibitor suitable for administration to the individual. Said method may further comprise administering a therapeutically effective amount of the ADAR1 inhibitor to the individual.

Determining whether the cancer is an HRD cancer may comprise: (a) determining the presence of a deficiency (e.g. epigenetic silencing) and/or mutation in one or more gene associated with homologous recombination deficiency, optionally a deficiency and/or mutation in one or more gene selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ; (b) use of a companion diagnostic for homologous recombination deficiency, optionally which determines and/or quantifies loss of heterozygosity (LOH), telomeric allelic imbalance (LAI) and/or large-scale state transitions (LST), or any combination thereof; (c) detecting and/or quantifying RAD51 foci within the sample, wherein reduced RAD51 signal is associated with homologous recombination deficiency; (d) detecting and/or quantifying a homologous recombination deficiency defect, optionally a transcriptomic signature and/or mutational scar associated with homologous recombination deficiency; and/or (e) detecting and/or quantifying platinum sensitivity of the cancer, wherein platinum sensitivity is associated with homologous recombination deficiency. Preferably the one or more gene associated with homologous recombination deficiency is BRCA1 and/or BRCA2, more preferably BRCA1.

The cancer may be breast cancer, ovarian cancer, pancreatic cancer, biliary tract cancer or prostate cancer. The cancer may be: (a) BRCA-mutated; (b) PARP inhibitor sensitive; and/or (c) BRCA-mutated and HER2-negative breast cancer.

The step of determining the presence of a deficiency (e.g. epigenetic silencing) and/or mutation in one or more gene associated with homologous recombination deficiency may be performed on nucleic acid sequences obtained from an individual's cancerous or noncancerous cells, optionally using direct sequencing, hybridisation to a probe, restriction fragment length polymorphism (RFLP) analysis, single-stranded conformation polymorphism (SSCP), PCR amplification of specific alleles, amplification of DNA target by PCR followed by a mini-sequencing assay, allelic discrimination during PCR, Genetic Bit Analysis, pyrosequencing, oligonucleotide ligation assay, analysis of melting curves, testing for a loss of heterozygosity (LOH) or next generation sequencing (NGS) techniques, single molecule sequencing techniques or nanostring nCounter technology.

The step of determining the presence of a deficiency (e.g. epigenetic silencing) and/or mutation in one or more gene associated with homologous recombination deficiency may comprise or consist of measuring protein expression of the one or more homologous recombination deficiency-associated gene in a sample obtained from the individual to determine whether the protein is mutated or deficient, wherein optionally the step of determining protein expression of the one or more homologous recombination deficiency-associated gene comprises determining protein expression of the one or more homologous recombination deficiency-associated gene in the sample using one or more of immunohistochemistry, determining protein levels in a cell lysate by ELISA or Western blotting, and/or determining protein expression using a binding agent capable of specifically binding to a protein, or a fragment thereof.

The step of determining the expression of the one or more gene associated with homologous recombination deficiency may comprise or consist of extracting RNA from a sample of cancer cells and measuring expression by real time PCR and/or by using a probe capable of hybridising to the RNA of one or more gene associated with homologous recombination deficiency. The probe may be immobilised in a microarray.

The step of determining whether the individual has a cancer deficient in the one or more gene associated with homologous recombination deficiency may comprise or consist of identifying gene loss resulting from chromosomal instability through karyotype analysis of a sample obtained from the individual.

The step of determining whether the individual has a cancer deficient in the one or more gene associated with homologous recombination deficiency may comprise or consist of identifying gene loss or pathogenic loss-of-function mutation detected in the individual's circulating tumour DNA or cell-free nucleic acids.

The ADAR1 inhibitor may be a small molecule, a proteolysis-targeting chimeric molecule (PROTAC), a macrocyclic molecule, a molecular glue, a nucleic acid inhibitor, an antibody, an antibody-drug conjugate and/or a peptide. The ADAR1 inhibitor may be a small molecule selected from 8-azaadenosine, 8-chloroadenosine, ZYS-1, 8-azanebularine, AVA-ADR-001 and rebecsinib.

Treatment with an ADAR1 inhibitor may be combined with one or more further anti-cancer therapies. Treatment with an ADAR1 inhibitor may be used in conjunction with one or more further chemotherapeutic agent(s). Alternatively or additionally, treatment with an ADAR1 inhibitor may be used in conjunction with one or more immunotherapeutic agent, which is optionally selected from an immune checkpoint inhibitor, a monoclonal antibody (including bi-, tri- or multi-specific antibodies), a cytokine, a cell therapy, an oncolytic virus, a cancer vaccine, an antisense oligodeoxynucleotide, and/or an agonist of the nucleic acid sensing pathway (e.g. agonist of the cGAS/stimulator of interferon genes (STING) pathway, i.e. a STING agonist). Further alternatively or additionally, treatment with an ADAR1 inhibitor may be used in conjunction with radiotherapy.

### BRIEF DESCRIPTION OF THE DRAWINGS

FIG. 1. A. An unbiased RNA interference screen identifies ADAR1-BRCA1 synthetic lethality. BRCA1-mutant tumour cells (SUM149 BRCA1-Mut) and a BRCA1 functional daughter clone with a BRCA1 reversion mutation (SUM149 BRCA1-Rev) were reverse transfected with a library of siRNAs arrayed in 96 well plates. The library included siRNAs designed to target one of a series of proteins involved in pattern receptor proteins involved in innate immunity. After six subsequent days of continuous culture, cell viability was determined by use of Cell TitreGlo reagent. B. Results from the screen displayed as Surviving Fraction (SF) elicited by each siRNA, compared to cells transfected with control, non-targeting, siRNAs (siCTRL #1, #2, #3). As a control, cells transfected with siRNA designed to target the mitotic checkpoint kinase PLK1 caused a profound reduction in SF in both SUM149 BRCA1-Mut and SUM149 BRCA1-Rev cells compared to non-targeting control transfected cells. From this screen, two genes, when targeted by siRNA elicited differential effects in SUM149 BRCA1-Mut vs. SUM149 BRCA1-Rev cells; ADAR1 and STING. Of these, siRNA targeting of ADAR1 caused a profound reduction in the SF of SUM149 BRCA1-Mut cells (SF of 0.2, i.e. 80% reduction in SF), whilst having minimal effects in SUM149 BRCA1-Rev cells (SF of 0.95).

FIG. 2. A. Western blot of SUM149 BRCA1-Mut and SUM149 BRCA1-Rev cells transfected with ADAR1 sgRNA. Cells were either transfected with control, non-targeting, sgRNA (sgCTRL), or transfected with ADAR1 sgRNAs (#1, #2, #3, #4) in the presence of Edit-R Cas9 recombinase as shown. 48 hours after transfection, cell lysates were generated and western blotted to detect ADAR1 protein. B, C. Clonogenic survival of SUM149 BRCA1-Mut and SUM149 BRCA1-Rev cells transfected with ADAR1 sgRNA as described in A. After transfection, cells were continuously cultured for 8 days, after which colonies were stained and counted. D, E, F. Incucyte cell growth assays of SUM149 BRCA1-Mut and SUM149 BRCA1-Rev cells transfected with ADAR1 sgRNA as described in A. After transfection, cells were continuously cultured for 6 days, during which relative confluency was monitored by use of an Incucyte. After this time, cell viability was determined by CellTiter-Glo® (F). G. Western blot of RPE1 BRCA1-wildtype and RPE1 BRCA1-knockout cells transfected with ADAR1 sgRNA. Cells were either transfected with control, non-targeting, sgRNA (sgCTRL), or transfected with ADAR1 sgRNAs (#1, #2, #3, #4) in the presence of Edit-R Cas9 recombinase as shown. 48 hours after transfected cell lysates were generated and western blotted to detect ADAR1 protein. H, I. Clonogenic survival of RPE1 BRCA1-wildtype and RPE1 BRCA1-knockout cells transfected with ADAR1 sgRNA as described in A. After transfection, cells were continuously cultured for 14 days, after which colonies were stained and counted. J, K, L. Incucyte cell growth assays of RPE1 BRCA1-wildtype and RPE1 BRCA1-knockout cells transfected with ADAR1 sgRNA as described in A. After transfection, cells were continuously cultured for 8 days, during which relative confluency was monitored by use of an Incucyte. After this time, cell viability was determined by CellTiter-Glo® (L). All graphs show median and individual data point. Error bars represent Standard Deviation (SD) or the min to max range in case of box-and-whiskers plots. p values are as follows: ns=not significant, * p<0.05, ** p<0.005, *** p<0.0005, **** p<0.00005.

FIG. 3. A. Western blot of SUM149 BRCA1-Mut and SUM149 BRCA1-Rev cells transfected with ADAR1 siRNA. Cells were either transfected with control non-targeting siRNA (siCTRL) or transfected with a titration of ADAR1 siRNA SMARTpool. 72 hours after transfection, cell lysates were generated and western blotted to detect ADAR1 protein. B. Quantification of cell survival after siRNA transfection. SUM149 BRCA1-Mut and SUM149 BRCA1-Rev cells were transfected with ADAR1 siRNA as described in A. After transfection, cells were continuously cultured for 6 days, after which cell viability was determined by CellTiter-Glo®. C. Western blot of SUM149 BRCA1-Mut and SUM149 BRCA1-Rev cells transfected with ADAR1 siRNA. Cells were transfected with control, non-targeting, SIRNA (siCTRL), or transfected with either ADAR1 siRNA SMARTpool (P) or two individual ADAR1 siRNAs (#1, #2). 72 hours after transfection, cell lysates were generated and western blotted to detect ADAR1 protein. Short (SE) and long (LE) exposure times were used to image the membranes. D, E. Clonogenic survival of SUM149 BRCA1-Mut and SUM149 BRCA1-Rev cells transfected with ADAR1 siRNA as described in C. After transfection, cells were continuously cultured for 10 days, after which colonies were stained and counted. F. Western blot of BRCA1-wildtype (MDA-MB-231, Hs578T, CAL51, CAL120) or BRCA1-mutant (MDA-MB-436, HCC1937) cells transfected with ADAR1 siRNA. Cells were either transfected with control, non-targeting, siRNA (siCTRL) or transfected with a titration of ADAR1 siRNA SMARTpool. 72 hours after transfection, cell lysates were generated and western blotted to detect ADAR1 protein. G. Quantification of cell survival after siRNA transfection. Cells were transfected with ADAR1 siRNA as described in F. After transfection, cells were continuously cultured for 6 days, after which cell viability was determined by CellTiter-Glo®. The cell viability of SUM149 BRCA1-Mut and SUM149 BRCA1-Rev cells in the same conditions is showed as an indication. H. Western blot of Brca1-wildtype and Brca1-mutant (Δ11) mouse embryonic fibroblasts (MEFs) transfected with Adar1 siRNA. Cells were either transfected with control, non-targeting, siRNA (siCTRL), or transfected with a titration of Adar1 siRNA SMARTpool. 72 h hours after transfection, cell lysates were generated and western blotted to detect ADAR1 protein. I, J. Clonogenic survival of Brca1-wildtype and Brca1-mutant (Δ11) MEFs transfected with Adar1 siRNA as described in H. After transfection, cells were continuously cultured for 6 days, after which colonies were stained and counted. All graphs show median and individual data point. Error bars represent Standard Deviation (SD) or the min to max range in case of box-and-whiskers plots. p values are as follows: ns=not significant, * p<0.05, ** p<0.005, *** p<0.0005, **** p<0.00005.

FIG. 4. A. Western blot of DLD1 BRCA2-wildtype and DLD1 BRCA2-knockout cells transfected with ADAR1 sgRNA. Cells were either transfected with control, non-targeting, sgRNA (sgCTRL), or transfected with ADAR1 sgRNA (#1, #2, #3, #4) in the presence of Edit-R Cas9 recombinase as shown. 48 hours after transfection, cell lysates were generated and western blotted to detect ADAR1 protein. B, C. Clonogenic survival of DLD1 BRCA2-wildtype and DLD1 BRCA2-knockout cells transfected with ADAR1 sgRNA as described in A. After transfection, cells were continuously cultured for 14 days, after which colonies were stained and counted. D, E, F. Incucyte cell growth assays of DLD1 BRCA2-wildtype and DLD1 BRCA2-knockout cells transfected with ADAR1 sgRNA as described in A. After transfection, cells were continuously cultured for 9 days, during which relative confluency was monitored by use of an Incucyte. After this time, cell viability was determined by CellTiter-Glo® (F). All graphs show median and individual data point. Error bars represent Standard Deviation (SD) or the min to max range in case of box-and-whiskers plots. p values are as follows: ns=not significant, * p<0.05, ** p<0.005, *** p<0.0005, **** p<0.00005.

FIG. 5. A. Western blot of HEK293T ADAR1-wildtype and HEK293T ADAR1-knockout cells transfected with BRCA1 siRNA. Cells were either transfected with control, non-targeting, siRNA (siCTRL) or transfected with a titration of BRCA1 siRNA SMARTpool. 72 hours after transfection, cell lysates were generated and western blotted to detect ADAR1 and BRCA1 proteins. B. Quantification of cell survival after siRNA transfection. HEK293T ADAR1-wildtype and HEK293T ADAR1-knockout cells were transfected with BRCA1 siRNA as described in A. After transfection, cells were continuously cultured for 6 days, after which cell viability was determined by CellTiter-Glo®. C. Western blot of HEK293T ADAR1-wildtype and HEK293T ADAR1-knockout cells transfected with BRCA2 siRNA. Cells were either transfected with control, non-targeting, siRNA (siCTRL) or transfected with a titration of BRCA2 siRNA SMARTpool. 72 hours after transfection, cell lysates were generated and western blotted to detect ADAR1 and BRCA2 proteins. D. Quantification of cell survival after siRNA transfection. HEK293T ADAR1-wildtype and HEK293T ADAR1-knockout cells were transfected with BRCA2 siRNA as described in C. After transfection, cells were continuously cultured for 6 days, after which cell viability was determined by CellTiter-Glo®. All graphs show median and individual data points. Error bars represent Standard Deviation (SD) or the min to max range in case of box-and-whiskers plots. p values are as follows: ns=not significant, * p<0.05, ** p<0.005, *** p<0.0005, **** p<0.00005.

FIG. 6. A. Quantification of cell survival in SUM149 BRCA1-Mut, SUM149 BRCA1-Rev, and two PARP1-altered daughter clones harbouring either a PARP1 null mutation (SUM149 BRCA1-Mut PARP1-KO) or a mutation in PARP1 ZnF domains that abolishes its DNA binding ability (SUM149 BRCA1-Mut PARP1-p.43ΔMFD), transfected with ADAR1 siRNA. Cells were either transfected with control, non-targeting, siRNA (siCTRL) or transfected with a titration of ADAR1 siRNA SMARTpool. After transfection, cells were continuously cultured for 6 days, after which cell viability was determined by CellTiter-Glo®. B. Dose-response survival curves of SUM149 BRCA1-Mut, SUM149 BRCA1-Rev, SUM149 BRCA1-Mut PARP1-KO, and SUM149 BRCA1-Mut PARP1-p.43ΔMFD exposed to increasing concentrations of the PARP inhibitor talazoparib for 7 days. All graphs show median and individual data points. Error bars represent Standard Deviation (SD) or the min to max range in case of box-and-whiskers plots. p values are as follows: ns=not significant, * p<0.05, ** p<0.005, *** p<0.0005, **** p<0.00005.

FIG. 7. A. Clonogenic survival of Brca1-wildtype and Brca1-mutant (Δ11) MEFs exposed to 8-azaadenosine for 8 days. B. Dose-response survival curves of Brca1-wildtype and Brca1-mutant (Δ11) MEFs exposed to increasing concentrations of 8-azaadenosine for 7 days. C. Clonogenic survival of BRCA2-wildtype and BRCA2-knockout DLD1 cells exposed to 8-azaadenosine for 8 days. D. Dose-response survival curves of BRCA2-wildtype and BRCA2-knockout DLD1 cells exposed to increasing concentrations of 8-azaadenosine for 7 days. All graphs show median. Error bars represent Standard Deviation (SD).

FIG. 8. A, B, C. Immunofluorescence detection of γH2AX in SUM149 BRCA1-Mut and BRCA1-Rev cells transfected with ADAR1 siRNA. Cells were transfected with control, non-targeting, siRNA (siCTRL) or transfected with either ADAR1 siRNA SMARTpool (P) or two individual ADAR1 siRNAs (#1, #2). 72 hours after transfection, cells were fixed and imaged (A) to detect γH2AX foci (B, C). Scale bar, 10 μm. D, E. Immunofluorescence detection of γH2AX in Brca1-wildtype and Brca1-mutant (Δ11) mouse embryonic fibroblasts (MEFs) transfected with Adar1 siRNA. Cells were either transfected with control, non-targeting, siRNA (siCTRL) or transfected with Adar1 siRNA SMARTpool. 72 h hours after transfection, cells were fixed and imaged to detect γH2AX foci. F, G. Immunofluorescence detection of γH2AX foci in DLD1 BRCA2-wildtype and BRCA2-knockout cells transfected with ADAR1 siRNA. Cells were transfected with control, non-targeting, siRNA (siCTRL) or transfected with either ADAR1 siRNA SMARTpool (P) or two individual ADAR1 siRNAs (#1, #2). 72 hours after transfection, cells were fixed and imaged to detect γH2AX foci. H, I, J. Immunofluorescence detection of γH2AX and RAD51 in SUM149 BRCA1-Mut and BRCA1-Rev cells transfected with ADAR1 siRNA. Experiment conducted as in A-C. 72 hours after transfection, cells were fixed and imaged (H) to detect RAD51 foci (I, J). Scale bar, 10 μm. K, L. Immunofluorescence detection of micronuclei in SUM149 BRCA1-Mut and BRCA1-Rev cells transfected with ADAR1 siRNA. Experiment conducted as in A-C. 72 hours after transfection, cells were fixed and imaged (K) to detect micronuclei (L). Scale bar, 10 μm. M. Immunofluorescence detection of micronuclei in Brca1-wildtype and Brca1-mutant (11) mouse embryonic fibroblasts (MEFs) transfected with Adar1 siRNA. Experiment conducted as in D-E. 72 hours after transfection, cells were fixed and imaged to detect micronuclei. N. Immunofluorescence detection of micronuclei in DLD1 BRCA2-wildtype and BRCA2-knockout cells transfected with ADAR1 siRNA. Experiment conducted as in F-G. 72 hours after transfection, cells were fixed and imaged to detect micronuclei. All graphs show median and individual data points. Error bars represent Standard Deviation (SD). p values are as follows: ns=not significant, * p<0.05, ** p<0.005, *** p<0.0005, **** p<0.00005.

FIG. 9. A, B, C. Immunofluorescence detection of RPA and CCNA2 in SUM149 BRCA1-Mut and BRCA1-Rev cells transfected with ADAR1 siRNA. Cells were transfected with control, non-targeting, siRNA (siCTRL) or transfected with either ADAR1 siRNA SMARTpool (P) or an individual ADAR1 siRNA (#1). 72 hours after transfection, cells were fixed and imaged (A) to detect RPA foci in S-phase-positive cells (CCNA2-positive; B, C). Scale bar, 10 μm. D, E, F. Immunofluorescence detection of R-loops (S9.6) and nucleoli (fibrillarin) in SUM149 BRCA1-Mut and BRCA1-Rev cells transfected with ADAR1 siRNA. Cells were transfected with control, non-targeting, siRNA (ciCTRL) or transfected with either ADAR1 siRNA SMARTpool (P) or an individual ADAR1 siRNA (#1). 72 hours after transfection, cells were fixed and imaged (D) to detect nuclear S9.6 foci outside nucleoli (fibrillarin-negative; E, F). Exogenous RNase H digestion was used to control for the R-loop specificity of S9.6 staining. Scale bar, 10 μm. G. Western blot of SUM149 BRCA1-Mut and SUM149 BRCA1-Rev cells transfected with ADAR1 siRNA. Cells were either transfected with control, non-targeting, siRNA (siCTRL) or transfected with a titration of ADAR1 siRNA SMARTpool. 72 hours after transfection, cell lysates were generated and western blotted to detect phosphorylated CHK1 (p-CHK1), CHK1, γH2AX, and cleaved PARP1 (c-PARP1) proteins. H. Western blot of Brca1-wildtype and Brca1-mutant (411) mouse embryonic fibroblasts (MEFs) transfected with Adar1 siRNA. Cells were either transfected with control, non-targeting, siRNA (siCTRL), or transfected with a titration of Adar1 siRNA SMARTpool. 72 h hours after transfection, cell lysates were generated and western blotted to detect phosphorylated CHK1 (p-CHK1), CHK1, γH2AX, cleaved PARP1 (c-PARP1), cleaved caspase 3 (c-Casp3) and caspase 3 (Casp3) proteins. All graphs show median and individual data points. Error bars represent Standard Deviation (SD). p values are as follows: ns=not significant, * p<0.05, ** p<0.005, *** p<0.0005, **** p<0.00005.

FIG. 10. A. Western blot of SUM149 BRCA1-Mut and SUM149 BRCA1-Rev cells subjected to stable RNase H1 overexpression (+RH1) and transfected with ADAR1 siRNA. Cells were transduced with a plasmid construct containing RNASEH1 cDNA to induce stable overexpression of RNase H1, and were transfected with control, non-targeting, siRNA (siCTRL), or transfected with either ADAR1 siRNA SMARTpool (P) or two individual ADAR1 siRNAs (#1, #2). 72 hours after transfection, cell lysates were generated and western blotted to detect ADAR1 and RNase H1 proteins. B. Quantification of cell survival after siRNA transfection, in conditions as described in A. After transfection, cells were continuously cultured for 6 days, after which cell viability was determined by CellTiter-Glo®. C, D. Clonogenic survival of SUM149 BRCA1-Mut and SUM149 BRCA1-Rev cells subjected to stable RNase H1 overexpression (+RH1) and transfected with ADAR1 siRNA, in conditions as described in A. After transfection, cells were continuously cultured for 10 days, after which colonies were stained and counted. All graphs show median and individual data points. Error bars represent Standard Deviation (SD) or the min to max range in case of box-and-whiskers plots. p values are as follows: ns=not significant, * p<0.05, ** p<0.005, *** p<0.0005, **** p<0.00005.

FIG. 11. A, B. Clonogenic survival of SUM149 BRCA1-Mut and SUM149 BRCA1-Rev cells co-transfected with ADAR1 siRNA and one of a series of siRNAs targeting RNA sensors. Cells were transfected with control, non-targeting, siRNA (siCTRL), or transfected with ADAR1 siRNA SMARTpool, in combination with RIG-1, MDA5, LGP2, PKR, cGAS or IFNAR1 siRNA SMARTpools. After transfection, cells were continuously cultured for 10 days, after which colonies were stained and counted. C. Quantification of cell survival in SUM149 BRCA1-Mut and SUM149 BRCA1-Rev cells subjected to stable RNase H1 overexpression (+RH1), transfected with ADAR1 siRNA and exposed to pharmacological inhibition of the JAK/STAT pathway. Cells were transduced with a plasmid construct containing RNASEH1 cDNA to induce stable overexpression of RNase H1, and were either transfected with control, non-targeting, siRNA (siCTRL) or transfected with a titration of ADAR1 siRNA SMARTpool, and continuously exposed to the JAK1/2 inhibitor ruxolitinib for 6 days, after which cell viability was determined by CellTiter-Glo®. All graphs show median and individual data points. Error bars represent Standard Deviation (SD) or the min to max range in case of box-and-whiskers plots. p values are as follows: ns=not significant, * p<0.05, ** p<0.005, *** p<0.0005, **** p<0.00005.

FIG. 12. A. Western blot of SUM149 BRCA1-Mut and SUM149 BRCA1-Rev cells transfected with ADAR1p150-selective siRNA. Cells were transfected with control, non-targeting, siRNA (siCTRL), or transfected with either ADAR1 siRNA (siADAR1) or an individual ADAR1 siRNA targeting selectively the ADAR1p150 isoform (siADAR1p150). 72 hours after transfection, cell lysates were generated and western blotted to detect ADAR1 protein. B. Quantification of cell survival after siRNA transfection. SUM149 BRCA1-Mut and SUM149 BRCA1-Rev cells were transfected with ADAR1p150-selective siRNA as described in A. After transfection, cells were continuously cultured for 6 days, after which cell viability was determined by CellTiter-Glo®. C. Western blot of HEK293T ADAR1-wildtype, ADAR1-knockout and ADAR1p150-knockout cells transfected with BRCA1 siRNA. Cells were either transfected with control, non-targeting, siRNA (siCTRL) or transfected with a titration of BRCA1 siRNA SMARTpool. 72 hours after transfection, cell lysates were generated and western blotted to detect ADAR1 and BRCA1 proteins. D. Quantification of cell survival after siRNA transfection. HEK293T ADAR1-wildtype, ADAR1-knockout and ADAR1p150-knockout cells were transfected with BRCA1 siRNA as described in C. After transfection, cells were continuously cultured for 6 days, after which cell viability was determined by CellTiter-Glo®. E. Western blot of HEK293T ADAR1-wildtype, ADAR1-knockout and ADAR1p150-knockout cells transfected with BRCA2 siRNA. Cells were either transfected with control, non-targeting, siRNA (siCTRL) or transfected with a titration of BRCA2 siRNA SMARTpool. 72 hours after transfection, cell lysates were generated and western blotted to detect ADAR1 and BRCA2 proteins. F. Quantification of cell survival after siRNA transfection. HEK293T ADAR1-wildtype, ADAR1 -knockout and ADAR1p150-knockout cells were transfected with BRCA2 siRNA as described in E. After transfection, cells were continuously cultured for 6 days, after which cell viability was determined by CellTiter-Glo®. All graphs show median and individual data points. Error bars represent Standard Deviation (SD) or the min to max range in case of box-and-whiskers plots. p values are as follows: ns=not significant, * p<0.05, ** p<0.005, *** p<0.0005, **** p<0.00005.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognise. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Amino acids are referred to herein using the name of the amino acid, the three-letter abbreviation or the single letter abbreviation.

Unless otherwise indicated, any nucleic acid sequences are written left to right in 5′ to 3′ orientation; amino acid sequences are written left to right in amino to carboxy orientation, respectively.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

Minor variations in the amino acid sequences of the proteins encoded by genes associated with homologous recombination deficiency are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins encoded by genes associated with homologous recombination deficiency according to the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN: 1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide. For example, a fragment may comprise at least 5, at least 10, at least 11, at least 12, at least 13, at least 14, at least 15, at least 16, at least 17, at least 18, at least 19, at least 20 or more amino acids of the protein from which it is derived. A fragment may be continuous or discontinuous, preferably continuous.

The nucleic acid molecules of the present invention may be prepared by any means known in the art. For example, large amounts of the nucleic acid molecules may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The nucleic acid molecules of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/ Asp | | RAY |
| Glu/ Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

A “fragment” of a nucleic acid molecule comprises a series of consecutive nucleotides from the sequence of said full-length nucleic acid molecule. By way of example, a “fragment” of a nucleic acid molecule may comprise (or consist of) at least 600 consecutive nucleotides from the sequence of said nucleic acid molecule (e.g. at least 50, 60, 70, 80, 85, 90, 95, 100 or more consecutive nucleic acid residues of said nucleic acid molecule). Typically, a fragment as defined herein retains the same function as the full-length nucleic acid molecule.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, at least about 95%, or at least about 98%, or at least about 99%, or at least about 100%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

References herein to the level of a particular molecule (e.g. ADAR1 or any of the ADAR1 inhibitors described herein) encompass the actual amount of the molecule, such as the mass, molar amount, concentration or molarity of the molecule. Preferably in the context of the invention, references to the level of a particular molecule refer to the concentration of the molecule.

The level of a molecule may be determined in any appropriate physiological compartment. Preferred physiological compartments include a tissue sample, e.g. tumour biopsy, plasma, whole blood and/or serum. The level of a molecule may be determined from any appropriate sample from an individual, e.g. a plasma sample, a blood sample and/or a serum sample. Other non-limiting examples of samples which may be tested are tissue or fluid samples urine and biopsy samples. Formalin-Fixed Paraffin-Embedded (FFPE) or formalin-fixed (FF) tissue samples are particularly preferred. Thus, by way of non-limiting example, the invention may reference the level (e.g. concentration) of a molecule (e.g. ADAR1) in a tissue sample an individual. The level of a molecule pre-treatment with an agent of the invention may be interchangeably referred to as the “baseline”.

The level of a molecule may be compared with any appropriate control. For example, a control may be obtained from a healthy individual. Alternatively, the control may be obtained from the same individual prior to treatment, or from a different individual with a tumour in the same tissue type as to be treated, but wherein the different individual has not been treated with the ADAR1 inhibitor.

The level of a molecule after treatment with an ADAR1 inhibitor of the invention may be compared with the level of the molecule in the individual pre-treatment with the ADAR1 inhibitor. Thus, the invention may be concerned with the relative level of the molecule pre- and post-treatment. The level of a molecule pre-treatment may be used to identify an individual as suitable for treatment according to the invention. Other parameters may also be used, either alone or in combination with the level of a molecule as described above, to identify an individual as suitable for treatment according to the invention. Suitable parameters to identify an individual as suitable for treatment according to the invention are known to the skilled person.

The level of a molecule may be measured directly or indirectly, and may be determined using any appropriate technique. Suitable standard techniques are known in the art, for example Western blotting and enzyme-linked immunosorbent assays (ELISAs).

As used herein, the term “R-loop” refers to a three-stranded nucleic acid structure, consisting of two antiparallel DNA strands plus one RNA strand. The RNA strand is base-paired to the template DNA strand to form a DNA: RNA hybrid, which is associated with the corresponding non-template single-stranded DNA.

As used herein, the term “cell-free DNA (cfDNA) refers to fragments of DNA that are present outside of cells that can be detected within bodily fluids, such as the blood. In blood plasma, cfDNA typically consists of double-stranded DNA fragments of around 140-170 base pairs (bp) in length that mostly originate from leukocytes.

As used herein, the term “circulating tumour DNA” (ctDNA), refers to the portion of cfDNA derived from cancer cells, which typically comprises strands of <145 bp in length and is responsible for the substantially higher plasma cfDNA concentrations often seen in patients with cancer.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In preferred embodiments, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “treat” or “treating” as used herein encompasses prophylactic treatment (e.g. to prevent onset or recurrence of cancer) as well as corrective treatment (treatment of an individual already/currently suffering from cancer). Preferably, the term “treat” or “treating” as used herein means corrective treatment. The term “treat” or “treating” encompasses treating both cancer, symptoms thereof and diseases/disorder associated therewith.

A “therapeutically effective amount” is any amount of an ADAR1 inhibitor of the invention which, when administered alone or in combination to a patient for treating HRD cancer or a symptom thereof or a disease associated therewith is sufficient to provide such treatment of the HRD cancer, or symptom thereof, or associated disease. A “prophylactically effective amount” is any amount of an ADAR1 inhibitor of the invention that, when administered alone or in combination to an individual inhibits or delays the onset or reoccurrence of HRD cancer, or a symptom thereof or disease associated therewith. In some embodiments, the prophylactically effective amount prevents the onset or reoccurrence of HRD cancer entirely. “Inhibiting” the onset means either lessening the likelihood of HRD cancer onset (or symptom thereof or disease associated therewith) or preventing the onset entirely.

The term “pharmaceutically acceptable” as used herein means approved by a regulatory agency of the Federal or a state government, or listed in the U.S. Pharmacopeia, European Pharmacopeia or other generally recognized pharmacopeia.

An “analogue” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to an individual where the substance is converted in vivo by the action of biochemicals within the patient's body, such as enzymes, to the active pharmaceutical ingredient. Examples of prodrugs include esters of carboxylic acid groups, which can be hydrolysed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH4+ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionisable groups, one forming an anion and the other a cation, which serve to balance each other. For example, amino acids such as glycine can exist in a zwitterionic form. A “zwitterion” is a salt within the meaning herein. The ADAR1 inhibitors of the present invention may take the form of salts. The term “salts” embraces addition salts of free acids or free bases which are ADAR1 inhibitors of the invention. Salts can be “pharmaceutically-acceptable salts.” The term “pharmaceutically-acceptable salt” refers to salts which possess toxicity profiles within a range that affords utility in pharmaceutical applications.

Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of ADAR1 inhibitors of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Adenosine Deaminase Acting on RNA 1 (ADAR1)

Adenosine Deaminase Acting on RNA 1 (ADAR1) catalyses the hydrolytic deamination of adenosine to inosine in double-stranded RNA (dsRNA), a process referred to as A-to-I RNA editing. A-to-I RNA editing has two major functions: first, marking endogenous RNAs as “self”, therefore helping the innate immune system to distinguish repeat- and endogenous retrovirus-derived RNAs from invading pathogenic RNAs; and second, recoding the information of the coding RNAs, leading to the translation of proteins that differ from their genomically-encoded versions.

One common feature of human cancers is their ability to evolve and adapt in the face of selective pressures such as nutrient and oxygen deprivation as well as the selective pressure of treatment. In part, cancers can evolve and adapt because of the diversity of cancer proteomes, compared to those in normal cells. In this regard, ADAR1 has previously been considered as a therapeutic target in cancer because the RNA editing that ADAR1 normally carries out acts as a barrier to extreme levels of proteomic diversity that cancers require to survive.

Another feature of human cancers is their ability to evade immune surveillance and escape elimination by the immune system. In part, cancers can develop because cancer cells elaborate strategies to compromise the ability of the immune system to recognize them. One such strategy is mediated by chronic interferon signalling and prolonged expression of interferon-stimulated genes (ISG), which enables cancer cells to escape immune elimination via upregulation of multiple immunosuppressive factors (Benci et al. (2016) Cell 167(6):154901554.e12). In this regard, ADAR1 has previously been considered as a therapeutic target in cancer because chronic tumour-intrinsic interferon signalling induces a cancer cell state that is sensitized to respond to aberrant dsRNA accumulation, thereby exposing a vulnerability of ISG signature-positive cancer cells to ADAR inhibition. Loss of ADAR1 in this context leads to activation of the translational repressors PKR and eIF2α in cancer cells, causing an overall shutdown in translation that underlies cell lethality.

Importantly, this prior art regarding ADAR1 in cancer therapy does not relate, in any aspect, with BRCA1 or BRCA2 gene dependency or HRD. The reasons that have led to consider ADAR1 as a therapeutic target in either of the above examples proceed from theories that are distinct from the mechanism underlying the present invention. Further, the synthetic lethality underpinning the present invention allow for the treatment of patient cohorts not considered in the art, such as those patients who develop resistance to PARP inhibitors, or who experience dose-limiting toxicity to such agents.

An exemplary ADAR1 is human ADAR1, a reference sequence for which is found as NCBI Accession No: NG_011844.2 (RefSeqGene, Gene ID: 103, last updated 5 Feb. 2023, accessed 21 Feb. 2023). This exemplary human ADAR1 gene gives rise to multiple transcript variants and ADAR1 protein isoforms:

| ADAR1 mRNA | | | ADAR1 protein | | |
|---|---|---|---|---|---|
| | | SEQ | | | SEQ |
| | Transcript | ID | | Iso- | ID |
| Accession No. | variant | NO | Accession No. | form | NO |
| NM_001025107.3 | 4 | 1 | NP_001020278.1 | d | 2 |
| NM_001365046.1 | 7 | 3 | NP_001351975.1 | d | 2 |
| NM_001193495.2 | 5 | 4 | NP_001180424.1 | d | 2 |
| NM_001365047.1 | 8 | 5 | NP_001351976.1 | d | 2 |
| NM_001365048.1 | 9 | 6 | NP_001351977.1 | d | 2 |
| NM_001111.5 | 1 | 7 | NP_001102.3 | a | 8 |
| NM_015840.4 | 2 | 9 | NP_056655.3 | b | 10 |
| NM_015841.4 | 3 | 11 | NP_056656.3 | c | 12 |
| NM_001365045.1 | 6 | 13 | NP_001351974.1 | e | 14 |
| NM_001365049.1 | 10 | 15 | NP_001351978.1 | f | 16 |

(wherein the version number is after the “.” by convention, all accessed 21 Feb. 2023)

Any and all references to ADAR1 herein refer to any ADAR1 gene, ADAR1 mRNA and/or ADAR1 protein sequence, particularly human ADAR1, including the exemplary sequences described herein and variants and fragments thereof.

### ADAR1 Inhibitors

The present inventors have now demonstrated that ADAR1 inhibitors elicit synthetic lethality in HRD cancer cells. Without being bound by theory, it is believed that HRD cells, such as cells with a BRCA1/2 deficiency or loss of function have difficulty repairing DNA due to the persistence of R-loops, DNA/RNA hybrid structures that form in genomic DNA which, which when encountered by replication forks, cause replication fork stalling and/or collapse. One function of ADAR1 is to facilitate the removal of persistent R-loops from the genome. Therefore, the inventors' current hypothesis is that ADAR1 inhibition results in R-loop persistence, and subsequent replication fork stalling/collapse. Whilst homologous recombination proficient normal cells can tolerate agents that cause replication fork stalling and collapse, HRD cancer cells cannot, with the result that HRD cancer cells die in the face of such agents. This is supported by the data herein, in which over-expression of the R-loop degrading enzyme RNAse H1 reverses the ADAR1/HR deficiency synthetic lethality. Further, the data herein suggests that this synthetic lethality is dependent on JAK/STAT signalling, as pharmacological inhibition of the JAK/STAT signalling pathway also reverses the ADAR1/HR deficiency synthetic lethality.

In the present invention, the term “ADAR1 inhibitor” refers to any agent, compound or substance that inhibits the expression levels and/or a biological activity of ADAR1. Some inhibitors are known and further examples may be found by the application of screening technologies to these targets. Examples of such screening methods are described herein.

An ADAR1 inhibitor according to the invention may directly or indirectly inhibit ADAR1 as described herein. Unless explicitly stated, references herein to inhibition of ADAR1 encompass both direct and indirect inhibition of ADAR1. Preferably an ADAR1 inhibitor of the invention directly inhibits ADAR1.

“Direct inhibition of ADAR1” as used herein means inhibition of the expression and/or activity of ADAR1 directly, i.e. without any intermediary step. By way of non-limiting example, direct inhibition of ADAR1 may elicited by competitive or non-competitive inhibitors of the ADAR1 enzyme or by inhibition of a gene encoding ADAR1.

“Indirect inhibition of ADAR1” as used herein means inhibition of the expression and/or activity of ADAR1 indirectly, i.e. through the modulation or delivery of genes/enzymes upstream of ADAR1 and/or through the generation or delivery of intermediaries which directly inhibit ADAR1. Indirect inhibition may, for example, be elicited by upregulating the expression of an enzyme which generates an endogenous direct inhibitor of ADAR1.

An ADAR1 inhibitor according to the invention may selectively inhibit ADAR1. This is typically the case for agents which directly inhibit ADAR1. For such agents which directly inhibit ADAR1, selectivity may mean that the agent binds selectively (also referred to interchangeably herein as specifically) with ADAR1. By “binds selectively”, it will be understood that said agent binds to ADAR1 (e.g. ADAR1 DNA, RNA or protein), with no significant cross-reactivity to any other molecule. Cross-reactivity may be assessed by any suitable method. By way of non-limiting example, cross-reactivity of an ADAR1 inhibitor with a molecule other than ADAR1 may be considered significant if the agent binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to ADAR1. An ADAR1 inhibitor that directly inhibits ADAR1 and that binds selectively to ADAR1 may bind to another molecule at less than 90%, 85%, 80%, 75%, 70%, 65%, 60%, 55%, 50%, 45%, 40%, 35%, 30%, 25% or 20% the strength that it binds to ADAR1. Preferably, the agent binds to the other molecule at less than 20%, less than 15%, less than 10% or less than 5%, less than 2% or less than 1% the strength that it binds to ADAR1.

An ADAR1 inhibitor of the invention may have off-target effects. An off-target effect is activity against a target other than ADAR1. Typically compounds with off-target effects are encompassed by the present invention if the activity against the non-ADAR1 target is not significant compared with the activity against ADAR1. Whether an off-target effect is significant may depend on the intended use of the compound. As a non-limiting example, a compound which may exert an off-target effect on the central nervous system would not be significant for a compound used in an ex vivo method as disclosed herein, but may be significant (depending on the magnitude of the off-target effect) for an in vivo therapeutic indication as disclosed herein. The presence and magnitude of any potential off-target effects can be readily assessed using standard methods known in the art.

An ADAR1 inhibitor typically decreases the expression and/or activity of ADAR1. In the context of the present invention, when referring to decreasing the expression and/or activity of ADAR1, the degree of decrease may be as defined above.

Expression may be quantified in terms of gene and/or protein expression, and may be compared with the expression of a control (e.g. housekeeping gene or protein). As a non-limiting example, in the context of ADAR1 expression, the actual amount of an ADAR1 gene, mRNA transcript and/or protein, such as the mass, molar amount, concentration or molarity of an ADAR1 gene, mRNA transcript and/or protein, or the number of mRNA molecules per cell in a sample obtained from an individual treated according to the invention and the control may be assessed, and compared with the corresponding value from the control. Alternatively, the expression of an ADAR1 gene and/or protein in a sample obtained from an individual treated according to the invention may be compared with that of the control without quantifying the mass, molar amount, concentration or molarity of the one or more gene and/or protein.

Typically, the control is an equivalent sample in which no inhibition of ADAR1 expression has been effected. As a non-limiting example, in the case where an individual is treated with an ADAR1 inhibitor that inhibits ADAR1 expression, a suitable control would be a different individual to which the ADAR1 inhibitor has not been administered or the same individual prior to administration of the compound. Conventional methods for the assessment of gene and/or protein expression are well known in the art and include RT-qPCR, ELISA, DNA microarray, RNA-Seq, serial analysis of gene expression (SAGE) and western blotting.

ADAR1 activity may be quantified in terms of A-to-I RNA editing, and may be compared with the activity of a control (i.e. recombinant enzyme of known concentration). ADAR1 activity may be quantified using any appropriate technique, examples of which are known in the art, such as the sequencing of DNA derived from reverse transcription (RT)-PCR reactions.

Typically, decreasing the expression and/or activity of ADAR1 refers to a decrease in ADAR1 expression and/or activity of at least about 5%, at least about 10%, preferably at least about 20%, at least about 25%, at least about 30%, at least about 40%, at least about 50%, or at least about 60%, or at least about 70%, or at least about 80%, at least about 90%, at least about 95%, at least about 96%, at least about 97%, at least about 98%, at least about 99% or more, up to complete inhibition of ADAR1 expression and/or activity.

An ADAR1 inhibitor of the invention may result in a decrease in the level of ADAR1 within the tissue to be treated. In this context, the level of ADAR1 encompasses, the actual amount of ADAR1, such as the mass, molar amount, concentration or molarity of ADAR1 (for a set sample size or in individual cells of said sample). Typically, the level of ADAR1 is determined in a sample obtained from an individual treated according to the invention and the control may be assessed quantitatively, and compared with the corresponding value from the control. Alternatively, the level of ADAR1 in a sample obtained from an individual treated according to the invention may be compared qualitatively with that of the control i.e. without quantifying the mass, molar amount, concentration or molarity of ADAR1.

A combination of ADAR1 inhibitors may be used to inhibit ADAR1. By way of non-limiting examples, a combination of ADAR1 inhibitors may comprise: a direct inhibitor of ADAR1 and an indirect inhibitor of ADAR1; at least two direct inhibitors of ADAR1; or at least two indirect inhibitors of ADAR1.

Any suitable ADAR1 inhibitor may be used according to the present invention. Non-limiting examples of suitable agents include small molecules, proteolysis-targeting chimeric molecules (PROTAC), macrocyclic molecules, molecular glues, nucleic acid molecules (nucleic acid inhibitors), antibodies and antigen-binding fragments thereof, antibody-drug conjugates, peptides and peptidomimetics, and aptamers, as described herein.

An ADAR1 inhibitor may be selected from a small molecule, a PROTAC, a macrocyclic molecule, a molecular glue, a nucleic acid molecule (nucleic acid inhibitor), an antibody or antigen-binding fragment thereof, an antibody-drug conjugate, a peptide or peptidomimetic, and an aptamer. Preferably an ADAR1 inhibitor is a small molecule.

### Small Molecule Inhibitors

Small molecules may be used to inhibit ADAR1 as described herein. As defined herein, small molecules are low molecular weight compounds, typically organic compounds. Typically, a small molecule has a maximum molecule weight of 900 Da, allowing for rapid diffusion across cell membranes. The maximum molecular weight of a small molecule may be 500 Da. Typically, a small molecule has a size in the order of 1 nm.

Standard techniques are known in the art for the production of small molecules, which can then readily be tested for the ability to inhibit ADAR1 as described herein.

Any small molecule which exerts an inhibitory effect on ADAR1 expression and/or activity may be used as an ADAR1 inhibitor according to the present invention. Such small molecule inhibitors may also bind to ADAR1.

It will be understood that when small molecule agents of the present invention contain one or more chiral centres, the compounds may exist in, and may be isolated as pure enantiomeric or diastereomeric forms or as racemic mixtures. The present invention therefore includes any possible enantiomers, diastereomers, racemates or mixtures thereof of small molecule agents of the invention.

Small molecule agents of the present invention may have rotameric forms, or may not have rotational activity. Rotameric forms include slow rotating forms and fast rotating forms. In some preferred embodiments, fast rotating forms of the small molecule agents of the present invention are preferred.

A small molecule agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. The invention encompasses any tautomeric form of a small molecule agent and is not to be limited merely to any one tautomeric form. Thus, small molecule agents according to the invention encompass tautomers (including keto-enol and amide-imidic acid forms).

Small molecule agents may be used in the form of pro-drugs which convert into active small molecule agents in the body, analogues or derivates, as well as in salt, hydrate and solvate forms, as defined in the Definitions section herein.

Examples of ADAR1 inhibitors that may be used in accordance with the present invention include 8-azaadenosine, 8-chloroadenosine, 8-azanebularine, AVA-ADR-001, ZYS-1 and rebecsinib.

In the present invention, a reference to 8-azaadenosine is a reference to 3H-1,2,3-Triazolo[4,5-d]pyrimidin-7-amine, 3-β-D-ribofuranosyl-(CAS No. 10299-44-2; IUPAC name 3-(β-D-Ribofuranosyl)-3H-[1,2,3]triazolo[4,5-d]pyrimidin-7-amine), with the structure:

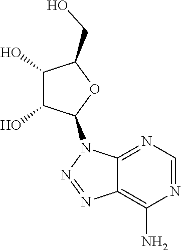 [SMILES: Nc1ncnc2c1nnn2[C@@H]1O[C@H](CO)[C@@H](O)[C@H]1O]

In the present invention, a reference to 8-chloroadenosine is a reference to 2-(6-amino-8-chloropurin-9-yl)-5-(hydroxymethyl)oxolane-3,4-diol (CAS No. 34408-14-5; IUPAC name 8-chloroadenosine), with the structure:

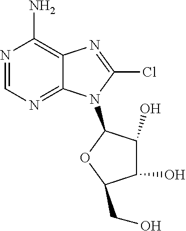 [SMILES: Nc1ncnc2c1nc(Cl)n2[C@@H]1O[C@H](CO)[C@@H](O)[C@H]1O]

In the present invention, a reference to ZYS-1 is a reference to the small molecule ADAR1 inhibitor ZYS-1 described by Wang et al. (https://doi.org/10.21203/rs.3.rs-879741/v1), which has the following structure:

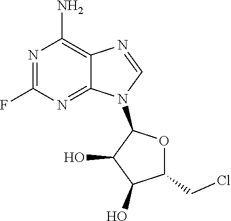 [SMILES: Nc1nc(F)nc2c1ncn2[C@H]1O[C@H](CCl)[C@@H](O)[C@H]1O]

In the present invention, a reference to 8-azanebularine is a reference to 1-[1,2,3]triazolo[4,5-d]pyrimidin-3-yl-β-D-1-deoxy-ribofuranose (CAS No. 38874-46-3), with the structure:

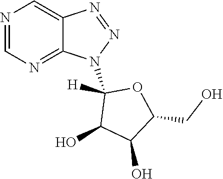 [SMILES: OC[C@H]1O[C@@H](n2nnc3cncnc32)[C@H](O)[C@@H]1O]

In the present invention, a reference to rebecsinib is a reference to 4-[4-[(5-tert-butyl-2-quinolin-6-ylpyrazol-3-yl) carbamoylamino]-3-fluorophenoxy]-N-methylpyridine-2-carboxamide (CAS No. 1020172-07-9), with the structure:

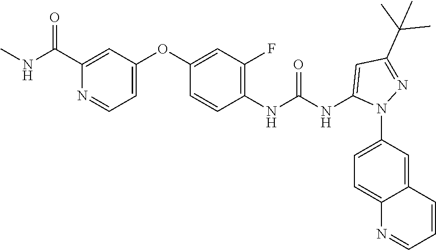 [SMILES: CNC(=O)c1cc(Oc2ccc(NC(=O)Nc3cc(C(C)(C)C)nn3-c3ccc4ncccc4c3)c(F)c2)ccn1]

### PROTACs

Proteolysis targeting chimeric molecules (also referred to as PROTACs or PROTAC reagents) may be used to inhibit ADRA1 activity as described herein. PROTACs are heterobifunctional small molecules that simultaneously bind a target protein and ubiquitin ligase, enabling ubiquitination and degradation of the target. In more detail, a PROTAC reagent typically comprises a ligand for the target protein (in the case of the present invention, ADAR1) and a ligand for an E3 ligase recognition domain. Through the use of such a PROTAC, an E3 ligase is recruited to the PROTAC-bound ADAR1, inducing ubiquitin transfer from the E3 ligase complex to the target protein (in the case of the present invention, ADAR1). Once the PROTAC has induced a sufficient degree of ubiquitination of the target, it is then recognised and degraded by the proteasome.

As a non-limiting example, a PROTAC may be produced by conjugating a ligand for an E3-ligase to a small molecule inhibitor as described herein (e.g. 8-azaadenosine or 8-chloroadenosine) or a nucleic acid such as Z-DNA (as described in Wang et al. (2024) J. Am. Chem. Soc. doi: 10.1021/jacs.3c13646, which is herein incorporated by reference) via a linker. Preferably, a PROTAC comprises a ligand for the E3 RING Cullin ligase von-Hippel Lindau protein (VHL) or cereblon—a part of a CRL4 E3 RING Cullin ligase complex, connected to a small molecule inhibitor of the invention via a linker. A PROTAC may comprise a ligand for the E3 RING Cullin ligase von-Hippel Lindau protein (VHL) connected to a small molecule inhibitor as described herein (e.g. 8-azaadenosine or 8-chloroadenosine), connected via a linker. A PROTAC may comprise cereblon (a part of a CRL4 E3 RING Cullin ligase complex) and a small molecule inhibitor as described herein (e.g. 8-azaadenosine or 8-chloroadenosine), connected via a linker.

Because of their mechanism of action, PROTACs simply need any ligand for the target protein. The functional pharmacology of the ligand, in the absence of the linker and E3 ligase ligand, is unimportant. Therefore, in some embodiments an ADAR1 inhibitory PROTAC reagent of the present invention may comprise a small molecule ADAR1 agonist as the ligand.

### SiRNA Inhibitors

Another class of ADAR1 inhibitors useful for treatment of HRD cancer includes nucleic acid inhibitors which inhibit activity or function by down-regulating production of active ADAR1 polypeptide. This can be monitored using conventional methods well known in the art, for example by screening using real time PCR.

Expression of ADAR1 may be inhibited using anti-sense or RNAi technology. The use of these approaches to down-regulate gene expression is now well-established in the art.

Thus, an ADAR1 inhibitor according to the invention may be a nucleic acid as defined herein. A nucleic acid inhibitor (nucleic acid molecule) of the invention may inhibit ADAR1 expression. Such nucleic acid inhibitors include “antisense nucleic acids”, by which is meant an RNA or DNA molecule that binds to another RNA or DNA (target RNA, DNA), whether an ADAR1 RNA or DNA as defined herein (e.g. in the case of direct ADAR1 inhibition), or a non-ADAR1 RNA or DNA (e.g. in the case of indirect inhibition). Non-limiting examples of antisense nucleic acids include, for example, antisense RNA or DNA molecules, interference RNA (RNAi), micro RNA, decoy RNA molecules, siRNA, enzymatic RNA, therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid (such as the ADAR1 gene). As such, these nucleic acids may be introduced in the form of single-stranded, double-stranded, partially single-stranded, or circular oligomeric compounds.

Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is described for example in Peyman & Ulman, Chemical Reviews, 90:543-584, 1990 and Crooke, Ann. Rev. Pharmacol. Toxicol., 32:329-376, 1992.

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example, fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g., about 15, 16 or 17 nucleotides.

An alternative to anti-sense is to use a copy of all or part of the target gene inserted in sense, that is the same orientation as the target gene, to achieve reduction in expression of the target gene by co-suppression (Angell & Baulcombe, The EMBO Journal 16(12):3675-3684, 1997 and Voinnet & Baulcombe, Nature, 389:553, 1997). Double stranded RNA (dsRNA) has been found to be even more effective in gene silencing than both sense or antisense strands alone (Fire et al, Nature 391, 806-811, 1998) dsRNA mediated silencing is gene specific and is often termed RNA interference (RNAi). Methods relating to the use of RNAi to silence genes in C. elegans, Drosophila, plants, and mammals are known in the art (Fire, Trends Genet., 15: 358-363, 19999; Sharp, RNA interference, Genes Dev. 15: 485-490 2001; Hammond et al., Nature Rev. Genet. 2: 110-1119, 2001; Tuschl, Chem. Biochem. 2: 239-245, 2001; Hamilton et al., Science 286: 950-952, 1999; Hammond, et al., Nature 404: 293-296, 2000; Zamore et al., Cell, 101: 25-33, 2000; Bernstein, Nature, 409: 363-366, 2001; Elbashir et al, Genes Dev., 15: 188-200, 2001; WO0I/29058; WO99/32619, and Elbashir et al, Nature, 411: 494-498, 2001). RNA interference is a two-step process. First, dsRNA is cleaved within the cell to yield short interfering RNAs (siRNAs) of about 21-23nt length with 5′ terminal phosphate and 3′ short overhangs (^{~}2nt). The siRNAs target the corresponding mRNA sequence specifically for destruction (Zamore, Nature Structural Biology, 8, 9, 746-750, 2001.

RNAi may also be efficiently induced using chemically synthesized siRNA duplexes of the same structure with 3′-overhang ends (Zamore et al, Cell, 101: 25-33, 2000). Synthetic siRNA duplexes have been shown to specifically suppress expression of endogenous and heterologous genes in a wide range of mammalian cell lines (Elbashir et al, Nature, 411: 494-498, 2001).

Another possibility is that nucleic acid is used which on transcription produces a ribozyme, able to cut nucleic acid at a specific site and therefore also useful in influencing gene expression, e.g., see Kashani-Sabet & Scanlon, Cancer Gene Therapy, 2(3) 213-223, 1995 and Mercola & Cohen, Cancer Gene Therapy, 2 (1) 47-59, 1995.

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs), and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending on their origin. Both types of sequence may be used to downregulate gene expression by binding to complimentary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, Plos Biology, 11 (2), 1862-1879, 2004. Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo) nucleotides, typically a UU of dTdT 3′ overhang. Based on the disclosure provided herein, the skilled person can readily design suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene downregulation or produced using expression systems (e.g. vectors). Preferably, the siRNA is synthesized synthetically.

Longer double stranded RNAs may be processed in the cell to produce siRNAs (e.g. see Myers, Nature Biotechnology, 21: 324-328, 2003). The longer dsRNA molecule may have symmetric 3′ or 5′ overhangs, e.g. of one or two (ribo) nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17:1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. Preferably, the shRNA is produced endogenously (within a cell) by transcription from a vector. shRNAs may be produced within a cell by transfecting the cell with a vector encoding the shRNA sequence under control of an RNA polymerase III promoter such as the human HI or 7SK promoter or an RNA polymerase II promoter.

Alternatively, the shRNA may be synthesised exogenously (in vitro) by transcription from a vector. The shRNA may then be introduced directly into the cell. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

The siRNA, longer dsRNA or miRNA may be produced endogenously (within a cell) by transcription from a vector. The vector may be introduced into the cell in any of the ways known in the art. Optionally, expression of the RNA sequence can be regulated using a tissue specific promoter. Alternatively, the siRNA, longer dsRNA or miRNA may be produced exogenously (in vitro) by transcription from a vector.

siRNA molecules may be synthesized using standard solid or solution phase synthesis techniques, which are known in the art. Linkages between nucleotides may be phosphodiester bonds or alternatives, e.g., linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules, which are more, or less, stable than unmodified siRNA.

The term ‘modified nucleotide base’ encompasses nucleotides with a covalently modified base and/or sugar. For example, modified nucleotides include nucleotides having sugars, which are covalently attached to low molecular weight organic groups other than a hydroxyl group at the 3′position and other than a phosphate group at the 5′position. Thus, modified nucleotides may also include 2′substituted sugars such as 2′-O-methyl-; 2-Oalkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, pseudouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 5-propyluracil, 5-propylcytosine, 5-ethyluracil, 5ethylcytosine, 5-butyluracil, 5-pentyluracil, 5-pentylcytosine, and 2,6,diaminopurine, methylpseudouracil, 1-methylguanine, 1-methylcytosine.

### Aptamers

Another class of ADAR1 inhibitors which may be used according to the present invention are aptamer.

Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, U.S. Pat. Nos. 5,654,151, 5,503,978, 5,567,588 and WO 96/38579.

The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Antibodies

Antibodies may be employed in the present invention as an example of a class of inhibitor useful for treating HRD cancer, and more particularly as inhibitors of ADAR1.

They may also be used in the methods disclosed herein for assessing an individual having cancer or predicting the response of an individual having cancer, in particular for determining whether the individual has an HRD cancer that might be treatable according to the present invention.

As used herein, the term “antibody” includes an immunoglobulin whether natural or partly or wholly synthetically produced. The term also covers any polypeptide or protein comprising an antibody binding domain. Antibody fragments which comprise an antigen binding domain are such as Fab, scFv, Fv, dAb, Fd; and diabodies. It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2,188,638 A or EP 0 239 400 A.

Antibodies can be modified in a number of ways and the term “antibody molecule” should be construed as covering any specific binding member or substance having an antibody antigen-binding domain with the required specificity. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP 0 120 694 A and EP 0 125 023 A.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′) 2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242; 423-426, 1988; Huston et al, PNAS USA, 85:5879-5883, 1988); (viii) bispecific single chain Fv dimers (WO 93/11161) and (ix) “diabodies”, multivalent or multispecific fragments constructed by gene fusion (WO 94/13804; Holliger et al, P.N.A.S. USA, 90: 6444-6448, 1993); (x) immunoadhesins (WO 98/50431). Fv, scFv or diabody molecules may be stabilised by the incorporation of disulphide bridges linking the VH and VL domains (Reiter et al, Nature Biotech, 14:1239-1245, 1996). Minibodies comprising a scFv joined to a CH3 domain may also be made (Hu et al, Cancer Res., 56:3055-3061, 1996).

Preferred antibodies used in accordance with the present invention are isolated, in the sense of being free from contaminants such as antibodies able to bind other polypeptides and/or free of serum components. Monoclonal antibodies are preferred for some purposes, though polyclonal antibodies are within the scope of the present invention.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or noncovalently. Linkage via a peptide bond may be as a result of recombinant expression of a gene fusion encoding antibody and reporter molecule. One favoured mode is by covalent linkage of each antibody with an individual fluorochrome, phosphor or laser exciting dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

An antibody of the invention and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to the human patient.

Especially preferred are human or humanized antibodies, especially as recombinant human or humanized antibodies as defined herein.

An antibody of the invention and antigen-binding fragments thereof disclosed herein can be further modified using conventional techniques known in the art, for example, by using amino acid deletion(s), insertion(s), substitution(s), addition(s), and/or recombination(s) and/or any other modification(s) known in the art, either alone or in combination. Methods for introducing such modifications in the DNA sequence underlying the amino acid sequence of an immunoglobulin chain arc well known to the person skilled in the art.

The antibodies of the invention or antigen-binding fragments thereof may have any antibody format. For example, the antibody may have a “conventional” format described above. Alternatively, the antibody may comprise or consist of a Fab fragment. The antibody according to the invention can also be a Fab′, an Fv, an scFv, an Fd, a V NAR domain, an IgNAR, an intrabody, an IgG CH2, a minibody, a single-domain antibody, an Fcab, an scFv-Fc, F(ab′)2, a di-scFv, a bi-specific T-cell engager (BiTE®), a F(ab′)3, a tetrabody, a triabody, a diabody, a DVD-Ig, an (scFv)2, or a mAb2.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid. Antibodies may modulate the activity of the polypeptide to which they bind and so, if that polypeptide has a deleterious effect in an individual, may be useful in a therapeutic context (which may include prophylaxis).

### Antibody-Drug Conjugates

An ADC comprises or consists of three components-a monoclonal antibody, a linker group and a cytotoxic drug.

An antibody of the invention may be present in an antibody-drug conjugate (ADC). In such ADCs the drug may be any which is suitable for use in the treatment of an HRD cancer, such as those described herein.

Alternatively, a non-antibody ADAR1 inhibitor of the invention may be conjugated to an antibody which can specifically bind to a target HRD cancer cell of the invention. In such ADCs, the ADAR1 inhibitor is the “drug” part of the ADC.

An ADC of the invention may comprise a cleavable or non-cleavable linker. Non-limiting examples of cleavable linkers include hydrazone, disulphide and peptide linkers. Non-limiting examples of non-cleavable linkers include those based on a maleimide-type structure such as maleimidocaproyl and 4-maleimidomethyl cyclohexane-1-carboxylate linkers.

### Peptides and Peptidomimetics

In addition, the invention encompasses the use of peptide and peptidomimetic inhibitors of ADAR1. For example, the use of peptides, stapled peptides, peptoids and peptidomimetics that would directly or indirectly inhibit ADAR1 is embraced by the present invention.

Peptidomimetics are compounds which mimic a natural peptide or protein with the ability to interact with the biological target and produce the same biological effect. Peptidomimetics may have advantages over peptides in terms of stability and bioavailability associated with a natural peptide. Peptidomimetics can have main- or side-chain modifications of the parent peptide designed for biological function. Examples of classes of peptidomimetics include, but are not limited to, peptoids and β-peptides, as well as peptides incorporating D-amino acids.

Stapled peptides comprise an external brace that forces the peptide to adopt an a-helical conformation. Starting from the native peptide sequence, two amino acids on the same face of the helix are substituted for non-native amino acids which have side chains that can be crosslinked. This crosslinking forms the external brace which holds the peptide in the desired a-helical conformation. A peptide may comprise multiple “staples”, e.g. crosslinking may occur between two, three or more pairs of modified amino acids, with each crosslinked pair forming a staple. Peptides with multiple staples are sometimes referred to as stitched peptides. Stapling peptides can increase target affinity, increase cell penetration, and protect against proteolytic degradation.

Methods for producing synthetic peptides and peptidomimetics (such as peptoids) are known in the art, as are the sequences of ADAR1 and its ligands. Techniques for stapling peptides are also described in the art, and include both covalent and non-covalent stapling strategies, with techniques that generate all-hydrocarbon covalent links being preferred. Non-limiting examples include ring-closing metathesis and olefin metathesis. Thus, it would be routine for one of skill in the art to produce suitable synthetic peptides, including stapled peptides, and peptidomimetics which directly or indirectly inhibit ADAR1 using known techniques and based on the known sequence and structures of ADAR1 and ADAR1 targets.

### Macrocyclic Molecules

In addition, the invention encompasses the use of macrocyclic molecules as inhibitors of ADAR1. For example, the use of macrocyclic molecules that would directly or indirectly inhibit ADAR1 is embraced by the present invention.

A macrocyclic molecule (also referred to as a macrocycle) is a molecule that contains a cyclic framework of at least twelve atoms. Although the size of naturally occurring macrocycles can reach 50+ atoms in the largest ring, a recent analysis of natural products suggested that 14-, 16-, and 18-membered frameworks are the most common naturally occurring macrocyclic scaffolds.

Cyclization of a linear molecule into a macrocyclic ring constitutes a significant change in molecular shape, biological activity, and drug-like properties. Compared with corresponding acyclic linear molecules, cyclised molecules typically have better physicochemical properties, such as good solubility, lipophilicity, metabolic stability, bioavailability and overall pharmacokinetics.

Methods for producing s macrocyclic molecules are known in the art, as are the sequences of ADAR1 and its ligands. Thus, it would be routine for one of skill in the art to produce suitable macrocyclic molecules which directly or indirectly inhibit ADAR1 using known techniques and based on the known sequence and structures of ADAR1 and ADAR1 targets. By way of non-limiting example, high-dilution chemistry can be used to produce macrocycles, wherein large amounts of solvent and low concentrations are added (typically slowly), to allow ring formation to occur whilst preventing nascent macrocyclic molecules from reacting with each other and polymerising. Alternatively, template synthetic techniques, typically using transition metals, to organise components of the reaction and guide them towards the desired ring formation.

### Molecular Glues

Molecular glues may be used to inhibit ADRA1 activity as described herein. Like PROTAC reagents, molecular glues are small molecules. However, whereas PROTAC reagents simultaneously bind a target protein and ubiquitin ligase, molecular glues interact with only one of the target protein (ADAR1 according to the invention) or the ubiquitin ligase. Typically molecular glues interact only with the ubiquitin ligase. This interaction stabilises the protein-protein interaction between the ubiquitin ligase and its target (ADAR1), forming ternary complexes which induce ubiquitination and degradation of the target.

Molecular glues typically have lower molecular weight, higher cell permeability and better oral absorption compared with PROTAC reagents.

A molecular glue may bind the E3 ligase Cereblon (CRBN) or an aryl sulfonamides that engages DCAF15. Examples of such molecular glues include immunomodulatory imide drugs (ImiD) such as thalidomide, lenalidomide and pomalidomide. Other molecular glues that induce protein degradation through various non-E3 ligase mechanisms of action include autophagy-mediated protein degradation, protein-protein interaction stabilisation, KRAS mutant inhibition, microtubule stabilisation stabilization, and mTOR inhibition. In Non-limiting examples of naturally occurring compounds which can function as molecular glues include rapamycin, cyclosporin A, voclosporine and sanglifehrin A. Further examples of molecular glues are described in Geiger et al. Curr. Res. Chem. Biol. 2(2022):100018, which is herein incorporated by reference in its entirety.

### Homologous Recombination Defective (HRD) Cancer

The invention relates to the treatment of homologous recombination defective (HRD) cancer. As described herein, the invention particularly relates to the treatment of HRD cancer by a mechanism of synthetic lethality. In particular, ADAR1 inhibitors elicit synthetic lethality in HRD cancer cells, which may be described as ADAR1/HR deficiency synthetic lethality, with ADAR1/BRCA (BRCA1 and/or BRCA2) synthetic lethality being a particular focus of the invention.

Homologous recombination (HR) is a process by which DNA lesions are repaired via the use of a homologous DNA sequence template, normally found on the homologous chromosome in meiotic cells or on sister chromatids in mitotic cells. The use of the homologous DNA sequence template in HR results in error-free, conservative, DNA repair. Additionally, during DNA replication, HR repair (HRR) pathways support the recovery of stalled replication forks. Successful HRR depends on several properly functioning proteins, with BRCA1 and BRCA2 proteins playing critical roles. BRCA1 is a tumor suppressor protein central to several macromolecular complexes which drive HRR and cell cycle progression. MRN and CtIP are involved in DNA resection, after which BRCA1 travels to sites of DSBs where it participates in DNA damage signaling and coordinates DNA damage repair. During the synthesis phase of normal cell cycle progression, if DNA becomes damaged, BRCA1 protein complexes recruit BRCA2 protein complexes to initiate strand invasion and/or homology-directed repair. The HR capacity of cells and cancers can be defined in several ways including the presence of deleterious BRCA1 or BRCA2 mutations; the presence of deleterious mutations in other genes that control HR; the presence of hypermethylation of the promoter region of one or more gene that controls HR, such as BRCA1, PALB2, RAD51, RAD51C, and ATR, preferably the promoter region of BRCA1; the inability to form RAD51 nuclear foci; a clinical response to platinum-based chemotherapy or PARP inhibitor; or the presence of a genomic DNA scar reflective of a HR defect in the lineage of a cancer.

The HR pathway is essential for high-fidelity DNA double strand break (DSB) repair. In HRD cancers, loss of function of one or more gene associated with HR leads HR deficiency, resulting in increased levels of genomic alterations.

As used herein, the term “homologous recombination defective cancer” (“HRD cancer”) is used to refer to any cancer which has a deficiency (complete or partial) in HR or HRR. A deficiency in HR or HRR may result from a deficiency in one or more gene involved in HRR, and/or upregulation of one or more miRNA which target a gene involved in HRR. Typically, an HRD cancer has a deficiency in in one or more gene involved in HRR. Said deficiency may be a loss-of-function mutation, or deletion of said one or more gene, with loss-of-function mutations being more common.

Non-limiting examples of miRNA which may be upregulated and result in a deficiency in HR or HRR include miR-182 and miR-1255b (which targets BRCA1), miR-148b (which targets BRCA2) and miR-193b (which targets RAD51).

Typically, HR deficiency is associated with a mutation (e.g. a loss-of-function mutation) or other deficiency such as epigenetic silencing in any gene associated with HR or HRR. Non-limiting examples of such genes include BRCA1, BRCA2, ATM, BARD1, PALB2, BRIP1, RAD51B, RAD51C, RAD51D, CDK12, FAAP20, CHEK2, FAN1, FANCE, FANCM, and POLQ. Thus, the invention relates to the treatment of cancers which have a mutation (e.g. a loss-of-function mutation) or other deficiency in one or more gene associated with HR or HRR, including but not limited to BRCA1, BRCA2, ATM, BARD1, PALB2, BRIP1, RAD51B, RAD51C, RAD51D, CDK12, FAAP20, CHEK2, FAN1, FANCE, FANCM, and POLQ. In other words, according to the invention, ADAR1 inhibitors may be used in the treatment of HRD cancers, wherein the HR deficiency may be associated with a mutation (e.g. a loss-of-function mutation) or other deficiency (e.g. epigenetic silencing) in one or more gene associated with HR or HRR, including but not limited to BRCA1, BRCA2, ATM, BARD1, PALB2, BRIP1, RAD51B, RAD51C, RAD51D, CDK12, FAAP20, CHEK2, FAN1, FANCE, FANCM, and POLQ. Preferably the invention relates to the treatment of HRD cancers which have a mutation (e.g. a loss-of-function mutation) or other deficiency (e.g. epigenetic silencing) in BRCA1 and/or BRCA2, with the treatment of HRD cancers which have a mutation (e.g. a loss-of-function mutation) or other deficiency (e.g. epigenetic silencing) in BRCA1 being particularly preferred.

The nature of the (loss-of-function) mutation or other deficiency (e.g. epigenetic silencing) in the one or more gene associated with HR or HRR, including BRCA1, BRCA2, ATM, BARD1, PALB2, BRIP1, RAD51B, RAD51C, RAD51D, CDK12, FAAP20, CHEK2, FAN1, FANCE, FANCM, and POLQ is not particularly limited according to the invention, provided that the (loss-of-function) mutation or other deficiency (e.g. epigenetic silencing) results in a deficiency in HR or HRR.

For the avoidance of doubt, any reference herein to HRD cancer applies equally and without reservation to HDR cancer cells. By way of non-limiting example, HRD cancer cells typically have a deficiency in one or more gene involved in HRR, such as those described herein.

HRD is a common characteristic of many tumors. Non-limiting examples of types of cancer known to be associated with a (loss-of-function) mutation or other deficiency in one or more gene associated with HR or HRR include ovarian cancer, breast cancer, pancreatic cancer, prostate cancer, adrenal cancer, uterine cancer, biliary cancer, cancer of the urinary tract, head and neck cancer, bone/soft tissue cancer, lymphoid cancer, liver cancers, mesothelioma, oesophageal cancer, neuroendocrine tumours, lung cancer, colorectal cancer and skin cancer. Thus, the invention relates to the treatment of HRD cancers including ovarian cancer, breast cancer, pancreatic cancer, prostate cancer, adrenal cancer, uterine cancer, biliary cancer, cancer of the urinary tract, head and neck cancer, bone/soft tissue cancer, lymphoid cancer, liver cancers, mesothelioma, oesophageal cancer, neuroendocrine tumours, lung cancer, colorectal cancer and skin cancer. Ovarian cancer, breast cancer, pancreatic cancer, and prostate cancer are particularly associated with (loss-of-function) mutations or other deficiencies in one or more gene associated with HR or HRR. Therefore, the treatment of HRD ovarian cancer, breast cancer, pancreatic cancer, biliary tract cancer and prostate cancer is preferred according to the invention. The invention also relates to the treatment of HRD ovarian cancer, pancreatic cancer, biliary tract cancer and prostate cancer.

In some preferred embodiments, the invention relates to the treatment of HRD cancer, particularly ovarian cancer, breast cancer, pancreatic cancer, biliary tract cancer or prostate cancer, that is associated with a (loss-of-function) mutation in BRCA1 and/or BRCA2.

The invention may relate to the treatment of cancers that are sensitive to treatment with one or more PARP inhibitor. Alternatively, the invention may relate to the treatment of cancers that are insensitive to treatment with one or more PARP inhibitor. When the invention relates to the treatment of cancers that are insensitive to treatment with one or more PARP inhibitor, said cancers may have previously been sensitive to treatment with one or more PARP inhibitor, but have developed resistance to one or more PARP inhibitor following treatment therewith.

The invention may relate to the treatment of breast cancer that (i) has a (loss-of-function) mutation in BRCA1 and/or BRCA2; (ii) has a (loss-of-function) mutation in BRCA1 and/or BRCA2 and is human epidermal growth factor 2 negative (HER2⁻); or (iii) is triple-negative (Estrogen Receptor negative (ER⁻), Progesterone Receptor negative (PR⁻) and HER2⁻).

### Treatment of HRD Cancer

The present invention provides methods and medical uses for the treatment of HRD cancers with ADAR1 inhibitors.

In particular, the invention provides ADAR1 inhibitors for use in a method of treating HRD cancers. The invention also provides a method of treating HRD cancer, said method comprising administering a therapeutically effective amount of an ADAR1 inhibitor to an individual in need thereof. The invention also relates to the use of an ADAR1 inhibitor in the manufacture of a medicament for treating an HRD cancer. These medical uses and methods exploit the ADAR1/HR deficiency synthetic lethality identified by the present inventors, particularly ADAR1/BRCA (BRCA1 and/or BRCA2, particularly BRCA1) synthetic lethality.

All disclosure herein in relation to ADAR1 inhibitors for the treatment of HRD cancer apply equally and without reservation to all medical uses and methods of treatments described herein unless expressly stated to the contrary.

A method of treating a HRD cancer according to the invention may comprise determining whether the cancer to be treated is an HRD cancer. If the cancer is determined to be an HRD cancer, then a therapeutically effective amount of an ADAR1 inhibitor may be administered to the individual.

Any appropriate means or method may be used to determine whether the cancer is an HRD cancer. By way of non-limiting example, an HRD cancer may be identified as such by testing a sample comprising cancer cells from an individual, for example to determine whether one or more cancer cells in said sample comprise a (loss-of-function) mutation or other deficiency in one or more gene associated with a deficiency in HR or HRR, such as those genes identified herein.

Non-limiting examples of types of cancer known to be associated with a (loss-of-function) mutation or other deficiency in one or more gene associated with HR or HRR are set out above. Thus, non-limiting examples of HRD cancer include ovarian cancer, breast cancer, pancreatic cancer, prostate cancer, adrenal cancer, uterine cancer, biliary cancer, cancer of the urinary tract, head and neck cancer, bone/soft tissue cancer, lymphoid cancer, liver cancers, mesothelioma, oesophageal cancer, neuroendocrine tumours, lung cancer, colorectal cancer and skin cancer. Thus, the invention relates to the treatment of HRD cancers including ovarian cancer, breast cancer, pancreatic cancer, prostate cancer, adrenal cancer, uterine cancer, biliary cancer, cancer of the urinary tract, head and neck cancer, bone/soft tissue cancer, lymphoid cancer, liver cancers, mesothelioma, oesophageal cancer, neuroendocrine tumours, lung cancer, colorectal cancer and skin cancer. Ovarian cancer, breast cancer, pancreatic cancer, prostate cancer are particularly associated with (loss-of-function) mutations or other deficiencies in one or more gene associated with HR or HRR. Therefore, the treatment of HRD ovarian cancer, breast cancer, pancreatic cancer, biliary tract cancer and prostate cancer is preferred according to the invention. The invention also relates to the treatment of HRD ovarian cancer, pancreatic cancer, biliary tract cancer and prostate cancer.

The HRD cancer may be characterised by one or more mutation (e.g. a loss-of-function mutation) or other deficiency (e.g. epigenetic silencing) in one or more gene associated with HR or HRR, including but not limited to BRCA1, BRCA2, ATM, BARD1, PALB2, BRIP1, RAD51B, RAD51C, RAD51D, CDK12, FAAP20, CHEK2, FAN1, FANCE, FANCM, and POLQ. Preferably the HRD cancer may be characterised by one or more mutation (e.g. a loss-of-function mutation) or other deficiency (e.g. epigenetic silencing) in BRCA1 and/or BRCA2, BRCA1 being particularly preferred.

Said one or more mutation (e.g. a loss-of-function mutation) or other deficiency (e.g. epigenetic silencing) in one or more gene associated with HR or HRR (e.g. BRCA1 and/or BRCA2) may occur in somatic pre-cancerous or cancerous cells. While such mutations (e.g. a loss-of-function mutation) or other deficiency (e.g. epigenetic silencing) in one or more gene associated with HR or HRR (e.g. BRCA1 and/or BRCA2) are mostly believed to be somatic, any of the one or more mutation (e.g. a loss-of-function mutation) or other deficiency (e.g. epigenetic silencing) in one or more gene associated with HR or HRR (e.g. BRCA1 and/or BRCA2) mutations may be associated with clonal haematopoiesis, e.g. as a result of ageing, and there may also be HRD cancers characterised by one or more mutation (e.g. a loss-of-function mutation) or other deficiency (e.g. epigenetic silencing) in one or more gene associated with HR or HRR (e.g. BRCA1 and/or BRCA2) occurring in the germ line of the individual patient.

Thus, an ADAR1 inhibitor may be used in a method of treating an HRD cancer according to the invention wherein said method comprises determining whether the cancer is HRD by determining the presence of a deficiency and/or mutation in one or more gene associated with homologous recombination deficiency, optionally a deficiency and/or mutation in one or more gene selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ, preferably BRCA1 and/or BRCA2.

An HRD cancer may be identified as such by testing a sample comprising cancer cells from an individual to determine the expression of one or more gene associated with HR or HRR, such as those described herein (e.g. BRCA1 and/or BRCA2) to evaluate whether expression of the protein is absent or at a reduced level compared to normal.

Alternatively or in addition, an HRD cancer may be characterised by the cancer cells having a defect in or the cancer cells exhibiting epigenetic inactivation of one or more gene associated with HR or HRR, such as those described herein (e.g. BRCA1 and/or BRCA2), or loss of protein function.

More generally, a cancer may be identified as an HRD cancer by determining the activity of the proteins encoded by one or more gene associated with HR or HRR, such as those described herein (e.g. BRCA1 and/or BRCA2) in a sample of cells from an individual. The sample may be of normal cells from the individual where the individual has a mutation in one or more gene associated with HR or HRR, such as those described herein (e.g. BRCA1 and/or BRCA2) or the sample may be of cancer cells, e.g. where the cells forming a tumour exhibit defects in the activity of the protein encoded by one or more gene associated with HR or HRR, such as those described herein (e.g. BRCA1 and/or BRCA2). Activity may be determined relative to a control, for example in the case of defects in cancer cells, a relative to non-cancerous cells, preferably from the same tissue. The activity of the one or more gene associated with HR or HRR, such as those described herein (e.g. BRCA1 and/or BRCA2) may be determined by using techniques well known in the art such as Western blot analysis, immunoprecipitation, immunohistology, chromosomal abnormalities, enzymatic or DNA binding assays, and plasmid-based assays.

The sample may comprise or consist of normal cells from the individual where the individual has a mutation in one or more gene associated with HR or HRR, such as those described herein (e.g. BRCA1 and/or BRCA2) or the sample may comprise or consist of cancer cells, e.g. where the cells forming a tumour contain one or more mutation in one or more gene associated with HR or HRR, such as those described herein (e.g. BRCA1 and/or BRCA2). Activity may be determined relative to a control, for example in the case of defects in cancer cells, relative to non-cancerous cells, preferably from the same tissue.

The determination of expression of one or more gene associated with HR or HRR, such as those described herein (e.g. BRCA1 and/or BRCA2) may involve determining the presence or amount of said one or more gene mRNA in a sample. Methods for doing this are well known to the skilled person. By way of example, they include determining the presence of mRNA of one or more gene associated with HR or HRR, such as those described herein (e.g. BRCA1 and/or BRCA2) (i) using a labelled probe that is capable of hybridising to the nucleic acid of said one or more gene; and/or (ii) using PCR involving one or more primers based on a nucleic acid sequence of said one or more gene to determine whether the transcript of said one or more gene is present in a sample. The probe may also be immobilised as a sequence included in a microarray. It is also possible to use quantitative PCR or nanostring nCounter technology to assess the downstream consequences of mutation.

Detecting mRNA of one or more gene associated with HR or HRR, such as those described herein (e.g. BRCA1 and/or BRCA2) may be carried out by extracting RNA from a sample of the tumour and measuring expression of said one or more gene specifically using quantitative real time RT-PCR. Alternatively or additionally, the expression of said one or more gene could be assessed using RNA extracted from a tumour sample using microarray analysis, which measures the levels of mRNA for a group of genes using a plurality of probes immobilised on a substrate to form the array.

A cancer may be identified as an HRD cancer by determining the presence in a cell sample from an individual's tumour of one or more chromosomal abnormalities, for example deletions in part or loss of entire chromosomes, corresponding to gene loss. Chromosomal abnormalities may be visualised through any karyotyping technique known in the art, including but not limited to Giemesa staining, quinacrine staining, Hoechst 33258 staining, DAPI (4′-6-diamidino-2-phenylindole) staining, daunomycin staining, and fluorescence in situ hybridization.

A cancer may be identified as an HRD cancer by determining the presence in a cell sample from the individual of one or more variations, for example, polymorphisms or mutations, in the nucleic acid sequence of one or more gene associated with HR or HRR, such as those described herein (e.g. BRCA1 and/or BRCA2).

Alternatively or in addition, an HRD cancer may be identified by determining the presence in the circulating tumour DNA (ctDNA) and/or cell-free DNA (cfDNA) in a patient. ctDNA and/or cfDNA may be extracted from a blood sample from the patient. The presence of one or more variations, for example, polymorphisms or mutations, in the nucleic acid sequence of one or more gene associated with HR or HRR, such as those described herein (e.g. BRCA1 and/or BRCA2) in ctDNA and/or cfDNA from the patient may be used to identify a cancer as an HRD cancer. Thus, analysis of ctDNA and/or cfDNA of a patient for variations, for example, polymorphisms or mutations, in the nucleic acid sequence of one or more gene associated with HR or HRR, such as those described herein (e.g. BRCA1 and/or BRCA2) may be used to identify a cancer as an HRD cancer.

Sequence variations such as mutations and polymorphisms may include a deletion, insertion or substitution of one or more nucleotides, relative to the wild-type nucleotide sequence. The one or more variations may be in a coding or non-coding region of the nucleic acid sequence and may reduce or abolish the expression or function of the one or more gene associated with HR or HRR, such as those described herein (e.g. BRCA1 and/or BRCA2). In other words, the variant nucleic acid may encode a variant polypeptide which has reduced or abolished activity or may encode a wild-type polypeptide which has little or no expression within the cell, for example through the altered activity of a regulatory element. A variant nucleic acid may have one or more mutations or polymorphisms relative to the wild-type sequence.

Alternatively or additionally, in the present invention the determination of whether a patient has an HRD cancer can be carried out by analysis of expression of the protein encoded by one or more gene associated with HR or HRR, such as those described herein (e.g. BRCA1 and/or BRCA2), for example by examining whether levels of said protein (e.g. BRCA1 and/or BRCA2) are suppressed.

In some aspects, the presence or amount of protein encoded by one or more gene associated with HR or HRR, such as those described herein (e.g. BRCA1 and/or BRCA2) may be determined using a binding agent capable of specifically binding to said protein, or fragments thereof. A preferred type of protein binding agent is an antibody capable of specifically binding to said protein or fragment thereof (e.g. BRCA1 and/or BRCA2). The antibody may be labelled to enable it to be detected or capable of detection following reaction with one or more further species, for example using a secondary antibody that is labelled or capable of producing a detectable result, e.g. in an ELISA type assay. As an alternative, a labelled binding agent may be employed in a western blot to detect said protein (e.g. BRCA1 and/or BRCA2).

Alternatively, or additionally, the method for determining the presence of a protein encoded by one or more gene associated with HR or HRR, such as those described herein (e.g. BRCA1 and/or BRCA2) may be carried out on tumour samples, for example using immunohistochemical (IHC) analysis or in situ RNA-hybridisation. IHC analysis can be carried out using paraffin fixed samples or frozen tissue samples, and generally involves staining the samples to highlight the presence and location of said protein (e.g. BRCA1 and/or BRCA2).

As described herein, an ADAR1 inhibitor may be used in a method of treating an HRD cancer according to the invention wherein sad method comprises determining whether the cancer is HRD by determining the presence of a deficiency and/or mutation in one or more gene associated with homologous recombination deficiency, optionally a deficiency and/or mutation in one or more gene selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ, preferably BRCA1 and/or BRCA2.

The step of determining the presence of a deficiency and/or mutation in said one or more gene associated with homologous recombination deficiency (e.g. BRCA1 and/or BRCA2) may be performed on nucleic acid sequences obtained from an individual's cancerous and/or noncancerous cells, using any standard technique known in the art, examples of which are described herein. Non-limiting examples of suitable techniques for determining the presence of a deficiency and/or mutation in one or more gene associated with homologous recombination deficiency (e.g. BRCA1 and/or BRCA2) using nucleic acid sequences obtained from an individual's cancerous and/or noncancerous cells include direct sequencing, hybridisation to a probe, restriction fragment length polymorphism (RFLP) analysis, single-stranded conformation polymorphism (SSCP), PCR amplification of specific alleles, amplification of DNA target by PCR followed by a mini-sequencing assay, allelic discrimination during PCR, Genetic Bit Analysis, pyrosequencing, oligonucleotide ligation assay, analysis of melting curves, testing for a loss of heterozygosity (LOH) or next generation sequencing (NGS) techniques, single molecule sequencing techniques or nanostring nCounter technology.

Alternatively or additionally, the step of determining the presence of a deficiency and/or mutation in said one or more gene associated with homologous recombination deficiency (e.g. BRCA1 and/or BRCA2) may comprise measuring/quantifying protein expression of the one or more gene associated with homologous recombination deficiency (e.g. BRCA1 and/or BRCA2) in a sample obtained from the individual (which may comprise or consist of cancerous and/or non-cancerous cells). Any standard technique known in the art, may be used to measure/quantify the protein expression of the one or more gene associated with homologous recombination deficiency, examples of which are described herein. Non-limiting examples of suitable techniques for measuring/quantifying protein expression of the one or more gene associated with homologous recombination deficiency in a sample obtained from the individual (which may comprise or consist of cancerous and/or non-cancerous cells) include immunohistochemistry, determining protein levels in a cell lysate by ELISA or Western blotting, and/or determining protein expression using a binding agent capable of specifically binding to a protein, or a fragment thereof.

Alternatively or additionally, the step of determining the presence of a deficiency and/or mutation in said one or more gene associated with homologous recombination deficiency (e.g. BRCA1 and/or BRCA2) may comprise extracting RNA from a sample of an individual's cancerous and/or noncancerous cells and measuring/quantifying the RNA of the one or more gene associated with homologous recombination deficiency (e.g. BRCA1 and/or BRCA2). Any standard technique known in the art may be used, examples of which are described herein. Non-limiting examples of suitable techniques for measuring/quantifying the RNA of the one or more gene associated with homologous recombination deficiency (e.g. BRCA1 and/or BRCA2) include real time PCR and/or by using a probe capable of hybridising to the RNA of one or more gene associated with homologous recombination deficiency RNA. Said probe may be immobilised in a microarray.

Alternatively or additionally, the step of determining the presence of a deficiency and/or mutation in said one or more gene associated with homologous recombination deficiency (e.g. BRCA1 and/or BRCA2) may comprise identifying gene loss resulting from chromosomal instability through karyotype analysis of a sample obtained from the individual.

Alternatively or additionally, the step of determining the presence of a deficiency and/or mutation in said one or more gene associated with homologous recombination deficiency (e.g. BRCA1 and/or BRCA2) may comprise identifying specific signatures (“genomic scars”) through whole genome sequencing (e.g. HRDetect) or shallow whole genome sequencing.

As described herein, an ADAR1 inhibitor may be used in a method of treating an HRD cancer according to the invention wherein sad method comprises determining whether the cancer is an HRD cancer. Additional techniques may be used to determine whether a cancer is an HRD cancer, in combination with or as an alternative to determining the presence of a deficiency and/or mutation in one or more gene associated with homologous recombination deficiency, as described herein.

Thus, an ADAR1 inhibitor may be used in a method of treating an HRD cancer according to the invention wherein sad method comprises determining whether the cancer is an HRD cancer by (a) use of a companion diagnostic for homologous recombination deficiency; (b) detecting and/or quantifying RAD51 foci within a sample of cancerous and/or non-cancerous cells from an individual, wherein reduced RAD51 signal is associated with homologous recombination deficiency; (c) detecting and/or quantifying a homologous recombination deficiency defect, optionally a transcriptomic signature and/or mutational scar associated with homologous recombination deficiency such as, but not exclusive to, the Myriad MyChoice assay; and/or (d) detecting and/or quantifying platinum sensitivity of the cancer, wherein platinum sensitivity is associated with homologous recombination deficiency.

A companion diagnostic is a diagnostic test, which provides information that facilitates the safe and effective use of a corresponding drug, in this case the use of an ADAR1 inhibitor according to the invention. A number of companion diagnostics tests are available for HRD cancers. For example, companion diagnostics for homologous recombination deficiency may determine and/or quantify loss of heterozygosity (LOH), typically genome-wide LOH, telomeric allelic imbalance (LAI) and/or large-scale state transitions (LST), or any combination thereof.

The term “loss of heterozygosity” or “LOH” refers to a common form of allelic imbalance by which a heterozygous somatic cell becomes homozygous because one of the two alleles of a gene gets lost. There are two types of LOH: (i) copy number neutral LOH which refers to a change in the gene without a change in the chromosomal copy number; and (ii) deletion LOH, which occurs as a result of copy number loss.

The term “telomeric allelic imbalance” or “LAI” refers to a chromosomal aberration where the telomeric and subtelomeric regions of a chromosome does not have the expected 1:1 ratio for the alleles and telomeric sequences inherited from the two parental chromosomes. TAI is similar to LOH, but the difference is that the structural change occurs specifically at the telomere.

The term “large-scale state transitions” or “LST” refers to chromosomal breaks that generate fragments of 10 Mb or larger. These LST can be caused by transfer of DNA from one chromosome to another chromosome, but can also be caused by inversions, deletions and duplications of DNA. Quantification of LSTs can be used as a surrogate measure for genomic instability.

Any suitable companion diagnostic may be used to determine whether a cancer is an HRD cancer according to the present invention. Suitable companion diagnostics, including those which determine one or more of LOH, LAI and/or LST are known in the art. Companion diagnostics for HRD cancer are also commercially available, and include MyChoice® CDx (from Myriad), FoundationOne CDx (from Foundation Medicine), Tempus xT (from Tempus), and tests from Caris Molecular Intelligence.

RAD51 protein forms subnuclear complexes that are microscopically detectible as foci, which contain many of the enzymatic activities required for efficient repair of DSBs. RAD51 foci may be used as a surrogate marker of HRR functionality. Thus, reduced number of RAD51 foci and/or RAD51 signal is associated with homologous recombination deficiency.

A genomic scar (also referred to as a mutational scar) can be defined as a genomic aberration with a known origin, and may include aberrations such as LOH, TAI, LST, total number of somatic, synonymous, and non-synonymous coding mutations (Nmut). Genomic scar assays may be used to determine whether a cancer is an HRD cancer, as described in Waktins et al. (2014) Breast Cancer Res. 16(3):211, which is herein incorporated by reference. Alternatively or in addition, specific transcriptome abnormalities which create a unique transcriptomic signature may be determined/quantified using techniques such as gene expression, allele-specific expression, and alternative splicing from RNA-sequencing data, and used to determine whether a cancer is an HRD cancer.

Deficiencies in HR or HRR are known to be associated with sensitivity to platinum-based chemotherapeutic agents. Consequently, detecting and/or quantifying platinum sensitivity of cancerous and/or non-cancerous cells from an individual may be used as determine whether a cancer is an HRD cancer. Typically platinum sensitivity is assessed at the level of a clinical response, i.e. whether a cancer or cells thereof respond fully or partially to platinum chemotherapy assessed using normal clinical tests and/or parameters.

The present invention provides an assay comprising: measuring or quantifying a mutation or deficiency in one or more gene associated with HR or HRR, such as those described herein (e.g. BRCA1 and/or BRCA2) in a biological sample obtained from an individual with cancer; and comparing the measured or quantified amount of the one or more gene associated with HR or HRR, such as those described herein (e.g. BRCA1 and/or BRCA2) with a reference value, and if the one or more gene associated with HR or HRR, such as those described herein (e.g. BRCA1 and/or BRCA2) is mutated or deficient relative to the reference value, identifying the individual as having an increased probability of being responsive to treatment with an ADAR1 inhibitor.

Selecting Individuals with HRD Cancer for Treatment with an ADAR1 Inhibitor

The invention also provides a method of selecting an individual having cancer for treatment with an ADAR1 inhibitor. Said method typically comprises (a) determining whether the cancer is an HRD cancer; and (b) selecting the individual for treatment with the ADAR1 inhibitor where the cancer is an HRD cancer. Said method may further comprise providing an ADAR1 inhibitor suitable for administration to the individual, and may yet further comprise administering a therapeutically effective amount of the ADAR1 inhibitor to the individual. Determining whether the cancer is an HDR cancer may comprise the use of a sample comprising or consisting of cancerous and/or non-cancerous cells from the individual.

Any and all disclosure herein in relation to determining whether a cancer is an HRD cancer in the context of treating an individual with HRD cancer applies equally and without reservation to methods for screening or selecting an individual with HRD cancer for treatment according to the invention.

By way of non-limiting example, in a method of selecting an individual having HRD cancer for treatment with an ADAR1 inhibitor, determining whether the cancer is an HRD cancer may comprise: (a) determining whether the cancer is mutated or deficient in one or more gene associated with homologous recombination deficiency, such as those described herein (e.g. BRCA1 and/or BRCA2); (b) the use of a companion diagnostic for homologous recombination deficiency, such as those described herein; (c) detecting and/or quantifying RAD51 foci within the sample, wherein reduced RAD51 signal is associated with homologous recombination deficiency; (d) detecting and/or quantifying a homologous recombination deficiency defect, optionally a transcriptomic signature and/or mutational scar associated with homologous recombination deficiency; and/or I detecting and/or quantifying platinum sensitivity of the cancer, wherein platinum sensitivity is associated with homologous recombination deficiency. Any and all of the disclosure herein in relation to the determination of whether a cancer is an HRD cancer, e.g. in relation to particular genes associated with homologous recombination deficiency and/or techniques for determining the presence of a deficiency and/or mutation in one or more gene associated with homologous recombination deficiency, applies equally and without reservation to the selection methods of the invention.

Preferably the invention provides a method of selecting an individual having cancer for treatment with an ADAR1 inhibitor, the method comprising: (a) determining in a sample obtained from the individual whether the cancer is an HRD cancer; (b) selecting the individual for treatment with the ADAR1 inhibitor where the cancer is an HRD cancer; and optionally providing a ADAR1 inhibitor suitable for administration to the individual; wherein determining whether the cancer is an HRD cancer comprises determining whether the cancer is mutated or deficient in BRCA1 and/or BRCA2, preferably BRCA1.

Preferably the invention provides a method of selecting an individual having an HRD cancer for treatment with an ADAR1 inhibitor, wherein the cancer is breast cancer, ovarian cancer, pancreatic cancer, biliary tract cancer or prostate cancer.

### Methods of Screening for ADAR1 Inhibitors

The present invention also includes methods of screening that employ ADAR1 as a protein target for the screening of candidate compounds to find ADAR1 inhibitors. Accordingly, methods of screening may be carried out for identifying candidate agents that are capable of inhibiting ADAR1, for subsequent use of development as agents for the treatment of HRD cancer. Conveniently, this may be done in an assay buffer to help the components of the assay interact, and in a multiple well format to test a plurality of candidate agents. The activity of ADAR1 can then be determined in the presence and absence of the one or more candidate compounds to determine whether a given candidate is a ADAR1 inhibitor.

By way of example, the candidate agent may be a known inhibitor of one of the protein targets disclosed herein, an antibody, a peptide, a nucleic acid molecule or a small molecule (e.g. an organic or inorganic compound), typically of molecular weight of less than 500 Da. In some instances, the use of candidate agents that are small molecules is preferred. However, for any type of candidate agent, combinatorial library technology provides an efficient way of testing a potentially vast number of different substances for ability to modulate activity of a target protein. Such libraries and their use are known in the art. The present invention also specifically envisages screening candidate agents known for the treatment of other conditions, and especially other forms of cancer. This has the advantage that the patient or disease profile of known therapeutic agents might be expanded or modified using the screening techniques disclosed herein, or for therapeutic agents in development, patient or disease profiles established that are relevant for the treatment of HRD cancer.

Following identification of a candidate agent for further investigation, the agent in question may be tested to determine whether it is not lethal to normal cells or otherwise is suited to therapeutic use. Following these studies, the agent may be manufactured and/or used in the preparation of a medicament, pharmaceutical composition, or dosage form.

The development of lead agents or compounds from an initial hit in screening assays might be desirable where the agent in question is difficult or expensive to synthesise or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as a “pharmacophore”.

Once the pharmacophore has been found, its structure is modelled to according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process. In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetics found by this approach can then be screened to see whether they have the target property, or to what extent they exhibit it. Further optimisation or modification can then be carried out to arrive at one or more final mimetics for in vivo or clinical testing.

### Formulations, Compositions and Administration

The ADAR1 inhibitors herein for the treatment of HRD cancer may be administered alone, but it is generally preferable to provide them in pharmaceutical compositions that additionally comprise with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Examples of components of pharmaceutical compositions are provided in Remington's Pharmaceutical Sciences, 20th Edition, 2000, pub. Lippincott, Williams & Wilkins.

These compounds or derivatives of them may be used in the present invention for the treatment of HRD cancer.

As used herein “derivatives” of the ADAR1 inhibitors includes salts, coordination complexes, esters such as in vivo hydrolysable esters, free acids or bases, hydrates, prodrugs or lipids, coupling partners.

Salts of the ADAR1 inhibitors of the invention are preferably physiologically well tolerated and non-toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g., amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in an ADAR1 inhibitor and an appropriate carboxylic acid or alcohol reaction partner, using techniques well known in the art.

Derivatives include prodrugs of the ADAR1 inhibitors which are convertible in vivo or in vitro into an active ADAR1 inhibitor. Typically, at least one of the biological activities of an ADAR1 inhibitor will be reduced in the prodrug form of the ADAR1 inhibitor, and can be activated by conversion of the prodrug to release the ADAR1 inhibitor or a metabolite of it.

Other derivatives include coupling partners of the ADAR1 inhibitors in which an ADAR1 inhibitor is linked to a coupling partner, e.g. by being chemically coupled to the ADAR1 inhibitor or physically associated with it. Examples of coupling partners include a label or reporter molecule, a supporting substrate, a carrier or transport molecule, an effector, a drug, an antibody or an inhibitor. Coupling partners can be covalently linked to ADAR1 inhibitors of the invention via an appropriate functional group on the ADAR1 inhibitor, such as a hydroxyl group, a carboxyl group or an amino group. Other derivatives include formulating the ADAR1 inhibitors with liposomes.

The term “pharmaceutically acceptable” as used herein includes compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The ADAR1 inhibitors disclosed herein for the treatment of HRD cancer according to the present invention are preferably for administration to an individual in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, Lippincott, Williams & Wilkins. By way of non-limiting example, therapeutic efficacy and toxicity of the compound can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. It will be appreciated that the dosage range required depends on the precise nature of the ADAR1 inhibitor, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

An ADAR1 inhibitor may be administered alone or in combination with other treatments, either simultaneously or sequentially, dependent upon the condition to be treated.

Any two or more ADAR1 inhibitors of the invention may be administered separately, sequentially or simultaneously. The two or more ADAR1 inhibitors may be administered in the same or different compositions. In a preferred instance, the two or more ADAR1 inhibitors may be delivered in the same composition.

An ADAR1 inhibitor may be formulated using standard techniques and additional components as described herein. The formulations may conveniently be presented in unit dosage form and may be prepared by any methods well known in the art of pharmacy. Such methods include the step of bringing the ADAR1 inhibitor into association with a carrier which may constitute one or more accessory ingredients. In general, the formulations are prepared by uniformly and intimately bringing into association the ADAR1 inhibitor with liquid carriers or finely divided solid carriers or both, and then if necessary, shaping the product.

The ADAR1 inhibitors may be administered to an individual by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. It may be desired to direct the compositions of the present invention (as described above) to the tissue or organ comprising a tumour to be treated according to the invention.

Formulations suitable for oral administration (e.g., by ingestion) may be presented as discrete units such as capsules, cachets or tablets, each containing a predetermined amount of the ADAR1 inhibitor; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. By way of non-limiting example, the concentration of the ADAR1 inhibitor in the solution is from about 1 ng/ml to about 10 mg/ml, for example from about 10 ng/ml to about 1 mg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the ADAR1 inhibitor to blood components or one or more organs.

Liquid compositions may be sterilised by filtration through a sterile filter using aseptic techniques before filling into suitable sterile containers (e.g. vials or ampoules) and sealing. Alternatively, if solution stability is adequate, the solution in its sealed containers may be sterilised by autoclaving. Additives such as preservative or bactericidal, suspending or emulsifying agents and or local anaesthetic agents may be dissolved in the vehicle.

Solid formulations (e.g. dry powders), which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then lyophilised and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

The composition may be in lyophilized form, in which case it may include a stabilizer, such as bovine serum albumin (BSA). In some embodiments, it may be desirable to formulate the composition with a preservative, such as thiomersal or sodium azide, to facilitate long-term storage.

Administration in accordance with the present invention may take advantage of a variety of delivery technologies including microparticle encapsulation, viral delivery systems or high-pressure aerosol impingement.

ADAR1 inhibitors may be used in combination with one or more further anti-cancer therapies according to the invention. In particular, ADAR1 inhibitors may be used in combination with (i) one or more further chemotherapeutic agent; (ii) one or more immunotherapeutic agent; and/or (iii) radiotherapy, or a combination thereof.

Thus, ADAR1 inhibitors and compositions comprising ADAR1 inhibitors disclosed herein for the treatment of HRD cancer may be used in the methods described herein in combination with standard chemotherapeutic regimes and/or in conjunction with radiotherapy. As radiotherapy also leads to DNA strand breaks, causing severe DNA damage and leading to cell death, the combination of radiotherapy with ADAR1 inhibitors offers the potential to lead to formation of double strand breaks from the single-strand breaks generated by the radiotherapy in tumour tissue. This combination could therefore lead to either more powerful therapy with the same radiation dose or similarly powerful therapy with a lower radiation dose, potentially avoiding some of the side effects with radiotherapy.

By way of example, additional agents that might be employed in combination with the use of ADAR1 inhibitors as disclosed herein include one or more spliceosomal inhibitors, for example agents that target components of the spliceosome, such as SF3B1, e.g. using a SF3BI inhibitor, particularly small molecule inhibitors of SF3B1.

Alternatively or in addition, ADAR1 inhibitors may be used in combination with one or more chemotherapeutic agent according to the invention. Non-limiting examples of chemotherapeutic agents which may be used in combination with one or more ADAR1 inhibitor include Amsacrine (Amsidine), Bevacizumab (Avastin), Bleomycin, Busulfan, Capecitabine (Xeloda), Carboplatin, Carmustine (BCNU), Chlorambucil (Leukeran), Cisplatin, Cladribine (Leustat), Clofarabine (Evoltra), Crisantaspase (Erwinase), Cyclophosphamide, Cytarabine (ARA-C), Dacarbazine (DTIC), Dactinomycin (Actinomycin D),Daunorubicin, Docetaxel (Taxotere), Doxorubicin, Epirubicin, Etoposide (Vepesid, VP-16), Fludarabine (Fludara), Fluorouracil (5-FU), Gemcitabine (Gemzar), Hydroxyurea (Hydroxycarbamide, Hydrea), Idarubicin (Zavedos), Ifosfamide (Mitoxana), Irinotecan (CPT-11, Campto), Leucovorin (folinic acid), Liposomal doxorubicin (Caelyx, Myocet), Liposomal daunorubicin (DaunoXome®) Lomustine, Melphalan, Mercaptopurine, Mesna, Methotrexate, Mitomycin, Mitoxantrone, Oxaliplatin (Eloxatin), Paclitaxel (Taxol), Pemetrexed (Alimta), Pentostatin (Nipent), Procarbazine, Raltitrexed (Tomudex®), Streptozocin (Zanosar®), Tegafur-uracil (Uftoral), Temozolomide (Temodal), Teniposide (Vumon), Thiotepa, Tioguanine (6-TG) (Lanvis), Topotecan (Hycamtin), Treosulfan, Vinblastine (Velbe), Vincristine (Oncovin), Vindesine (Eldisine) or Vinorelbine (Navelbine), or the PARP inhibitors (Olaparib (Lynparza), Rucaparib, Niraparib, Veliparib and Talazoparib), ATR inhibitors, PolQ inhibitors, or any other DNA repair inhibitor.

Alternatively or in addition, ADAR1 inhibitors may be used in combination with one or more immunotherapeutic agent, such as an immune checkpoint inhibitor, monoclonal antibody (including bi-, tri- and multi-specific antibodies), cytokine, cell therapy, cancer vaccines, oncolytic viruses, antisense oligodeoxynucleotides, antibody-drug conjugates and/or any modulator of the cytosolic nucleic acid sensing pathways (e.g. agonist of the cGAS/stimulator of interferon genes (STING) pathway, i.e. a STING agonist).

Preferably, an ADAR1 inhibitor of the invention may be used in combination with one or more anti-cancer therapy for HRD cancer, for example platinum-based chemotherapeutics, PARP inhibitors, PolQ (Pole) inhibitors, RS-response kinase ataxia telangiectasia and Rad3-related protein (ATR) inhibitors and/or VEGF inhibitors (e.g. anti-VEGF monoclonal antibodies and tyrosine kinase inhibitors). Non-limiting examples of platinum-based chemotherapeutics include cisplatin, carboplatin, oxaliplatin and nedaplatin. Non-limiting examples of PARP inhibitors include niraparib (Zejula), olaparib (Lynparza), talazoparib (Talzenna) and rucaparib (Rubraca). Non-limiting examples of POLQ inhibitors include novobiocin, ART4215, ART0380 and ART558. Non-limiting examples of ATR inhibitors include AZD6738, M6620 (VX-970), BAY1895344, and M4344 (VX-803). Non-limiting examples of VEGF inhibitors include bevacizumab, aflibercept, ramucirumab, axitinib, cabozantinib, lapatinib, lenvatinib, pazopanib, ponatinib, regorafenib, sorafenib, sunitinib, and vandetanib.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In some cases after an initial administration of an ADAR1 inhibitor, a subsequent administration of an ADAR1 inhibitor may be performed. The administration may, for instance, be at least one week, two weeks, three weeks, four weeks, six weeks, two months, four moths, six months or more after the initial administration. The ADAR1 inhibitor may, for instance, be administered at intervals dictated by when the effects of the previous administration are decreasing, and when an additional administration will not exceed the therapeutic window.

By way of non-limiting example, for a small molecule ADAR1 inhibitor, a suitable dose may be in the range of about 100 mg to about 250 mg per kilogram body weight of the subject per day. Where the ADAR1 inhibitor is a salt, an ester, prodrug, or the like, the amount administered is calculated on the basis of the parent ADAR1 inhibitor, and so the actual weight to be used is increased proportionately.

The invention also provides a kit comprising a (solid or liquid) composition as described herein and instructions for therapeutic administration of said ADAR1 inhibitor to an individual in need thereof. More precisely, the invention relates to a kit comprising one or more ADAR1 inhibitor of the invention, or a pharmaceutical composition comprising one or more ADAR inhibitor of the invention, and instructions for therapeutic administration of said one or more ADAR1 inhibitor to an individual in need thereof. The one or more ADAR1 inhibitor may be provided in any suitable composition or formulation, such as those described herein. By way of non-limiting example, the one or more ADAR1 inhibitor may be provided in lyophilised form. As used herein, the term “instructions” refers to a publication, a recording, a diagram, or any other medium of expression which can be used to communicate how to perform a method or use of the invention, such as therapeutic or cosmetic administration of said composition to an individual in need thereof Said instructions can, for example, be affixed to a container which comprises said composition or said kit.

SEQUENCE INFORMATION
NM_001025107.3
SEQ ID NO: 1
ATTGATTCCCGACTGAAGGTAGAGAAGGCTACGTGGTGGGGGAGGGTGGGGGGAGGGTCGCGGCC
GCACTGGCAGTCTCCGGGTGTCCGGCCGTGTCCCGAGGAAGTGCAAGACCCGGGGTATTCCCTCA
GCGGATACTACACCCATCCATTTCAAGGCTATGAGCACAGACAGCTCAGGTACCAGCAGCCTGGG
CCAGGATCTTCCCCCAGTAGTTTCCTGCTTAAGCAAATAGAATTTCTCAAGGGGCAGCTCCCAGA
AGCACCGGTGATTGGAAAGCAGACACCGTCACTGCCACCTTCCCTCCCAGGACTCCGGCCAAGGT
TTCCAGTACTACTTGCCTCCAGTACCAGAGGCAGGCAAGTGGACATCAGGGGTGTCCCCAGGGGC
GTGCATCTCAGAAGTCAGGGGCTCCAGAGAGGGTTCCAGCATCCTTCACCACGTGGCAGGAGTCT
GCCACAGAGAGGTGTTGATTGCCTTTCCTCACATTTCCAGGAACTGAGTATCTACCAAGATCAGG
AACAAAGGATCTTAAAGTTCCTGGAAGAGCTTGGGGAAGGGAAGGCCACCACAGCACATGATCTG
TCTGGGAAACTTGGGACTCCGAAGAAAGAAATCAATCGAGTTTTATACTCCCTGGCAAAGAAGGG
CAAGCTACAGAAAGAGGCAGGAACACCCCCTTTGTGGAAAATCGCGGTCTCCACTCAGGCTTGGA
ACCAGCACAGCGGAGTGGTAAGACCAGACGGTCATAGCCAAGGAGCCCCAAACTCAGACCCGAGT
TTGGAACCGGAAGACAGAAACTCCACATCTGTCTCAGAAGATCTTCTTGAGCCTTTTATTGCAGT
CTCAGCTCAGGCTTGGAACCAGCACAGCGGAGTGGTAAGACCAGACAGTCATAGCCAAGGATCCC
CAAACTCAGACCCAGGTTTGGAACCTGAAGACAGCAACTCCACATCTGCCTTGGAAGATCCTCTT
GAGTTTTTAGACATGGCCGAGATCAAGGAGAAAATCTGCGACTATCTCTTCAATGTGTCTGACTC
CTCTGCCCTGAATTTGGCTAAAAATATTGGCCTTACCAAGGCCCGAGATATAAATGCTGTGCTAA
TTGACATGGAAAGGCAGGGGGATGTCTATAGACAAGGGACAACCCCTCCCATATGGCATTTGACA
GACAAGAAGCGAGAGAGGATGCAAATCAAGAGAAATACGAACAGTGTTCCTGAAACCGCTCCAGC
TGCAATCCCTGAGACCAAAAGAAACGCAGAGTTCCTCACCTGTAATATACCCACATCAAATGCCT
CAAATAACATGGTAACCACAGAAAAAGTGGAGAATGGGCAGGAACCTGTCATAAAGTTAGAAAAC
AGGCAAGAGGCCAGACCAGAACCAGCAAGACTGAAACCACCTGTTCATTACAATGGCCCCTCAAA
AGCAGGGTATGTTGACTTTGAAAATGGCCAGTGGGCCACAGATGACATCCCAGATGACTTGAATA
GTATCCGCGCAGCACCAGGTGAGTTTCGAGCCATCATGGAGATGCCCTCCTTCTACAGTCATGGC
TTGCCACGGTGTTCACCCTACAAGAAACTGACAGAGTGCCAGCTGAAGAACCCCATCAGCGGGCT
GTTAGAATATGCCCAGTTCGCTAGTCAAACCTGTGAGTTCAACATGATAGAGCAGAGTGGACCAC
CCCATGAACCTCGATTTAAATTCCAGGTTGTCATCAATGGCCGAGAGTTTCCCCCAGCTGAAGCT
GGAAGCAAGAAAGTGGCCAAGCAGGATGCAGCTATGAAAGCCATGACAATTCTGCTAGAGGAAGC
CAAAGCCAAGGACAGTGGAAAATCAGAAGAATCATCCCACTATTCCACAGAGAAAGAATCAGAGA
AGACTGCAGAGTCCCAGACCCCCACCCCTTCAGCCACATCCTTCTTTTCTGGGAAGAGCCCCGTC
ACCACACTGCTTGAGTGTATGCACAAATTGGGGAACTCCTGCGAATTCCGTCTCCTGTCCAAAGA
AGGCCCTGCCCATGAACCCAAGTTCCAATACTGTGTTGCAGTGGGAGCCCAAACTTTCCCCAGTG
TGAGTGCTCCCAGCAAGAAAGTGGCAAAGCAGATGGCCGCAGAGGAAGCCATGAAGGCCCTGCAT
GGGGAGGCGACCAACTCCATGGCTTCTGATAACCAGCCTGAAGGTATGATCTCAGAGTCACTTGA
TAACTTGGAATCCATGATGCCCAACAAGGTCAGGAAGATTGGCGAGCTCGTGAGATACCTGAACA
CCAACCCTGTGGGTGGCCTTTTGGAGTACGCCCGCTCCCATGGCTTTGCTGCTGAATTCAAGTTG
GTCGACCAGTCCGGACCTCCTCACGAGCCCAAGTTCGTTTACCAAGCAAAAGTTGGGGGTCGCTG
GTTCCCAGCCGTCTGCGCACACAGCAAGAAGCAAGGCAAGCAGGAAGCAGCAGATGCGGCTCTCC
GTGTCTTGATTGGGGAGAACGAGAAGGCAGAACGCATGGGTTTCACAGAGGTAACCCCAGTGACA
GGGGCCAGTCTCAGAAGAACTATGCTCCTCCTCTCAAGGTCCCCAGAAGCACAGCCAAAGACACT
CCCTCTCACTGGCAGCACCTTCCATGACCAGATAGCCATGCTGAGCCACCGGTGCTTCAACACTC
TGACTAACAGCTTCCAGCCCTCCTTGCTCGGCCGCAAGATTCTGGCCGCCATCATTATGAAAAAA
GACTCTGAGGACATGGGTGTCGTCGTCAGCTTGGGAACAGGGAATCGCTGTGTGAAAGGAGATTC
TCTCAGCCTAAAAGGAGAAACTGTCAATGACTGCCATGCAGAAATAATCTCCCGGAGAGGCTTCA
TCAGGTTTCTCTACAGTGAGTTAATGAAATACAACTCCCAGACTGCGAAGGATAGTATATTTGAA
CCTGCTAAGGGAGGAGAAAAGCTCCAAATAAAAAAGACTGTGTCATTCCATCTGTATATCAGCAC
TGCTCCGTGTGGAGATGGCGCCCTCTTTGACAAGTCCTGCAGCGACCGTGCTATGGAAAGCACAG
AATCCCGCCACTACCCTGTCTTCGAGAATCCCAAACAAGGAAAGCTCCGCACCAAGGTGGAGAAC
GGAGAAGGCACAATCCCTGTGGAATCCAGTGACATTGTGCCTACGTGGGATGGCATTCGGCTCGG
GGAGAGACTCCGTACCATGTCCTGTAGTGACAAAATCCTACGCTGGAACGTGCTGGGCCTGCAAG
GGGCACTGTTGACCCACTTCCTGCAGCCCATTTATCTCAAATCTGTCACATTGGGTTACCTTTTC
AGCCAAGGGCATCTGACCCGTGCTATTTGCTGTCGTGTGACAAGAGATGGGAGTGCATTTGAGGA
TGGACTACGACATCCCTTTATTGTCAACCACCCCAAGGTTGGCAGAGTCAGCATATATGATTCCA
AAAGGCAATCCGGGAAGACTAAGGAGACAAGCGTCAACTGGTGTCTGGCTGATGGCTATGACCTG
GAGATCCTGGACGGTACCAGAGGCACTGTGGATGGGCCACGGAATGAATTGTCCCGGGTCTCCAA
AAAGAACATTTTTCTTCTATTTAAGAAGCTCTGCTCCTTCCGTTACCGCAGGGATCTACTGAGAC
TCTCCTATGGTGAGGCCAAGAAAGCTGCCCGTGACTACGAGACGGCCAAGAACTACTTCAAAAAA
GGCCTGAAGGATATGGGCTATGGGAACTGGATTAGCAAACCCCAGGAGGAAAAGAACTTTTATCT
CTGCCCAGTATAGTATGCTCCAGTGACAGATGGATTAGGGTGTGTCATACTAGGGTGTGAGAGAG
GTAGGTCGTAGCATTCCTCATCACATGGTCAGGGGATTTTTTTTTCTCCTTTTTTTTTCTTTTTA
AGCCATAATTGGTGATACTGAAAACTTTGGGTTCCCATTTATCCTGCTTTCTTTGGGATTGCTAG
GCAAGGTCTGGCCAGGCCCCCCTTTTTTCCCCCAAGTGAAGAGGCAGAAACCTAAGAAGTTATCT
TTTCTTTCTACCCAAAGCATACATAGTCACTGAGCACCTGCGGTCCATTTCCTCTTAAAAGTTTT
GTTTTGATTTGTTTCCATTTCCTTTCCCTTTGTGTTTGCTACACTGACCTCTTGCGGTCTTGATT
AGGTTTCAGTCAACTCTGGATCATGTCAGGGACTGATAATTTCATTTGTGGATTACGCAGACCCC
TCTACTTCCCCTCTTTCCCTTCTGAGATTCTTTCCTTGTGATCTGAATGTCTCCTTTTCCCCCTC
AGAGGGCAAAGAGGTGAACATAAAGGATTTGGTGAAACATTTGTAAGGGTAGGAGTTGAAAACTG
CAGTTCCCAGTGCCACGGAAGTGTGATTGGAGCCTGCAGATAATGCCCAGCCATCCTCCCATCCT
GCACTTTAGCCAGCTGCAGGGCGGGCAAGGCAAGGAAAGCTGCTTCCCTGGAAGTGTATCACTTT
CTCCGGCAGCTGGGAAGTCTAGAACCAGCCAGACTGGGTTAAGGGAGCTGCTCAAGCAATAGCAG
AGGTTTCACCCGGCAGGATGACACAGACCACTTCCCAGGGAGCACGGGCATGCCTTGGAATATTG
CCAAGCTTCCAGCTGCCTCTTCTCCTAAAGCATTCCTAGGAATATTTTCCCCGCCAATGCTGGGC
GTACACCCTAGCCAACGGGACAAATCCTAGAGGGTATAAAATCATCTCTGCTCAGATAATCATGA
CTTAGCAAGAATAAGGGCAAAAAATCCTGTTGGCTTAACGTCACTGTTCCACCCGGTGTAATATC
TCTCATGACAGTGACACCAAGGGAAGTTGACTAAGTCACATGTAAATTAGGAGTGTTTTAAAGAA
TGCCATAGATGTTGATTCTTAACTGCTACAGATAACCTGTAATTGAGCAGATTTAAAATTCAGGC
ATACTTTTCCATTTATCCAAGTGCTTTCATTTTTCCAGATGGCTTCAGAAGTAGGCTCGTGGGCA
GGGCGCAGACCTGATCTTTATAGGGTTGACATAGAAAGCAGTAGTTGTGGGTGAAAGGGCAGGTT
GTCTTCAAACTCTGTGAGGTAGAATCCTTTGTCTATACCTCCATGAACATTGACTCGTGTGTTCA
GAGCCTTTGGCCTCTCTGTGGAGTCTGGCTCTCTGGCTCCTGTGCATTCTTTGAATAGTCACTCG
TAAAAACTGTCAGTGCTTGAAACTGTTTCCTTTACTCATGTTGAAGGGACTTTGTTGGCTTTTAG
AGTGTTGGTCATGACTCCAAGAGCAGAGCAGGGAAGAGCCCAAGCATAGACTTGGTGCCGTGGTG
ATGGCTGCAGTCCAGTTTTGTGATGCTGCTTTTACGTGTCCCTCGATAACAGTCAGCTAGACACA
CTCAGGAGGACTACTGAGGCTCTGCGACCTTCAGGAGCTGAGCCTGCCTCTCTCCTTTAGATGAC
AGACCTTCATCTGGGAACGTGCTGAGCCAGCACCCTCAGATGATTTCCCTCCAAACTGCTGACTA
GGTCATCCTCTGTCTGGTAGAGACATTCACATCTTTGCTTTTATTCTATGCTCTCTGTACTTTTG
ACCAAAAATTGACCAAAGTAAGAAAATGCAAGTTCTAAAAATAGACTAAGGATGCCTTTGCAGAA
CACCAAAGCATCCCAAGGAACTGGTAGGGAAGTGGCGCCTGTCTCCTGGAGTGGAAGAGGCCTGC
TCCCTGGCTCTGGGTCTGCTGGGGGCACAGTAAATCAGTCTTGGCACCCACATCCAGGGCAGAGA
GGTCTGTGGTTCTCAGCATCAGAAGGCAGCGCAGCCCCTCTCCTCTTCAGGCTACAGGGTTGTCA
CCTGCTGAGTCCTCAGGTTGTTTGGCCTCTCTGGTCCATCTTGGGCATTAGGTTCTCCAGCAGAG
CTCTGGCCAGCTGCCTCTTCTTTAACTGGGAACACAGGCTCTCACAAGATCAGAACCCCCACTCA
CCCCCAAGATCTTATCTAGCAAGCCTGTAGTATTCAGTTTCTGTTGTAGGAAGAGAGCGAGGCAT
CCCTGAATTCCACGCATCTGCTGGAAACGAGCCGTGTCAGATCGCACATCCCTGCGCCCCCATGC
CCCTCTGAGTCACACAGGACAGAGGAGGCAGAGCTTCTGCCCACTGTTATCTTCACTTTCTTTGT
CCAGTCTTTTGTTTTTAATAAGCAGTGACCCTCCCTACTCTTCTTTTTAATGATTTTTGTAGTTG
ATTTGTCTGAACTGTGGCTACTGTGCATTCCTTGAATAATCACTTGTAAAAATTGTCAGTGCTTG
AAGCTGTTTCCTTTACTCACATTGAAGGGACTTCGTTGGTTTTTTGGAGTCTTGGTTGTGACTCC
AAGAGCAGAGTGAGGAAGACCCCCAAGCATAGACTCGGGTACTGTGATGATGGCTGCAGTCCAGT
TTTATGATTCTGCTTTTATGTGTCCCTTGATAACAGTGACTTAACAATATACATTCCTCATAAAT
AAAAAAAAAACAAGAATCTGAATTCTTAGAAA
NP_001020278.1; NP_001351975.1; NP_001180424.1; NP_001351976.1;
NP_001351977.1
SEQ ID NO: 2
MAEIKEKICDYLFNVSDSSALNLAKNIGLTKARDINAVLIDMERQGDVYRQGTTPPIWHLTDKKR
ERMQIKRNTNSVPETAPAAIPETKRNAEFLTCNIPTSNASNNMVTTEKVENGQEPVIKLENRQEA
RPEPARLKPPVHYNGPSKAGYVDFENGQWATDDIPDDLNSIRAAPGEFRAIMEMPSFYSHGLPRC
SPYKKLTECQLKNPISGLLEYAQFASQTCEFNMIEQSGPPHEPRFKFQVVINGREFPPAEAGSKK
VAKQDAAMKAMTILLEEAKAKDSGKSEESSHYSTEKESEKTAESQTPTPSATSFFSGKSPVTTLL
ECMHKLGNSCEFRLLSKEGPAHEPKFQYCVAVGAQTFPSVSAPSKKVAKQMAAEEAMKALHGEAT
NSMASDNQPEGMISESLDNLESMMPNKVRKIGELVRYLNTNPVGGLLEYARSHGFAAEFKLVDQS
GPPHEPKFVYQAKVGGRWFPAVCAHSKKQGKQEAADAALRVLIGENEKAERMGFTEVTPVTGASL
RRTMLLLSRSPEAQPKTLPLTGSTFHDQIAMLSHRCENTLTNSFQPSLLGRKILAAIIMKKDSED
MGVVVSLGTGNRCVKGDSLSLKGETVNDCHAEIISRRGFIRFLYSELMKYNSQTAKDSIFEPAKG
GEKLQIKKTVSFHLYISTAPCGDGALEDKSCSDRAMESTESRHYPVFENPKQGKLRTKVENGEGT
IPVESSDIVPTWDGIRLGERLRTMSCSDKILRWNVLGLQGALLTHFLQPIYLKSVTLGYLFSQGH
LTRAICCRVTRDGSAFEDGLRHPFIVNHPKVGRVSIYDSKRQSGKTKETSVNWCLADGYDLEILD
GTRGTVDGPRNELSRVSKKNIFLLFKKLCSFRYRRDLLRLSYGEAKKAARDYETAKNYFKKGLKD
MGYGNWISKPQEEKNFYLCPV
NM_001365046.1
SEQ ID NO: 3
TGATGTCACCAATCTGCGACCAGACCATTGATTCCCGACTGAAGGGGTATTCCCTCAGCGGATAC
TACACCCATCCATTTCAAGGCTATGAGCACAGACAGCTCAGGTACCAGCAGCCTGGGCCAGGATC
TTCCCCCAGTAGTTTCCTGCTTAAGCAAATAGAATTTCTCAAGGGGCAGCTCCCAGAAGCACCGG
TGATTGGAAAGCAGACACCGTCACTGCCACCTTCCCTCCCAGGACTCCGGCCAAGGTTTCCAGTA
CTACTTGCCTCCAGTACCAGAGGCAGGAACTGAGTATCTACCAAGATCAGGAACAAAGGATCTTA
AAGTTCCTGGAAGAGCTTGGGGAAGGGAAGGCCACCACAGCACATGATCTGTCTGGGAAACTTGG
GACTCCGAAGAAAGAAATCAATCGAGTTTTATACTCCCTGGCAAAGAAGGGCAAGCTACAGAAAG
AGGCAGGAACACCCCCTTTGTGGAAAATCGCGGTCTCCACTCAGGCTTGGAACCAGCACAGCGGA
GTGGTAAGACCAGACGGTCATAGCCAAGGAGCCCCAAACTCAGACCCGAGTTTGGAACCGGAAGA
CAGAAACTCCACATCTGTCTCAGAAGATCTTCTTGAGCCTTTTATTGCAGTCTCAGCTCAGGCTT
GGAACCAGCACAGCGGAGTGGTAAGACCAGACAGTCATAGCCAAGGATCCCCAAACTCAGACCCA
GGTTTGGAACCTGAAGACAGCAACTCCACATCTGCCTTGGAAGATCCTCTTGAGTTTTTAGACAT
GGCCGAGATCAAGGAGAAAATCTGCGACTATCTCTTCAATGTGTCTGACTCCTCTGCCCTGAATT
TGGCTAAAAATATTGGCCTTACCAAGGCCCGAGATATAAATGCTGTGCTAATTGACATGGAAAGG
CAGGGGGATGTCTATAGACAAGGGACAACCCCTCCCATATGGCATTTGACAGACAAGAAGCGAGA
GAGGATGCAAATCAAGAGAAATACGAACAGTGTTCCTGAAACCGCTCCAGCTGCAATCCCTGAGA
CCAAAAGAAACGCAGAGTTCCTCACCTGTAATATACCCACATCAAATGCCTCAAATAACATGGTA
ACCACAGAAAAAGTGGAGAATGGGCAGGAACCTGTCATAAAGTTAGAAAACAGGCAAGAGGCCAG
ACCAGAACCAGCAAGACTGAAACCACCTGTTCATTACAATGGCCCCTCAAAAGCAGGGTATGTTG
ACTTTGAAAATGGCCAGTGGGCCACAGATGACATCCCAGATGACTTGAATAGTATCCGCGCAGCA
CCAGGTGAGTTTCGAGCCATCATGGAGATGCCCTCCTTCTACAGTCATGGCTTGCCACGGTGTTC
ACCCTACAAGAAACTGACAGAGTGCCAGCTGAAGAACCCCATCAGCGGGCTGTTAGAATATGCCC
AGTTCGCTAGTCAAACCTGTGAGTTCAACATGATAGAGCAGAGTGGACCACCCCATGAACCTCGA
TTTAAATTCCAGGTTGTCATCAATGGCCGAGAGTTTCCCCCAGCTGAAGCTGGAAGCAAGAAAGT
GGCCAAGCAGGATGCAGCTATGAAAGCCATGACAATTCTGCTAGAGGAAGCCAAAGCCAAGGACA
GTGGAAAATCAGAAGAATCATCCCACTATTCCACAGAGAAAGAATCAGAGAAGACTGCAGAGTCC
CAGACCCCCACCCCTTCAGCCACATCCTTCTTTTCTGGGAAGAGCCCCGTCACCACACTGCTTGA
GTGTATGCACAAATTGGGGAACTCCTGCGAATTCCGTCTCCTGTCCAAAGAAGGCCCTGCCCATG
AACCCAAGTTCCAATACTGTGTTGCAGTGGGAGCCCAAACTTTCCCCAGTGTGAGTGCTCCCAGC
AAGAAAGTGGCAAAGCAGATGGCCGCAGAGGAAGCCATGAAGGCCCTGCATGGGGAGGCGACCAA
CTCCATGGCTTCTGATAACCAGCCTGAAGGTATGATCTCAGAGTCACTTGATAACTTGGAATCCA
TGATGCCCAACAAGGTCAGGAAGATTGGCGAGCTCGTGAGATACCTGAACACCAACCCTGTGGGT
GGCCTTTTGGAGTACGCCCGCTCCCATGGCTTTGCTGCTGAATTCAAGTTGGTCGACCAGTCCGG
ACCTCCTCACGAGCCCAAGTTCGTTTACCAAGCAAAAGTTGGGGGTCGCTGGTTCCCAGCCGTCT
GCGCACACAGCAAGAAGCAAGGCAAGCAGGAAGCAGCAGATGCGGCTCTCCGTGTCTTGATTGGG
GAGAACGAGAAGGCAGAACGCATGGGTTTCACAGAGGTAACCCCAGTGACAGGGGCCAGTCTCAG
AAGAACTATGCTCCTCCTCTCAAGGTCCCCAGAAGCACAGCCAAAGACACTCCCTCTCACTGGCA
GCACCTTCCATGACCAGATAGCCATGCTGAGCCACCGGTGCTTCAACACTCTGACTAACAGCTTC
CAGCCCTCCTTGCTCGGCCGCAAGATTCTGGCCGCCATCATTATGAAAAAAGACTCTGAGGACAT
GGGTGTCGTCGTCAGCTTGGGAACAGGGAATCGCTGTGTGAAAGGAGATTCTCTCAGCCTAAAAG
GAGAAACTGTCAATGACTGCCATGCAGAAATAATCTCCCGGAGAGGCTTCATCAGGTTTCTCTAC
AGTGAGTTAATGAAATACAACTCCCAGACTGCGAAGGATAGTATATTTGAACCTGCTAAGGGAGG
AGAAAAGCTCCAAATAAAAAAGACTGTGTCATTCCATCTGTATATCAGCACTGCTCCGTGTGGAG
ATGGCGCCCTCTTTGACAAGTCCTGCAGCGACCGTGCTATGGAAAGCACAGAATCCCGCCACTAC
CCTGTCTTCGAGAATCCCAAACAAGGAAAGCTCCGCACCAAGGTGGAGAACGGAGAAGGCACAAT
CCCTGTGGAATCCAGTGACATTGTGCCTACGTGGGATGGCATTCGGCTCGGGGAGAGACTCCGTA
CCATGTCCTGTAGTGACAAAATCCTACGCTGGAACGTGCTGGGCCTGCAAGGGGCACTGTTGACC
CACTTCCTGCAGCCCATTTATCTCAAATCTGTCACATTGGGTTACCTTTTCAGCCAAGGGCATCT
GACCCGTGCTATTTGCTGTCGTGTGACAAGAGATGGGAGTGCATTTGAGGATGGACTACGACATC
CCTTTATTGTCAACCACCCCAAGGTTGGCAGAGTCAGCATATATGATTCCAAAAGGCAATCCGGG
AAGACTAAGGAGACAAGCGTCAACTGGTGTCTGGCTGATGGCTATGACCTGGAGATCCTGGACGG
TACCAGAGGCACTGTGGATGGGCCACGGAATGAATTGTCCCGGGTCTCCAAAAAGAACATTTTTC
TTCTATTTAAGAAGCTCTGCTCCTTCCGTTACCGCAGGGATCTACTGAGACTCTCCTATGGTGAG
GCCAAGAAAGCTGCCCGTGACTACGAGACGGCCAAGAACTACTTCAAAAAAGGCCTGAAGGATAT
GGGCTATGGGAACTGGATTAGCAAACCCCAGGAGGAAAAGAACTTTTATCTCTGCCCAGTATAGT
ATGCTCCAGTGACAGATGGATTAGGGTGTGTCATACTAGGGTGTGAGAGAGGTAGGTCGTAGCAT
TCCTCATCACATGGTCAGGGGATTTTTTTTTCTCCTTTTTTTTTCTTTTTAAGCCATAATTGGTG
ATACTGAAAACTTTGGGTTCCCATTTATCCTGCTTTCTTTGGGATTGCTAGGCAAGGTCTGGCCA
GGCCCCCCTTTTTTCCCCCAAGTGAAGAGGCAGAAACCTAAGAAGTTATCTTTTCTTTCTACCCA
AAGCATACATAGTCACTGAGCACCTGCGGTCCATTTCCTCTTAAAAGTTTTGTTTTGATTTGTTT
CCATTTCCTTTCCCTTTGTGTTTGCTACACTGACCTCTTGCGGTCTTGATTAGGTTTCAGTCAAC
TCTGGATCATGTCAGGGACTGATAATTTCATTTGTGGATTACGCAGACCCCTCTACTTCCCCTCT
TTCCCTTCTGAGATTCTTTCCTTGTGATCTGAATGTCTCCTTTTCCCCCTCAGAGGGCAAAGAGG
TGAACATAAAGGATTTGGTGAAACATTTGTAAGGGTAGGAGTTGAAAACTGCAGTTCCCAGTGCC
ACGGAAGTGTGATTGGAGCCTGCAGATAATGCCCAGCCATCCTCCCATCCTGCACTTTAGCCAGC
TGCAGGGCGGGCAAGGCAAGGAAAGCTGCTTCCCTGGAAGTGTATCACTTTCTCCGGCAGCTGGG
AAGTCTAGAACCAGCCAGACTGGGTTAAGGGAGCTGCTCAAGCAATAGCAGAGGTTTCACCCGGC
AGGATGACACAGACCACTTCCCAGGGAGCACGGGCATGCCTTGGAATATTGCCAAGCTTCCAGCT
GCCTCTTCTCCTAAAGCATTCCTAGGAATATTTTCCCCGCCAATGCTGGGCGTACACCCTAGCCA
ACGGGACAAATCCTAGAGGGTATAAAATCATCTCTGCTCAGATAATCATGACTTAGCAAGAATAA
GGGCAAAAAATCCTGTTGGCTTAACGTCACTGTTCCACCCGGTGTAATATCTCTCATGACAGTGA
CACCAAGGGAAGTTGACTAAGTCACATGTAAATTAGGAGTGTTTTAAAGAATGCCATAGATGTTG
ATTCTTAACTGCTACAGATAACCTGTAATTGAGCAGATTTAAAATTCAGGCATACTTTTCCATTT
ATCCAAGTGCTTTCATTTTTCCAGATGGCTTCAGAAGTAGGCTCGTGGGCAGGGCGCAGACCTGA
TCTTTATAGGGTTGACATAGAAAGCAGTAGTTGTGGGTGAAAGGGCAGGTTGTCTTCAAACTCTG
TGAGGTAGAATCCTTTGTCTATACCTCCATGAACATTGACTCGTGTGTTCAGAGCCTTTGGCCTC
TCTGTGGAGTCTGGCTCTCTGGCTCCTGTGCATTCTTTGAATAGTCACTCGTAAAAACTGTCAGT
GCTTGAAACTGTTTCCTTTACTCATGTTGAAGGGACTTTGTTGGCTTTTAGAGTGTTGGTCATGA
CTCCAAGAGCAGAGCAGGGAAGAGCCCAAGCATAGACTTGGTGCCGTGGTGATGGCTGCAGTCCA
GTTTTGTGATGCTGCTTTTACGTGTCCCTCGATAACAGTCAGCTAGACACACTCAGGAGGACTAC
TGAGGCTCTGCGACCTTCAGGAGCTGAGCCTGCCTCTCTCCTTTAGATGACAGACCTTCATCTGG
GAACGTGCTGAGCCAGCACCCTCAGATGATTTCCCTCCAAACTGCTGACTAGGTCATCCTCTGTC
TGGTAGAGACATTCACATCTTTGCTTTTATTCTATGCTCTCTGTACTTTTGACCAAAAATTGACC
AAAGTAAGAAAATGCAAGTTCTAAAAATAGACTAAGGATGCCTTTGCAGAACACCAAAGCATCCC
AAGGAACTGGTAGGGAAGTGGCGCCTGTCTCCTGGAGTGGAAGAGGCCTGCTCCCTGGCTCTGGG
TCTGCTGGGGGCACAGTAAATCAGTCTTGGCACCCACATCCAGGGCAGAGAGGTCTGTGGTTCTC
AGCATCAGAAGGCAGCGCAGCCCCTCTCCTCTTCAGGCTACAGGGTTGTCACCTGCTGAGTCCTC
AGGTTGTTTGGCCTCTCTGGTCCATCTTGGGCATTAGGTTCTCCAGCAGAGCTCTGGCCAGCTGC
CTCTTCTTTAACTGGGAACACAGGCTCTCACAAGATCAGAACCCCCACTCACCCCCAAGATCTTA
TCTAGCAAGCCTGTAGTATTCAGTTTCTGTTGTAGGAAGAGAGCGAGGCATCCCTGAATTCCACG
CATCTGCTGGAAACGAGCCGTGTCAGATCGCACATCCCTGCGCCCCCATGCCCCTCTGAGTCACA
CAGGACAGAGGAGGCAGAGCTTCTGCCCACTGTTATCTTCACTTTCTTTGTCCAGTCTTTTGTTT
TTAATAAGCAGTGACCCTCCCTACTCTTCTTTTTAATGATTTTTGTAGTTGATTTGTCTGAACTG
TGGCTACTGTGCATTCCTTGAATAATCACTTGTAAAAATTGTCAGTGCTTGAAGCTGTTTCCTTT
ACTCACATTGAAGGGACTTCGTTGGTTTTTTGGAGTCTTGGTTGTGACTCCAAGAGCAGAGTGAG
GAAGACCCCCAAGCATAGACTCGGGTACTGTGATGATGGCTGCAGTCCAGTTTTATGATTCTGCT
TTTATGTGTCCCTTGATAACAGTGACTTAACAATATACATTCCTCATAAATAAAAAAAAAACAAG
AATCTGAATTCTTAGAAA
NM_001193495.2
SEQ ID NO: 4
CCTGGCCAGCATGGTGAAACCCCATCTCTACTAAAAATACAAAAATTAGCTGGGTGTGGTGGCGT
GCGCCTGTAATCCCAGCTACTCCGGAGGCTGAGGCAGGAGAATCACTTGAACCCGGGAGGCGGAG
ATTGCAGTGAGCTGAGATCACACTGCACTCCAGCCTGATTGCAGTGAGCCGAGATCATGCCACTG
CACTCCAGCTTGGCAACAGAGCGAGACTCCGTCTCACAAGAAAAAAAATAACCGGGTATTCCCTC
AGCGGATACTACACCCATCCATTTCAAGGCTATGAGCACAGACAGCTCAGGTACCAGCAGCCTGG
GCCAGGATCTTCCCCCAGTAGTTTCCTGCTTAAGCAAATAGAATTTCTCAAGGGGCAGCTCCCAG
AAGCACCGGTGATTGGAAAGCAGACACCGTCACTGCCACCTTCCCTCCCAGGACTCCGGCCAAGG
TTTCCAGTACTACTTGCCTCCAGTACCAGAGGCAGGCAAGTGGACATCAGGGGTGTCCCCAGGGG
CGTGCATCTCAGAAGTCAGGGGCTCCAGAGAGGGTTCCAGCATCCTTCACCACGTGGCAGGAGTC
TGCCACAGAGAGGTGTTGATTGCCTTTCCTCACATTTCCAGGAACTGAGTATCTACCAAGATCAG
GAACAAAGGATCTTAAAGTTCCTGGAAGAGCTTGGGGAAGGGAAGGCCACCACAGCACATGATCT
GTCTGGGAAACTTGGGACTCCGAAGAAAGAAATCAATCGAGTTTTATACTCCCTGGCAAAGAAGG
GCAAGCTACAGAAAGAGGCAGGAACACCCCCTTTGTGGAAAATCGCGGTCTCCACTCAGGCTTGG
AACCAGCACAGCGGAGTGGTAAGACCAGACGGTCATAGCCAAGGAGCCCCAAACTCAGACCCGAG
TTTGGAACCGGAAGACAGAAACTCCACATCTGTCTCAGAAGATCTTCTTGAGCCTTTTATTGCAG
TCTCAGCTCAGGCTTGGAACCAGCACAGCGGAGTGGTAAGACCAGACAGTCATAGCCAAGGATCC
CCAAACTCAGACCCAGGTTTGGAACCTGAAGACAGCAACTCCACATCTGCCTTGGAAGATCCTCT
TGAGTTTTTAGACATGGCCGAGATCAAGGAGAAAATCTGCGACTATCTCTTCAATGTGTCTGACT
CCTCTGCCCTGAATTTGGCTAAAAATATTGGCCTTACCAAGGCCCGAGATATAAATGCTGTGCTA
ATTGACATGGAAAGGCAGGGGGATGTCTATAGACAAGGGACAACCCCTCCCATATGGCATTTGAC
AGACAAGAAGCGAGAGAGGATGCAAATCAAGAGAAATACGAACAGTGTTCCTGAAACCGCTCCAG
CTGCAATCCCTGAGACCAAAAGAAACGCAGAGTTCCTCACCTGTAATATACCCACATCAAATGCC
TCAAATAACATGGTAACCACAGAAAAAGTGGAGAATGGGCAGGAACCTGTCATAAAGTTAGAAAA
CAGGCAAGAGGCCAGACCAGAACCAGCAAGACTGAAACCACCTGTTCATTACAATGGCCCCTCAA
AAGCAGGGTATGTTGACTTTGAAAATGGCCAGTGGGCCACAGATGACATCCCAGATGACTTGAAT
AGTATCCGCGCAGCACCAGGTGAGTTTCGAGCCATCATGGAGATGCCCTCCTTCTACAGTCATGG
CTTGCCACGGTGTTCACCCTACAAGAAACTGACAGAGTGCCAGCTGAAGAACCCCATCAGCGGGC
TGTTAGAATATGCCCAGTTCGCTAGTCAAACCTGTGAGTTCAACATGATAGAGCAGAGTGGACCA
CCCCATGAACCTCGATTTAAATTCCAGGTTGTCATCAATGGCCGAGAGTTTCCCCCAGCTGAAGC
TGGAAGCAAGAAAGTGGCCAAGCAGGATGCAGCTATGAAAGCCATGACAATTCTGCTAGAGGAAG
CCAAAGCCAAGGACAGTGGAAAATCAGAAGAATCATCCCACTATTCCACAGAGAAAGAATCAGAG
AAGACTGCAGAGTCCCAGACCCCCACCCCTTCAGCCACATCCTTCTTTTCTGGGAAGAGCCCCGT
CACCACACTGCTTGAGTGTATGCACAAATTGGGGAACTCCTGCGAATTCCGTCTCCTGTCCAAAG
AAGGCCCTGCCCATGAACCCAAGTTCCAATACTGTGTTGCAGTGGGAGCCCAAACTTTCCCCAGT
GTGAGTGCTCCCAGCAAGAAAGTGGCAAAGCAGATGGCCGCAGAGGAAGCCATGAAGGCCCTGCA
TGGGGAGGCGACCAACTCCATGGCTTCTGATAACCAGCCTGAAGGTATGATCTCAGAGTCACTTG
ATAACTTGGAATCCATGATGCCCAACAAGGTCAGGAAGATTGGCGAGCTCGTGAGATACCTGAAC
ACCAACCCTGTGGGTGGCCTTTTGGAGTACGCCCGCTCCCATGGCTTTGCTGCTGAATTCAAGTT
GGTCGACCAGTCCGGACCTCCTCACGAGCCCAAGTTCGTTTACCAAGCAAAAGTTGGGGGTCGCT
GGTTCCCAGCCGTCTGCGCACACAGCAAGAAGCAAGGCAAGCAGGAAGCAGCAGATGCGGCTCTC
CGTGTCTTGATTGGGGAGAACGAGAAGGCAGAACGCATGGGTTTCACAGAGGTAACCCCAGTGAC
AGGGGCCAGTCTCAGAAGAACTATGCTCCTCCTCTCAAGGTCCCCAGAAGCACAGCCAAAGACAC
TCCCTCTCACTGGCAGCACCTTCCATGACCAGATAGCCATGCTGAGCCACCGGTGCTTCAACACT
CTGACTAACAGCTTCCAGCCCTCCTTGCTCGGCCGCAAGATTCTGGCCGCCATCATTATGAAAAA
AGACTCTGAGGACATGGGTGTCGTCGTCAGCTTGGGAACAGGGAATCGCTGTGTGAAAGGAGATT
CTCTCAGCCTAAAAGGAGAAACTGTCAATGACTGCCATGCAGAAATAATCTCCCGGAGAGGCTTC
ATCAGGTTTCTCTACAGTGAGTTAATGAAATACAACTCCCAGACTGCGAAGGATAGTATATTTGA
ACCTGCTAAGGGAGGAGAAAAGCTCCAAATAAAAAAGACTGTGTCATTCCATCTGTATATCAGCA
CTGCTCCGTGTGGAGATGGCGCCCTCTTTGACAAGTCCTGCAGCGACCGTGCTATGGAAAGCACA
GAATCCCGCCACTACCCTGTCTTCGAGAATCCCAAACAAGGAAAGCTCCGCACCAAGGTGGAGAA
CGGAGAAGGCACAATCCCTGTGGAATCCAGTGACATTGTGCCTACGTGGGATGGCATTCGGCTCG
GGGAGAGACTCCGTACCATGTCCTGTAGTGACAAAATCCTACGCTGGAACGTGCTGGGCCTGCAA
GGGGCACTGTTGACCCACTTCCTGCAGCCCATTTATCTCAAATCTGTCACATTGGGTTACCTTTT
CAGCCAAGGGCATCTGACCCGTGCTATTTGCTGTCGTGTGACAAGAGATGGGAGTGCATTTGAGG
ATGGACTACGACATCCCTTTATTGTCAACCACCCCAAGGTTGGCAGAGTCAGCATATATGATTCC
AAAAGGCAATCCGGGAAGACTAAGGAGACAAGCGTCAACTGGTGTCTGGCTGATGGCTATGACCT
GGAGATCCTGGACGGTACCAGAGGCACTGTGGATGGGCCACGGAATGAATTGTCCCGGGTCTCCA
AAAAGAACATTTTTCTTCTATTTAAGAAGCTCTGCTCCTTCCGTTACCGCAGGGATCTACTGAGA
CTCTCCTATGGTGAGGCCAAGAAAGCTGCCCGTGACTACGAGACGGCCAAGAACTACTTCAAAAA
AGGCCTGAAGGATATGGGCTATGGGAACTGGATTAGCAAACCCCAGGAGGAAAAGAACTTTTATC
TCTGCCCAGTATAGTATGCTCCAGTGACAGATGGATTAGGGTGTGTCATACTAGGGTGTGAGAGA
GGTAGGTCGTAGCATTCCTCATCACATGGTCAGGGGATTTTTTTTTCTCCTTTTTTTTTCTTTTT
AAGCCATAATTGGTGATACTGAAAACTTTGGGTTCCCATTTATCCTGCTTTCTTTGGGATTGCTA
GGCAAGGTCTGGCCAGGCCCCCCTTTTTTCCCCCAAGTGAAGAGGCAGAAACCTAAGAAGTTATC
TTTTCTTTCTACCCAAAGCATACATAGTCACTGAGCACCTGCGGTCCATTTCCTCTTAAAAGTTT
TGTTTTGATTTGTTTCCATTTCCTTTCCCTTTGTGTTTGCTACACTGACCTCTTGCGGTCTTGAT
TAGGTTTCAGTCAACTCTGGATCATGTCAGGGACTGATAATTTCATTTGTGGATTACGCAGACCC
CTCTACTTCCCCTCTTTCCCTTCTGAGATTCTTTCCTTGTGATCTGAATGTCTCCTTTTCCCCCT
CAGAGGGCAAAGAGGTGAACATAAAGGATTTGGTGAAACATTTGTAAGGGTAGGAGTTGAAAACT
GCAGTTCCCAGTGCCACGGAAGTGTGATTGGAGCCTGCAGATAATGCCCAGCCATCCTCCCATCC
TGCACTTTAGCCAGCTGCAGGGCGGGCAAGGCAAGGAAAGCTGCTTCCCTGGAAGTGTATCACTT
TCTCCGGCAGCTGGGAAGTCTAGAACCAGCCAGACTGGGTTAAGGGAGCTGCTCAAGCAATAGCA
GAGGTTTCACCCGGCAGGATGACACAGACCACTTCCCAGGGAGCACGGGCATGCCTTGGAATATT
GCCAAGCTTCCAGCTGCCTCTTCTCCTAAAGCATTCCTAGGAATATTTTCCCCGCCAATGCTGGG
CGTACACCCTAGCCAACGGGACAAATCCTAGAGGGTATAAAATCATCTCTGCTCAGATAATCATG
ACTTAGCAAGAATAAGGGCAAAAAATCCTGTTGGCTTAACGTCACTGTTCCACCCGGTGTAATAT
CTCTCATGACAGTGACACCAAGGGAAGTTGACTAAGTCACATGTAAATTAGGAGTGTTTTAAAGA
ATGCCATAGATGTTGATTCTTAACTGCTACAGATAACCTGTAATTGAGCAGATTTAAAATTCAGG
CATACTTTTCCATTTATCCAAGTGCTTTCATTTTTCCAGATGGCTTCAGAAGTAGGCTCGTGGGC
AGGGCGCAGACCTGATCTTTATAGGGTTGACATAGAAAGCAGTAGTTGTGGGTGAAAGGGCAGGT
TGTCTTCAAACTCTGTGAGGTAGAATCCTTTGTCTATACCTCCATGAACATTGACTCGTGTGTTC
AGAGCCTTTGGCCTCTCTGTGGAGTCTGGCTCTCTGGCTCCTGTGCATTCTTTGAATAGTCACTC
GTAAAAACTGTCAGTGCTTGAAACTGTTTCCTTTACTCATGTTGAAGGGACTTTGTTGGCTTTTA
GAGTGTTGGTCATGACTCCAAGAGCAGAGCAGGGAAGAGCCCAAGCATAGACTTGGTGCCGTGGT
GATGGCTGCAGTCCAGTTTTGTGATGCTGCTTTTACGTGTCCCTCGATAACAGTCAGCTAGACAC
ACTCAGGAGGACTACTGAGGCTCTGCGACCTTCAGGAGCTGAGCCTGCCTCTCTCCTTTAGATGA
CAGACCTTCATCTGGGAACGTGCTGAGCCAGCACCCTCAGATGATTTCCCTCCAAACTGCTGACT
AGGTCATCCTCTGTCTGGTAGAGACATTCACATCTTTGCTTTTATTCTATGCTCTCTGTACTTTT
GACCAAAAATTGACCAAAGTAAGAAAATGCAAGTTCTAAAAATAGACTAAGGATGCCTTTGCAGA
ACACCAAAGCATCCCAAGGAACTGGTAGGGAAGTGGCGCCTGTCTCCTGGAGTGGAAGAGGCCTG
CTCCCTGGCTCTGGGTCTGCTGGGGGCACAGTAAATCAGTCTTGGCACCCACATCCAGGGCAGAG
AGGTCTGTGGTTCTCAGCATCAGAAGGCAGCGCAGCCCCTCTCCTCTTCAGGCTACAGGGTTGTC
ACCTGCTGAGTCCTCAGGTTGTTTGGCCTCTCTGGTCCATCTTGGGCATTAGGTTCTCCAGCAGA
GCTCTGGCCAGCTGCCTCTTCTTTAACTGGGAACACAGGCTCTCACAAGATCAGAACCCCCACTC
ACCCCCAAGATCTTATCTAGCAAGCCTGTAGTATTCAGTTTCTGTTGTAGGAAGAGAGCGAGGCA
TCCCTGAATTCCACGCATCTGCTGGAAACGAGCCGTGTCAGATCGCACATCCCTGCGCCCCCATG
CCCCTCTGAGTCACACAGGACAGAGGAGGCAGAGCTTCTGCCCACTGTTATCTTCACTTTCTTTG
TCCAGTCTTTTGTTTTTAATAAGCAGTGACCCTCCCTACTCTTCTTTTTAATGATTTTTGTAGTT
GATTTGTCTGAACTGTGGCTACTGTGCATTCCTTGAATAATCACTTGTAAAAATTGTCAGTGCTT
GAAGCTGTTTCCTTTACTCACATTGAAGGGACTTCGTTGGTTTTTTGGAGTCTTGGTTGTGACTC
CAAGAGCAGAGTGAGGAAGACCCCCAAGCATAGACTCGGGTACTGTGATGATGGCTGCAGTCCAG
TTTTATGATTCTGCTTTTATGTGTCCCTTGATAACAGTGACTTAACAATATACATTCCTCATAAA
TAAAAAAAAAACAAGAATCTGAATTCTTAGAAA
NM_001365047.1
SEQ ID NO: 5
GAACCGGAGCCATCTTGGGCCCGGCGCGCAGACCCGCGGAGTTTCCCGTGCCGACGCCCCGGGGC
CACTTCCAGTGCGGAGTAGCGGAGGCGTGGGGGCCTCGAGGGGCTGGCGCGGCCCAGCGGTCGGG
CCAGGGTCGTGCCGCCGGCGGGTCGGGCCGGGCAATGCCTCGCGGGCGCAATGAATCCGCGGCAG
GGGTATTCCCTCAGCGGATACTACACCCATCCATTTCAAGGCTATGAGCACAGACAGCTCAGGTA
CCAGCAGCCTGGGCCAGGATCTTCCCCCAGTAGTTTCCTGCTTAAGCAAATAGAATTTCTCAAGG
GGCAGCTCCCAGAAGCACCGGTGATTGGAAAGCAGACACCGTCACTGCCACCTTCCCTCCCAGGA
CTCCGGCCAAGGTTTCCAGTACTACTTGCCTCCAGTACCAGAGGCAGGAACTGAGTATCTACCAA
GATCAGGAACAAAGGATCTTAAAGTTCCTGGAAGAGCTTGGGGAAGGGAAGGCCACCACAGCACA
TGATCTGTCTGGGAAACTTGGGACTCCGAAGAAAGAAATCAATCGAGTTTTATACTCCCTGGCAA
AGAAGGGCAAGCTACAGAAAGAGGCAGGAACACCCCCTTTGTGGAAAATCGCGGTCTCCACTCAG
GCTTGGAACCAGCACAGCGGAGTGGTAAGACCAGACGGTCATAGCCAAGGAGCCCCAAACTCAGA
CCCGAGTTTGGAACCGGAAGACAGAAACTCCACATCTGTCTCAGAAGATCTTCTTGAGCCTTTTA
TTGCAGTCTCAGCTCAGGCTTGGAACCAGCACAGCGGAGTGGTAAGACCAGACAGTCATAGCCAA
GGATCCCCAAACTCAGACCCAGGTTTGGAACCTGAAGACAGCAACTCCACATCTGCCTTGGAAGA
TCCTCTTGAGTTTTTAGACATGGCCGAGATCAAGGAGAAAATCTGCGACTATCTCTTCAATGTGT
CTGACTCCTCTGCCCTGAATTTGGCTAAAAATATTGGCCTTACCAAGGCCCGAGATATAAATGCT
GTGCTAATTGACATGGAAAGGCAGGGGGATGTCTATAGACAAGGGACAACCCCTCCCATATGGCA
TTTGACAGACAAGAAGCGAGAGAGGATGCAAATCAAGAGAAATACGAACAGTGTTCCTGAAACCG
CTCCAGCTGCAATCCCTGAGACCAAAAGAAACGCAGAGTTCCTCACCTGTAATATACCCACATCA
AATGCCTCAAATAACATGGTAACCACAGAAAAAGTGGAGAATGGGCAGGAACCTGTCATAAAGTT
AGAAAACAGGCAAGAGGCCAGACCAGAACCAGCAAGACTGAAACCACCTGTTCATTACAATGGCC
CCTCAAAAGCAGGGTATGTTGACTTTGAAAATGGCCAGTGGGCCACAGATGACATCCCAGATGAC
TTGAATAGTATCCGCGCAGCACCAGGTGAGTTTCGAGCCATCATGGAGATGCCCTCCTTCTACAG
TCATGGCTTGCCACGGTGTTCACCCTACAAGAAACTGACAGAGTGCCAGCTGAAGAACCCCATCA
GCGGGCTGTTAGAATATGCCCAGTTCGCTAGTCAAACCTGTGAGTTCAACATGATAGAGCAGAGT
GGACCACCCCATGAACCTCGATTTAAATTCCAGGTTGTCATCAATGGCCGAGAGTTTCCCCCAGC
TGAAGCTGGAAGCAAGAAAGTGGCCAAGCAGGATGCAGCTATGAAAGCCATGACAATTCTGCTAG
AGGAAGCCAAAGCCAAGGACAGTGGAAAATCAGAAGAATCATCCCACTATTCCACAGAGAAAGAA
TCAGAGAAGACTGCAGAGTCCCAGACCCCCACCCCTTCAGCCACATCCTTCTTTTCTGGGAAGAG
CCCCGTCACCACACTGCTTGAGTGTATGCACAAATTGGGGAACTCCTGCGAATTCCGTCTCCTGT
CCAAAGAAGGCCCTGCCCATGAACCCAAGTTCCAATACTGTGTTGCAGTGGGAGCCCAAACTTTC
CCCAGTGTGAGTGCTCCCAGCAAGAAAGTGGCAAAGCAGATGGCCGCAGAGGAAGCCATGAAGGC
CCTGCATGGGGAGGCGACCAACTCCATGGCTTCTGATAACCAGCCTGAAGGTATGATCTCAGAGT
CACTTGATAACTTGGAATCCATGATGCCCAACAAGGTCAGGAAGATTGGCGAGCTCGTGAGATAC
CTGAACACCAACCCTGTGGGTGGCCTTTTGGAGTACGCCCGCTCCCATGGCTTTGCTGCTGAATT
CAAGTTGGTCGACCAGTCCGGACCTCCTCACGAGCCCAAGTTCGTTTACCAAGCAAAAGTTGGGG
GTCGCTGGTTCCCAGCCGTCTGCGCACACAGCAAGAAGCAAGGCAAGCAGGAAGCAGCAGATGCG
GCTCTCCGTGTCTTGATTGGGGAGAACGAGAAGGCAGAACGCATGGGTTTCACAGAGGTAACCCC
AGTGACAGGGGCCAGTCTCAGAAGAACTATGCTCCTCCTCTCAAGGTCCCCAGAAGCACAGCCAA
AGACACTCCCTCTCACTGGCAGCACCTTCCATGACCAGATAGCCATGCTGAGCCACCGGTGCTTC
AACACTCTGACTAACAGCTTCCAGCCCTCCTTGCTCGGCCGCAAGATTCTGGCCGCCATCATTAT
GAAAAAAGACTCTGAGGACATGGGTGTCGTCGTCAGCTTGGGAACAGGGAATCGCTGTGTGAAAG
GAGATTCTCTCAGCCTAAAAGGAGAAACTGTCAATGACTGCCATGCAGAAATAATCTCCCGGAGA
GGCTTCATCAGGTTTCTCTACAGTGAGTTAATGAAATACAACTCCCAGACTGCGAAGGATAGTAT
ATTTGAACCTGCTAAGGGAGGAGAAAAGCTCCAAATAAAAAAGACTGTGTCATTCCATCTGTATA
TCAGCACTGCTCCGTGTGGAGATGGCGCCCTCTTTGACAAGTCCTGCAGCGACCGTGCTATGGAA
AGCACAGAATCCCGCCACTACCCTGTCTTCGAGAATCCCAAACAAGGAAAGCTCCGCACCAAGGT
GGAGAACGGAGAAGGCACAATCCCTGTGGAATCCAGTGACATTGTGCCTACGTGGGATGGCATTC
GGCTCGGGGAGAGACTCCGTACCATGTCCTGTAGTGACAAAATCCTACGCTGGAACGTGCTGGGC
CTGCAAGGGGCACTGTTGACCCACTTCCTGCAGCCCATTTATCTCAAATCTGTCACATTGGGTTA
CCTTTTCAGCCAAGGGCATCTGACCCGTGCTATTTGCTGTCGTGTGACAAGAGATGGGAGTGCAT
TTGAGGATGGACTACGACATCCCTTTATTGTCAACCACCCCAAGGTTGGCAGAGTCAGCATATAT
GATTCCAAAAGGCAATCCGGGAAGACTAAGGAGACAAGCGTCAACTGGTGTCTGGCTGATGGCTA
TGACCTGGAGATCCTGGACGGTACCAGAGGCACTGTGGATGGGCCACGGAATGAATTGTCCCGGG
TCTCCAAAAAGAACATTTTTCTTCTATTTAAGAAGCTCTGCTCCTTCCGTTACCGCAGGGATCTA
CTGAGACTCTCCTATGGTGAGGCCAAGAAAGCTGCCCGTGACTACGAGACGGCCAAGAACTACTT
CAAAAAAGGCCTGAAGGATATGGGCTATGGGAACTGGATTAGCAAACCCCAGGAGGAAAAGAACT
TTTATCTCTGCCCAGTATAGTATGCTCCAGTGACAGATGGATTAGGGTGTGTCATACTAGGGTGT
GAGAGAGGTAGGTCGTAGCATTCCTCATCACATGGTCAGGGGATTTTTTTTTCTCCTTTTTTTTT
CTTTTTAAGCCATAATTGGTGATACTGAAAACTTTGGGTTCCCATTTATCCTGCTTTCTTTGGGA
TTGCTAGGCAAGGTCTGGCCAGGCCCCCCTTTTTTCCCCCAAGTGAAGAGGCAGAAACCTAAGAA
GTTATCTTTTCTTTCTACCCAAAGCATACATAGTCACTGAGCACCTGCGGTCCATTTCCTCTTAA
AAGTTTTGTTTTGATTTGTTTCCATTTCCTTTCCCTTTGTGTTTGCTACACTGACCTCTTGCGGT
CTTGATTAGGTTTCAGTCAACTCTGGATCATGTCAGGGACTGATAATTTCATTTGTGGATTACGC
AGACCCCTCTACTTCCCCTCTTTCCCTTCTGAGATTCTTTCCTTGTGATCTGAATGTCTCCTTTT
CCCCCTCAGAGGGCAAAGAGGTGAACATAAAGGATTTGGTGAAACATTTGTAAGGGTAGGAGTTG
AAAACTGCAGTTCCCAGTGCCACGGAAGTGTGATTGGAGCCTGCAGATAATGCCCAGCCATCCTC
CCATCCTGCACTTTAGCCAGCTGCAGGGCGGGCAAGGCAAGGAAAGCTGCTTCCCTGGAAGTGTA
TCACTTTCTCCGGCAGCTGGGAAGTCTAGAACCAGCCAGACTGGGTTAAGGGAGCTGCTCAAGCA
ATAGCAGAGGTTTCACCCGGCAGGATGACACAGACCACTTCCCAGGGAGCACGGGCATGCCTTGG
AATATTGCCAAGCTTCCAGCTGCCTCTTCTCCTAAAGCATTCCTAGGAATATTTTCCCCGCCAAT
GCTGGGCGTACACCCTAGCCAACGGGACAAATCCTAGAGGGTATAAAATCATCTCTGCTCAGATA
ATCATGACTTAGCAAGAATAAGGGCAAAAAATCCTGTTGGCTTAACGTCACTGTTCCACCCGGTG
TAATATCTCTCATGACAGTGACACCAAGGGAAGTTGACTAAGTCACATGTAAATTAGGAGTGTTT
TAAAGAATGCCATAGATGTTGATTCTTAACTGCTACAGATAACCTGTAATTGAGCAGATTTAAAA
TTCAGGCATACTTTTCCATTTATCCAAGTGCTTTCATTTTTCCAGATGGCTTCAGAAGTAGGCTC
GTGGGCAGGGCGCAGACCTGATCTTTATAGGGTTGACATAGAAAGCAGTAGTTGTGGGTGAAAGG
GCAGGTTGTCTTCAAACTCTGTGAGGTAGAATCCTTTGTCTATACCTCCATGAACATTGACTCGT
GTGTTCAGAGCCTTTGGCCTCTCTGTGGAGTCTGGCTCTCTGGCTCCTGTGCATTCTTTGAATAG
TCACTCGTAAAAACTGTCAGTGCTTGAAACTGTTTCCTTTACTCATGTTGAAGGGACTTTGTTGG
CTTTTAGAGTGTTGGTCATGACTCCAAGAGCAGAGCAGGGAAGAGCCCAAGCATAGACTTGGTGC
CGTGGTGATGGCTGCAGTCCAGTTTTGTGATGCTGCTTTTACGTGTCCCTCGATAACAGTCAGCT
AGACACACTCAGGAGGACTACTGAGGCTCTGCGACCTTCAGGAGCTGAGCCTGCCTCTCTCCTTT
AGATGACAGACCTTCATCTGGGAACGTGCTGAGCCAGCACCCTCAGATGATTTCCCTCCAAACTG
CTGACTAGGTCATCCTCTGTCTGGTAGAGACATTCACATCTTTGCTTTTATTCTATGCTCTCTGT
ACTTTTGACCAAAAATTGACCAAAGTAAGAAAATGCAAGTTCTAAAAATAGACTAAGGATGCCTT
TGCAGAACACCAAAGCATCCCAAGGAACTGGTAGGGAAGTGGCGCCTGTCTCCTGGAGTGGAAGA
GGCCTGCTCCCTGGCTCTGGGTCTGCTGGGGGCACAGTAAATCAGTCTTGGCACCCACATCCAGG
GCAGAGAGGTCTGTGGTTCTCAGCATCAGAAGGCAGCGCAGCCCCTCTCCTCTTCAGGCTACAGG
GTTGTCACCTGCTGAGTCCTCAGGTTGTTTGGCCTCTCTGGTCCATCTTGGGCATTAGGTTCTCC
AGCAGAGCTCTGGCCAGCTGCCTCTTCTTTAACTGGGAACACAGGCTCTCACAAGATCAGAACCC
CCACTCACCCCCAAGATCTTATCTAGCAAGCCTGTAGTATTCAGTTTCTGTTGTAGGAAGAGAGC
GAGGCATCCCTGAATTCCACGCATCTGCTGGAAACGAGCCGTGTCAGATCGCACATCCCTGCGCC
CCCATGCCCCTCTGAGTCACACAGGACAGAGGAGGCAGAGCTTCTGCCCACTGTTATCTTCACTT
TCTTTGTCCAGTCTTTTGTTTTTAATAAGCAGTGACCCTCCCTACTCTTCTTTTTAATGATTTTT
GTAGTTGATTTGTCTGAACTGTGGCTACTGTGCATTCCTTGAATAATCACTTGTAAAAATTGTCA
GTGCTTGAAGCTGTTTCCTTTACTCACATTGAAGGGACTTCGTTGGTTTTTTGGAGTCTTGGTTG
TGACTCCAAGAGCAGAGTGAGGAAGACCCCCAAGCATAGACTCGGGTACTGTGATGATGGCTGCA
GTCCAGTTTTATGATTCTGCTTTTATGTGTCCCTTGATAACAGTGACTTAACAATATACATTCCT
CATAAATAAAAAAAAAACAAGAATCTGAATTCTTAGAAA
NM_001365048.1
SEQ ID NO: 6
GGCGCGGAGAGAGGGAGGACCGGGCGTCATGCTGTTTCTGGCCTGAGGGGTATTCCCTCAGCGGA
TACTACACCCATCCATTTCAAGGCTATGAGCACAGACAGCTCAGGTACCAGCAGCCTGGGCCAGG
ATCTTCCCCCAGTAGTTTCCTGCTTAAGCAAATAGAATTTCTCAAGGGGCAGCTCCCAGAAGCAC
CGGTGATTGGAAAGCAGACACCGTCACTGCCACCTTCCCTCCCAGGACTCCGGCCAAGGTTTCCA
GTACTACTTGCCTCCAGTACCAGAGGCAGGCAAGTGGACATCAGGGGTGTCCCCAGGGGCGTGCA
TCTCAGAAGTCAGGGGCTCCAGAGAGGGTTCCAGCATCCTTCACCACGTGGCAGGAGTCTGCCAC
AGAGAGGTGTTGATTGCCTTTCCTCACATTTCCAGGAACTGAGTATCTACCAAGATCAGGAACAA
AGGATCTTAAAGTTCCTGGAAGAGCTTGGGGAAGGGAAGGCCACCACAGCACATGATCTGTCTGG
GAAACTTGGGACTCCGAAGAAAGAAATCAATCGAGTTTTATACTCCCTGGCAAAGAAGGGCAAGC
TACAGAAAGAGGCAGGAACACCCCCTTTGTGGAAAATCGCGGTCTCCACTCAGGCTTGGAACCAG
CACAGCGGAGTGGTAAGACCAGACGGTCATAGCCAAGGAGCCCCAAACTCAGACCCGAGTTTGGA
ACCGGAAGACAGAAACTCCACATCTGTCTCAGAAGATCTTCTTGAGCCTTTTATTGCAGTCTCAG
CTCAGGCTTGGAACCAGCACAGCGGAGTGGTAAGACCAGACAGTCATAGCCAAGGATCCCCAAAC
TCAGACCCAGGTTTGGAACCTGAAGACAGCAACTCCACATCTGCCTTGGAAGATCCTCTTGAGTT
TTTAGACATGGCCGAGATCAAGGAGAAAATCTGCGACTATCTCTTCAATGTGTCTGACTCCTCTG
CCCTGAATTTGGCTAAAAATATTGGCCTTACCAAGGCCCGAGATATAAATGCTGTGCTAATTGAC
ATGGAAAGGCAGGGGGATGTCTATAGACAAGGGACAACCCCTCCCATATGGCATTTGACAGACAA
GAAGCGAGAGAGGATGCAAATCAAGAGAAATACGAACAGTGTTCCTGAAACCGCTCCAGCTGCAA
TCCCTGAGACCAAAAGAAACGCAGAGTTCCTCACCTGTAATATACCCACATCAAATGCCTCAAAT
AACATGGTAACCACAGAAAAAGTGGAGAATGGGCAGGAACCTGTCATAAAGTTAGAAAACAGGCA
AGAGGCCAGACCAGAACCAGCAAGACTGAAACCACCTGTTCATTACAATGGCCCCTCAAAAGCAG
GGTATGTTGACTTTGAAAATGGCCAGTGGGCCACAGATGACATCCCAGATGACTTGAATAGTATC
CGCGCAGCACCAGGTGAGTTTCGAGCCATCATGGAGATGCCCTCCTTCTACAGTCATGGCTTGCC
ACGGTGTTCACCCTACAAGAAACTGACAGAGTGCCAGCTGAAGAACCCCATCAGCGGGCTGTTAG
AATATGCCCAGTTCGCTAGTCAAACCTGTGAGTTCAACATGATAGAGCAGAGTGGACCACCCCAT
GAACCTCGATTTAAATTCCAGGTTGTCATCAATGGCCGAGAGTTTCCCCCAGCTGAAGCTGGAAG
CAAGAAAGTGGCCAAGCAGGATGCAGCTATGAAAGCCATGACAATTCTGCTAGAGGAAGCCAAAG
CCAAGGACAGTGGAAAATCAGAAGAATCATCCCACTATTCCACAGAGAAAGAATCAGAGAAGACT
GCAGAGTCCCAGACCCCCACCCCTTCAGCCACATCCTTCTTTTCTGGGAAGAGCCCCGTCACCAC
ACTGCTTGAGTGTATGCACAAATTGGGGAACTCCTGCGAATTCCGTCTCCTGTCCAAAGAAGGCC
CTGCCCATGAACCCAAGTTCCAATACTGTGTTGCAGTGGGAGCCCAAACTTTCCCCAGTGTGAGT
GCTCCCAGCAAGAAAGTGGCAAAGCAGATGGCCGCAGAGGAAGCCATGAAGGCCCTGCATGGGGA
GGCGACCAACTCCATGGCTTCTGATAACCAGCCTGAAGGTATGATCTCAGAGTCACTTGATAACT
TGGAATCCATGATGCCCAACAAGGTCAGGAAGATTGGCGAGCTCGTGAGATACCTGAACACCAAC
CCTGTGGGTGGCCTTTTGGAGTACGCCCGCTCCCATGGCTTTGCTGCTGAATTCAAGTTGGTCGA
CCAGTCCGGACCTCCTCACGAGCCCAAGTTCGTTTACCAAGCAAAAGTTGGGGGTCGCTGGTTCC
CAGCCGTCTGCGCACACAGCAAGAAGCAAGGCAAGCAGGAAGCAGCAGATGCGGCTCTCCGTGTC
TTGATTGGGGAGAACGAGAAGGCAGAACGCATGGGTTTCACAGAGGTAACCCCAGTGACAGGGGC
CAGTCTCAGAAGAACTATGCTCCTCCTCTCAAGGTCCCCAGAAGCACAGCCAAAGACACTCCCTC
TCACTGGCAGCACCTTCCATGACCAGATAGCCATGCTGAGCCACCGGTGCTTCAACACTCTGACT
AACAGCTTCCAGCCCTCCTTGCTCGGCCGCAAGATTCTGGCCGCCATCATTATGAAAAAAGACTC
TGAGGACATGGGTGTCGTCGTCAGCTTGGGAACAGGGAATCGCTGTGTGAAAGGAGATTCTCTCA
GCCTAAAAGGAGAAACTGTCAATGACTGCCATGCAGAAATAATCTCCCGGAGAGGCTTCATCAGG
TTTCTCTACAGTGAGTTAATGAAATACAACTCCCAGACTGCGAAGGATAGTATATTTGAACCTGC
TAAGGGAGGAGAAAAGCTCCAAATAAAAAAGACTGTGTCATTCCATCTGTATATCAGCACTGCTC
CGTGTGGAGATGGCGCCCTCTTTGACAAGTCCTGCAGCGACCGTGCTATGGAAAGCACAGAATCC
CGCCACTACCCTGTCTTCGAGAATCCCAAACAAGGAAAGCTCCGCACCAAGGTGGAGAACGGAGA
AGGCACAATCCCTGTGGAATCCAGTGACATTGTGCCTACGTGGGATGGCATTCGGCTCGGGGAGA
GACTCCGTACCATGTCCTGTAGTGACAAAATCCTACGCTGGAACGTGCTGGGCCTGCAAGGGGCA
CTGTTGACCCACTTCCTGCAGCCCATTTATCTCAAATCTGTCACATTGGGTTACCTTTTCAGCCA
AGGGCATCTGACCCGTGCTATTTGCTGTCGTGTGACAAGAGATGGGAGTGCATTTGAGGATGGAC
TACGACATCCCTTTATTGTCAACCACCCCAAGGTTGGCAGAGTCAGCATATATGATTCCAAAAGG
CAATCCGGGAAGACTAAGGAGACAAGCGTCAACTGGTGTCTGGCTGATGGCTATGACCTGGAGAT
CCTGGACGGTACCAGAGGCACTGTGGATGGGCCACGGAATGAATTGTCCCGGGTCTCCAAAAAGA
ACATTTTTCTTCTATTTAAGAAGCTCTGCTCCTTCCGTTACCGCAGGGATCTACTGAGACTCTCC
TATGGTGAGGCCAAGAAAGCTGCCCGTGACTACGAGACGGCCAAGAACTACTTCAAAAAAGGCCT
GAAGGATATGGGCTATGGGAACTGGATTAGCAAACCCCAGGAGGAAAAGAACTTTTATCTCTGCC
CAGTATAGTATGCTCCAGTGACAGATGGATTAGGGTGTGTCATACTAGGGTGTGAGAGAGGTAGG
TCGTAGCATTCCTCATCACATGGTCAGGGGATTTTTTTTTCTCCTTTTTTTTTCTTTTTAAGCCA
TAATTGGTGATACTGAAAACTTTGGGTTCCCATTTATCCTGCTTTCTTTGGGATTGCTAGGCAAG
GTCTGGCCAGGCCCCCCTTTTTTCCCCCAAGTGAAGAGGCAGAAACCTAAGAAGTTATCTTTTCT
TTCTACCCAAAGCATACATAGTCACTGAGCACCTGCGGTCCATTTCCTCTTAAAAGTTTTGTTTT
GATTTGTTTCCATTTCCTTTCCCTTTGTGTTTGCTACACTGACCTCTTGCGGTCTTGATTAGGTT
TCAGTCAACTCTGGATCATGTCAGGGACTGATAATTTCATTTGTGGATTACGCAGACCCCTCTAC
TTCCCCTCTTTCCCTTCTGAGATTCTTTCCTTGTGATCTGAATGTCTCCTTTTCCCCCTCAGAGG
GCAAAGAGGTGAACATAAAGGATTTGGTGAAACATTTGTAAGGGTAGGAGTTGAAAACTGCAGTT
CCCAGTGCCACGGAAGTGTGATTGGAGCCTGCAGATAATGCCCAGCCATCCTCCCATCCTGCACT
TTAGCCAGCTGCAGGGCGGGCAAGGCAAGGAAAGCTGCTTCCCTGGAAGTGTATCACTTTCTCCG
GCAGCTGGGAAGTCTAGAACCAGCCAGACTGGGTTAAGGGAGCTGCTCAAGCAATAGCAGAGGTT
TCACCCGGCAGGATGACACAGACCACTTCCCAGGGAGCACGGGCATGCCTTGGAATATTGCCAAG
CTTCCAGCTGCCTCTTCTCCTAAAGCATTCCTAGGAATATTTTCCCCGCCAATGCTGGGCGTACA
CCCTAGCCAACGGGACAAATCCTAGAGGGTATAAAATCATCTCTGCTCAGATAATCATGACTTAG
CAAGAATAAGGGCAAAAAATCCTGTTGGCTTAACGTCACTGTTCCACCCGGTGTAATATCTCTCA
TGACAGTGACACCAAGGGAAGTTGACTAAGTCACATGTAAATTAGGAGTGTTTTAAAGAATGCCA
TAGATGTTGATTCTTAACTGCTACAGATAACCTGTAATTGAGCAGATTTAAAATTCAGGCATACT
TTTCCATTTATCCAAGTGCTTTCATTTTTCCAGATGGCTTCAGAAGTAGGCTCGTGGGCAGGGCG
CAGACCTGATCTTTATAGGGTTGACATAGAAAGCAGTAGTTGTGGGTGAAAGGGCAGGTTGTCTT
CAAACTCTGTGAGGTAGAATCCTTTGTCTATACCTCCATGAACATTGACTCGTGTGTTCAGAGCC
TTTGGCCTCTCTGTGGAGTCTGGCTCTCTGGCTCCTGTGCATTCTTTGAATAGTCACTCGTAAAA
ACTGTCAGTGCTTGAAACTGTTTCCTTTACTCATGTTGAAGGGACTTTGTTGGCTTTTAGAGTGT
TGGTCATGACTCCAAGAGCAGAGCAGGGAAGAGCCCAAGCATAGACTTGGTGCCGTGGTGATGGC
TGCAGTCCAGTTTTGTGATGCTGCTTTTACGTGTCCCTCGATAACAGTCAGCTAGACACACTCAG
GAGGACTACTGAGGCTCTGCGACCTTCAGGAGCTGAGCCTGCCTCTCTCCTTTAGATGACAGACC
TTCATCTGGGAACGTGCTGAGCCAGCACCCTCAGATGATTTCCCTCCAAACTGCTGACTAGGTCA
TCCTCTGTCTGGTAGAGACATTCACATCTTTGCTTTTATTCTATGCTCTCTGTACTTTTGACCAA
AAATTGACCAAAGTAAGAAAATGCAAGTTCTAAAAATAGACTAAGGATGCCTTTGCAGAACACCA
AAGCATCCCAAGGAACTGGTAGGGAAGTGGCGCCTGTCTCCTGGAGTGGAAGAGGCCTGCTCCCT
GGCTCTGGGTCTGCTGGGGGCACAGTAAATCAGTCTTGGCACCCACATCCAGGGCAGAGAGGTCT
GTGGTTCTCAGCATCAGAAGGCAGCGCAGCCCCTCTCCTCTTCAGGCTACAGGGTTGTCACCTGC
TGAGTCCTCAGGTTGTTTGGCCTCTCTGGTCCATCTTGGGCATTAGGTTCTCCAGCAGAGCTCTG
GCCAGCTGCCTCTTCTTTAACTGGGAACACAGGCTCTCACAAGATCAGAACCCCCACTCACCCCC
AAGATCTTATCTAGCAAGCCTGTAGTATTCAGTTTCTGTTGTAGGAAGAGAGCGAGGCATCCCTG
AATTCCACGCATCTGCTGGAAACGAGCCGTGTCAGATCGCACATCCCTGCGCCCCCATGCCCCTC
TGAGTCACACAGGACAGAGGAGGCAGAGCTTCTGCCCACTGTTATCTTCACTTTCTTTGTCCAGT
CTTTTGTTTTTAATAAGCAGTGACCCTCCCTACTCTTCTTTTTAATGATTTTTGTAGTTGATTTG
TCTGAACTGTGGCTACTGTGCATTCCTTGAATAATCACTTGTAAAAATTGTCAGTGCTTGAAGCT
GTTTCCTTTACTCACATTGAAGGGACTTCGTTGGTTTTTTGGAGTCTTGGTTGTGACTCCAAGAG
CAGAGTGAGGAAGACCCCCAAGCATAGACTCGGGTACTGTGATGATGGCTGCAGTCCAGTTTTAT
GATTCTGCTTTTATGTGTCCCTTGATAACAGTGACTTAACAATATACATTCCTCATAAATAAAAA
AAAAACAAGAATCTGAATTCTTAGAAA
NM_001111.5
SEQ ID NO: 7
GAACCGGAGCCATCTTGGGCCCGGCGCGCAGACCCGCGGAGTTTCCCGTGCCGACGCCCCGGGGC
CACTTCCAGTGCGGAGTAGCGGAGGCGTGGGGGCCTCGAGGGGCTGGCGCGGCCCAGCGGTCGGG
CCAGGGTCGTGCCGCCGGCGGGTCGGGCCGGGCAATGCCTCGCGGGCGCAATGAATCCGCGGCAG
GGGTATTCCCTCAGCGGATACTACACCCATCCATTTCAAGGCTATGAGCACAGACAGCTCAGGTA
CCAGCAGCCTGGGCCAGGATCTTCCCCCAGTAGTTTCCTGCTTAAGCAAATAGAATTTCTCAAGG
GGCAGCTCCCAGAAGCACCGGTGATTGGAAAGCAGACACCGTCACTGCCACCTTCCCTCCCAGGA
CTCCGGCCAAGGTTTCCAGTACTACTTGCCTCCAGTACCAGAGGCAGGCAAGTGGACATCAGGGG
TGTCCCCAGGGGCGTGCATCTCAGAAGTCAGGGGCTCCAGAGAGGGTTCCAGCATCCTTCACCAC
GTGGCAGGAGTCTGCCACAGAGAGGTGTTGATTGCCTTTCCTCACATTTCCAGGAACTGAGTATC
TACCAAGATCAGGAACAAAGGATCTTAAAGTTCCTGGAAGAGCTTGGGGAAGGGAAGGCCACCAC
AGCACATGATCTGTCTGGGAAACTTGGGACTCCGAAGAAAGAAATCAATCGAGTTTTATACTCCC
TGGCAAAGAAGGGCAAGCTACAGAAAGAGGCAGGAACACCCCCTTTGTGGAAAATCGCGGTCTCC
ACTCAGGCTTGGAACCAGCACAGCGGAGTGGTAAGACCAGACGGTCATAGCCAAGGAGCCCCAAA
CTCAGACCCGAGTTTGGAACCGGAAGACAGAAACTCCACATCTGTCTCAGAAGATCTTCTTGAGC
CTTTTATTGCAGTCTCAGCTCAGGCTTGGAACCAGCACAGCGGAGTGGTAAGACCAGACAGTCAT
AGCCAAGGATCCCCAAACTCAGACCCAGGTTTGGAACCTGAAGACAGCAACTCCACATCTGCCTT
GGAAGATCCTCTTGAGTTTTTAGACATGGCCGAGATCAAGGAGAAAATCTGCGACTATCTCTTCA
ATGTGTCTGACTCCTCTGCCCTGAATTTGGCTAAAAATATTGGCCTTACCAAGGCCCGAGATATA
AATGCTGTGCTAATTGACATGGAAAGGCAGGGGGATGTCTATAGACAAGGGACAACCCCTCCCAT
ATGGCATTTGACAGACAAGAAGCGAGAGAGGATGCAAATCAAGAGAAATACGAACAGTGTTCCTG
AAACCGCTCCAGCTGCAATCCCTGAGACCAAAAGAAACGCAGAGTTCCTCACCTGTAATATACCC
ACATCAAATGCCTCAAATAACATGGTAACCACAGAAAAAGTGGAGAATGGGCAGGAACCTGTCAT
AAAGTTAGAAAACAGGCAAGAGGCCAGACCAGAACCAGCAAGACTGAAACCACCTGTTCATTACA
ATGGCCCCTCAAAAGCAGGGTATGTTGACTTTGAAAATGGCCAGTGGGCCACAGATGACATCCCA
GATGACTTGAATAGTATCCGCGCAGCACCAGGTGAGTTTCGAGCCATCATGGAGATGCCCTCCTT
CTACAGTCATGGCTTGCCACGGTGTTCACCCTACAAGAAACTGACAGAGTGCCAGCTGAAGAACC
CCATCAGCGGGCTGTTAGAATATGCCCAGTTCGCTAGTCAAACCTGTGAGTTCAACATGATAGAG
CAGAGTGGACCACCCCATGAACCTCGATTTAAATTCCAGGTTGTCATCAATGGCCGAGAGTTTCC
CCCAGCTGAAGCTGGAAGCAAGAAAGTGGCCAAGCAGGATGCAGCTATGAAAGCCATGACAATTC
TGCTAGAGGAAGCCAAAGCCAAGGACAGTGGAAAATCAGAAGAATCATCCCACTATTCCACAGAG
AAAGAATCAGAGAAGACTGCAGAGTCCCAGACCCCCACCCCTTCAGCCACATCCTTCTTTTCTGG
GAAGAGCCCCGTCACCACACTGCTTGAGTGTATGCACAAATTGGGGAACTCCTGCGAATTCCGTC
TCCTGTCCAAAGAAGGCCCTGCCCATGAACCCAAGTTCCAATACTGTGTTGCAGTGGGAGCCCAA
ACTTTCCCCAGTGTGAGTGCTCCCAGCAAGAAAGTGGCAAAGCAGATGGCCGCAGAGGAAGCCAT
GAAGGCCCTGCATGGGGAGGCGACCAACTCCATGGCTTCTGATAACCAGCCTGAAGGTATGATCT
CAGAGTCACTTGATAACTTGGAATCCATGATGCCCAACAAGGTCAGGAAGATTGGCGAGCTCGTG
AGATACCTGAACACCAACCCTGTGGGTGGCCTTTTGGAGTACGCCCGCTCCCATGGCTTTGCTGC
TGAATTCAAGTTGGTCGACCAGTCCGGACCTCCTCACGAGCCCAAGTTCGTTTACCAAGCAAAAG
TTGGGGGTCGCTGGTTCCCAGCCGTCTGCGCACACAGCAAGAAGCAAGGCAAGCAGGAAGCAGCA
GATGCGGCTCTCCGTGTCTTGATTGGGGAGAACGAGAAGGCAGAACGCATGGGTTTCACAGAGGT
AACCCCAGTGACAGGGGCCAGTCTCAGAAGAACTATGCTCCTCCTCTCAAGGTCCCCAGAAGCAC
AGCCAAAGACACTCCCTCTCACTGGCAGCACCTTCCATGACCAGATAGCCATGCTGAGCCACCGG
TGCTTCAACACTCTGACTAACAGCTTCCAGCCCTCCTTGCTCGGCCGCAAGATTCTGGCCGCCAT
CATTATGAAAAAAGACTCTGAGGACATGGGTGTCGTCGTCAGCTTGGGAACAGGGAATCGCTGTG
TGAAAGGAGATTCTCTCAGCCTAAAAGGAGAAACTGTCAATGACTGCCATGCAGAAATAATCTCC
CGGAGAGGCTTCATCAGGTTTCTCTACAGTGAGTTAATGAAATACAACTCCCAGACTGCGAAGGA
TAGTATATTTGAACCTGCTAAGGGAGGAGAAAAGCTCCAAATAAAAAAGACTGTGTCATTCCATC
TGTATATCAGCACTGCTCCGTGTGGAGATGGCGCCCTCTTTGACAAGTCCTGCAGCGACCGTGCT
ATGGAAAGCACAGAATCCCGCCACTACCCTGTCTTCGAGAATCCCAAACAAGGAAAGCTCCGCAC
CAAGGTGGAGAACGGAGAAGGCACAATCCCTGTGGAATCCAGTGACATTGTGCCTACGTGGGATG
GCATTCGGCTCGGGGAGAGACTCCGTACCATGTCCTGTAGTGACAAAATCCTACGCTGGAACGTG
CTGGGCCTGCAAGGGGCACTGTTGACCCACTTCCTGCAGCCCATTTATCTCAAATCTGTCACATT
GGGTTACCTTTTCAGCCAAGGGCATCTGACCCGTGCTATTTGCTGTCGTGTGACAAGAGATGGGA
GTGCATTTGAGGATGGACTACGACATCCCTTTATTGTCAACCACCCCAAGGTTGGCAGAGTCAGC
ATATATGATTCCAAAAGGCAATCCGGGAAGACTAAGGAGACAAGCGTCAACTGGTGTCTGGCTGA
TGGCTATGACCTGGAGATCCTGGACGGTACCAGAGGCACTGTGGATGGGCCACGGAATGAATTGT
CCCGGGTCTCCAAAAAGAACATTTTTCTTCTATTTAAGAAGCTCTGCTCCTTCCGTTACCGCAGG
GATCTACTGAGACTCTCCTATGGTGAGGCCAAGAAAGCTGCCCGTGACTACGAGACGGCCAAGAA
CTACTTCAAAAAAGGCCTGAAGGATATGGGCTATGGGAACTGGATTAGCAAACCCCAGGAGGAAA
AGAACTTTTATCTCTGCCCAGTATAGTATGCTCCAGTGACAGATGGATTAGGGTGTGTCATACTA
GGGTGTGAGAGAGGTAGGTCGTAGCATTCCTCATCACATGGTCAGGGGATTTTTTTTTCTCCTTT
TTTTTTCTTTTTAAGCCATAATTGGTGATACTGAAAACTTTGGGTTCCCATTTATCCTGCTTTCT
TTGGGATTGCTAGGCAAGGTCTGGCCAGGCCCCCCTTTTTTCCCCCAAGTGAAGAGGCAGAAACC
TAAGAAGTTATCTTTTCTTTCTACCCAAAGCATACATAGTCACTGAGCACCTGCGGTCCATTTCC
TCTTAAAAGTTTTGTTTTGATTTGTTTCCATTTCCTTTCCCTTTGTGTTTGCTACACTGACCTCT
TGCGGTCTTGATTAGGTTTCAGTCAACTCTGGATCATGTCAGGGACTGATAATTTCATTTGTGGA
TTACGCAGACCCCTCTACTTCCCCTCTTTCCCTTCTGAGATTCTTTCCTTGTGATCTGAATGTCT
CCTTTTCCCCCTCAGAGGGCAAAGAGGTGAACATAAAGGATTTGGTGAAACATTTGTAAGGGTAG
GAGTTGAAAACTGCAGTTCCCAGTGCCACGGAAGTGTGATTGGAGCCTGCAGATAATGCCCAGCC
ATCCTCCCATCCTGCACTTTAGCCAGCTGCAGGGCGGGCAAGGCAAGGAAAGCTGCTTCCCTGGA
AGTGTATCACTTTCTCCGGCAGCTGGGAAGTCTAGAACCAGCCAGACTGGGTTAAGGGAGCTGCT
CAAGCAATAGCAGAGGTTTCACCCGGCAGGATGACACAGACCACTTCCCAGGGAGCACGGGCATG
CCTTGGAATATTGCCAAGCTTCCAGCTGCCTCTTCTCCTAAAGCATTCCTAGGAATATTTTCCCC
GCCAATGCTGGGCGTACACCCTAGCCAACGGGACAAATCCTAGAGGGTATAAAATCATCTCTGCT
CAGATAATCATGACTTAGCAAGAATAAGGGCAAAAAATCCTGTTGGCTTAACGTCACTGTTCCAC
CCGGTGTAATATCTCTCATGACAGTGACACCAAGGGAAGTTGACTAAGTCACATGTAAATTAGGA
GTGTTTTAAAGAATGCCATAGATGTTGATTCTTAACTGCTACAGATAACCTGTAATTGAGCAGAT
TTAAAATTCAGGCATACTTTTCCATTTATCCAAGTGCTTTCATTTTTCCAGATGGCTTCAGAAGT
AGGCTCGTGGGCAGGGCGCAGACCTGATCTTTATAGGGTTGACATAGAAAGCAGTAGTTGTGGGT
GAAAGGGCAGGTTGTCTTCAAACTCTGTGAGGTAGAATCCTTTGTCTATACCTCCATGAACATTG
ACTCGTGTGTTCAGAGCCTTTGGCCTCTCTGTGGAGTCTGGCTCTCTGGCTCCTGTGCATTCTTT
GAATAGTCACTCGTAAAAACTGTCAGTGCTTGAAACTGTTTCCTTTACTCATGTTGAAGGGACTT
TGTTGGCTTTTAGAGTGTTGGTCATGACTCCAAGAGCAGAGCAGGGAAGAGCCCAAGCATAGACT
TGGTGCCGTGGTGATGGCTGCAGTCCAGTTTTGTGATGCTGCTTTTACGTGTCCCTCGATAACAG
TCAGCTAGACACACTCAGGAGGACTACTGAGGCTCTGCGACCTTCAGGAGCTGAGCCTGCCTCTC
TCCTTTAGATGACAGACCTTCATCTGGGAACGTGCTGAGCCAGCACCCTCAGATGATTTCCCTCC
AAACTGCTGACTAGGTCATCCTCTGTCTGGTAGAGACATTCACATCTTTGCTTTTATTCTATGCT
CTCTGTACTTTTGACCAAAAATTGACCAAAGTAAGAAAATGCAAGTTCTAAAAATAGACTAAGGA
TGCCTTTGCAGAACACCAAAGCATCCCAAGGAACTGGTAGGGAAGTGGCGCCTGTCTCCTGGAGT
GGAAGAGGCCTGCTCCCTGGCTCTGGGTCTGCTGGGGGCACAGTAAATCAGTCTTGGCACCCACA
TCCAGGGCAGAGAGGTCTGTGGTTCTCAGCATCAGAAGGCAGCGCAGCCCCTCTCCTCTTCAGGC
TACAGGGTTGTCACCTGCTGAGTCCTCAGGTTGTTTGGCCTCTCTGGTCCATCTTGGGCATTAGG
TTCTCCAGCAGAGCTCTGGCCAGCTGCCTCTTCTTTAACTGGGAACACAGGCTCTCACAAGATCA
GAACCCCCACTCACCCCCAAGATCTTATCTAGCAAGCCTGTAGTATTCAGTTTCTGTTGTAGGAA
GAGAGCGAGGCATCCCTGAATTCCACGCATCTGCTGGAAACGAGCCGTGTCAGATCGCACATCCC
TGCGCCCCCATGCCCCTCTGAGTCACACAGGACAGAGGAGGCAGAGCTTCTGCCCACTGTTATCT
TCACTTTCTTTGTCCAGTCTTTTGTTTTTAATAAGCAGTGACCCTCCCTACTCTTCTTTTTAATG
ATTTTTGTAGTTGATTTGTCTGAACTGTGGCTACTGTGCATTCCTTGAATAATCACTTGTAAAAA
TTGTCAGTGCTTGAAGCTGTTTCCTTTACTCACATTGAAGGGACTTCGTTGGTTTTTTGGAGTCT
TGGTTGTGACTCCAAGAGCAGAGTGAGGAAGACCCCCAAGCATAGACTCGGGTACTGTGATGATG
GCTGCAGTCCAGTTTTATGATTCTGCTTTTATGTGTCCCTTGATAACAGTGACTTAACAATATAC
ATTCCTCATAAATAAAAAAAAAACAAGAATCTGAATTCTTAGAAA
NP_001102.3
SEQ ID NO: 8
MNPRQGYSLSGYYTHPFQGYEHRQLRYQQPGPGSSPSSFLLKQIEFLKGQLPEAPVIGKQTPSLP
PSLPGLRPRFPVLLASSTRGRQVDIRGVPRGVHLRSQGLQRGFQHPSPRGRSLPQRGVDCLSSHF
QELSIYQDQEQRILKFLEELGEGKATTAHDLSGKLGTPKKEINRVLYSLAKKGKLQKEAGTPPLW
KIAVSTQAWNQHSGVVRPDGHSQGAPNSDPSLEPEDRNSTSVSEDLLEPFIAVSAQAWNQHSGVV
RPDSHSQGSPNSDPGLEPEDSNSTSALEDPLEFLDMAEIKEKICDYLFNVSDSSALNLAKNIGLT
KARDINAVLIDMERQGDVYRQGTTPPIWHLTDKKRERMQIKRNTNSVPETAPAAIPETKRNAEFL
TCNIPTSNASNNMVTTEKVENGQEPVIKLENRQEARPEPARLKPPVHYNGPSKAGYVDFENGQWA
TDDIPDDLNSIRAAPGEFRAIMEMPSFYSHGLPRCSPYKKLTECQLKNPISGLLEYAQFASQTCE
FNMIEQSGPPHEPRFKFQVVINGREFPPAEAGSKKVAKQDAAMKAMTILLEEAKAKDSGKSEESS
HYSTEKESEKTAESQTPTPSATSFFSGKSPVTTLLECMHKLGNSCEFRLLSKEGPAHEPKFQYCV
AVGAQTFPSVSAPSKKVAKQMAAEEAMKALHGEATNSMASDNQPEGMISESLDNLESMMPNKVRK
IGELVRYLNTNPVGGLLEYARSHGFAAEFKLVDQSGPPHEPKFVYQAKVGGRWFPAVCAHSKKQG
KQEAADAALRVLIGENEKAERMGFTEVTPVTGASLRRTMLLLSRSPEAQPKTLPLTGSTFHDQIA
MLSHRCENTLTNSFQPSLLGRKILAAIIMKKDSEDMGVVVSLGTGNRCVKGDSLSLKGETVNDCH
AEIISRRGFIRFLYSELMKYNSQTAKDSIFEPAKGGEKLQIKKTVSFHLYISTAPCGDGALFDKS
CSDRAMESTESRHYPVFENPKQGKLRTKVENGEGTIPVESSDIVPTWDGIRLGERLRTMSCSDKI
LRWNVLGLQGALLTHFLQPIYLKSVTLGYLFSQGHLTRAICCRVTRDGSAFEDGLRHPFIVNHPK
VGRVSIYDSKRQSGKTKETSVNWCLADGYDLEILDGTRGTVDGPRNELSRVSKKNIFLLFKKLCS
FRYRRDLLRLSYGEAKKAARDYETAKNYFKKGLKDMGYGNWISKPQEEKNFYLCPV
NM_015840.4
SEQ ID NO: 9
GAACCGGAGCCATCTTGGGCCCGGCGCGCAGACCCGCGGAGTTTCCCGTGCCGACGCCCCGGGGC
CACTTCCAGTGCGGAGTAGCGGAGGCGTGGGGGCCTCGAGGGGCTGGCGCGGCCCAGCGGTCGGG
CCAGGGTCGTGCCGCCGGCGGGTCGGGCCGGGCAATGCCTCGCGGGCGCAATGAATCCGCGGCAG
GGGTATTCCCTCAGCGGATACTACACCCATCCATTTCAAGGCTATGAGCACAGACAGCTCAGGTA
CCAGCAGCCTGGGCCAGGATCTTCCCCCAGTAGTTTCCTGCTTAAGCAAATAGAATTTCTCAAGG
GGCAGCTCCCAGAAGCACCGGTGATTGGAAAGCAGACACCGTCACTGCCACCTTCCCTCCCAGGA
CTCCGGCCAAGGTTTCCAGTACTACTTGCCTCCAGTACCAGAGGCAGGCAAGTGGACATCAGGGG
TGTCCCCAGGGGCGTGCATCTCAGAAGTCAGGGGCTCCAGAGAGGGTTCCAGCATCCTTCACCAC
GTGGCAGGAGTCTGCCACAGAGAGGTGTTGATTGCCTTTCCTCACATTTCCAGGAACTGAGTATC
TACCAAGATCAGGAACAAAGGATCTTAAAGTTCCTGGAAGAGCTTGGGGAAGGGAAGGCCACCAC
AGCACATGATCTGTCTGGGAAACTTGGGACTCCGAAGAAAGAAATCAATCGAGTTTTATACTCCC
TGGCAAAGAAGGGCAAGCTACAGAAAGAGGCAGGAACACCCCCTTTGTGGAAAATCGCGGTCTCC
ACTCAGGCTTGGAACCAGCACAGCGGAGTGGTAAGACCAGACGGTCATAGCCAAGGAGCCCCAAA
CTCAGACCCGAGTTTGGAACCGGAAGACAGAAACTCCACATCTGTCTCAGAAGATCTTCTTGAGC
CTTTTATTGCAGTCTCAGCTCAGGCTTGGAACCAGCACAGCGGAGTGGTAAGACCAGACAGTCAT
AGCCAAGGATCCCCAAACTCAGACCCAGGTTTGGAACCTGAAGACAGCAACTCCACATCTGCCTT
GGAAGATCCTCTTGAGTTTTTAGACATGGCCGAGATCAAGGAGAAAATCTGCGACTATCTCTTCA
ATGTGTCTGACTCCTCTGCCCTGAATTTGGCTAAAAATATTGGCCTTACCAAGGCCCGAGATATA
AATGCTGTGCTAATTGACATGGAAAGGCAGGGGGATGTCTATAGACAAGGGACAACCCCTCCCAT
ATGGCATTTGACAGACAAGAAGCGAGAGAGGATGCAAATCAAGAGAAATACGAACAGTGTTCCTG
AAACCGCTCCAGCTGCAATCCCTGAGACCAAAAGAAACGCAGAGTTCCTCACCTGTAATATACCC
ACATCAAATGCCTCAAATAACATGGTAACCACAGAAAAAGTGGAGAATGGGCAGGAACCTGTCAT
AAAGTTAGAAAACAGGCAAGAGGCCAGACCAGAACCAGCAAGACTGAAACCACCTGTTCATTACA
ATGGCCCCTCAAAAGCAGGGTATGTTGACTTTGAAAATGGCCAGTGGGCCACAGATGACATCCCA
GATGACTTGAATAGTATCCGCGCAGCACCAGGTGAGTTTCGAGCCATCATGGAGATGCCCTCCTT
CTACAGTCATGGCTTGCCACGGTGTTCACCCTACAAGAAACTGACAGAGTGCCAGCTGAAGAACC
CCATCAGCGGGCTGTTAGAATATGCCCAGTTCGCTAGTCAAACCTGTGAGTTCAACATGATAGAG
CAGAGTGGACCACCCCATGAACCTCGATTTAAATTCCAGGTTGTCATCAATGGCCGAGAGTTTCC
CCCAGCTGAAGCTGGAAGCAAGAAAGTGGCCAAGCAGGATGCAGCTATGAAAGCCATGACAATTC
TGCTAGAGGAAGCCAAAGCCAAGGACAGTGGAAAATCAGAAGAATCATCCCACTATTCCACAGAG
AAAGAATCAGAGAAGACTGCAGAGTCCCAGACCCCCACCCCTTCAGCCACATCCTTCTTTTCTGG
GAAGAGCCCCGTCACCACACTGCTTGAGTGTATGCACAAATTGGGGAACTCCTGCGAATTCCGTC
TCCTGTCCAAAGAAGGCCCTGCCCATGAACCCAAGTTCCAATACTGTGTTGCAGTGGGAGCCCAA
ACTTTCCCCAGTGTGAGTGCTCCCAGCAAGAAAGTGGCAAAGCAGATGGCCGCAGAGGAAGCCAT
GAAGGCCCTGCATGGGGAGGCGACCAACTCCATGGCTTCTGATAACCAGCCTGAAGGTATGATCT
CAGAGTCACTTGATAACTTGGAATCCATGATGCCCAACAAGGTCAGGAAGATTGGCGAGCTCGTG
AGATACCTGAACACCAACCCTGTGGGTGGCCTTTTGGAGTACGCCCGCTCCCATGGCTTTGCTGC
TGAATTCAAGTTGGTCGACCAGTCCGGACCTCCTCACGAGCCCAAGTTCGTTTACCAAGCAAAAG
TTGGGGGTCGCTGGTTCCCAGCCGTCTGCGCACACAGCAAGAAGCAAGGCAAGCAGGAAGCAGCA
GATGCGGCTCTCCGTGTCTTGATTGGGGAGAACGAGAAGGCAGAACGCATGGGTTTCACAGAGCT
CCCTCTCACTGGCAGCACCTTCCATGACCAGATAGCCATGCTGAGCCACCGGTGCTTCAACACTC
TGACTAACAGCTTCCAGCCCTCCTTGCTCGGCCGCAAGATTCTGGCCGCCATCATTATGAAAAAA
GACTCTGAGGACATGGGTGTCGTCGTCAGCTTGGGAACAGGGAATCGCTGTGTGAAAGGAGATTC
TCTCAGCCTAAAAGGAGAAACTGTCAATGACTGCCATGCAGAAATAATCTCCCGGAGAGGCTTCA
TCAGGTTTCTCTACAGTGAGTTAATGAAATACAACTCCCAGACTGCGAAGGATAGTATATTTGAA
CCTGCTAAGGGAGGAGAAAAGCTCCAAATAAAAAAGACTGTGTCATTCCATCTGTATATCAGCAC
TGCTCCGTGTGGAGATGGCGCCCTCTTTGACAAGTCCTGCAGCGACCGTGCTATGGAAAGCACAG
AATCCCGCCACTACCCTGTCTTCGAGAATCCCAAACAAGGAAAGCTCCGCACCAAGGTGGAGAAC
GGAGAAGGCACAATCCCTGTGGAATCCAGTGACATTGTGCCTACGTGGGATGGCATTCGGCTCGG
GGAGAGACTCCGTACCATGTCCTGTAGTGACAAAATCCTACGCTGGAACGTGCTGGGCCTGCAAG
GGGCACTGTTGACCCACTTCCTGCAGCCCATTTATCTCAAATCTGTCACATTGGGTTACCTTTTC
AGCCAAGGGCATCTGACCCGTGCTATTTGCTGTCGTGTGACAAGAGATGGGAGTGCATTTGAGGA
TGGACTACGACATCCCTTTATTGTCAACCACCCCAAGGTTGGCAGAGTCAGCATATATGATTCCA
AAAGGCAATCCGGGAAGACTAAGGAGACAAGCGTCAACTGGTGTCTGGCTGATGGCTATGACCTG
GAGATCCTGGACGGTACCAGAGGCACTGTGGATGGGCCACGGAATGAATTGTCCCGGGTCTCCAA
AAAGAACATTTTTCTTCTATTTAAGAAGCTCTGCTCCTTCCGTTACCGCAGGGATCTACTGAGAC
TCTCCTATGGTGAGGCCAAGAAAGCTGCCCGTGACTACGAGACGGCCAAGAACTACTTCAAAAAA
GGCCTGAAGGATATGGGCTATGGGAACTGGATTAGCAAACCCCAGGAGGAAAAGAACTTTTATCT
CTGCCCAGTATAGTATGCTCCAGTGACAGATGGATTAGGGTGTGTCATACTAGGGTGTGAGAGAG
GTAGGTCGTAGCATTCCTCATCACATGGTCAGGGGATTTTTTTTTCTCCTTTTTTTTTCTTTTTA
AGCCATAATTGGTGATACTGAAAACTTTGGGTTCCCATTTATCCTGCTTTCTTTGGGATTGCTAG
GCAAGGTCTGGCCAGGCCCCCCTTTTTTCCCCCAAGTGAAGAGGCAGAAACCTAAGAAGTTATCT
TTTCTTTCTACCCAAAGCATACATAGTCACTGAGCACCTGCGGTCCATTTCCTCTTAAAAGTTTT
GTTTTGATTTGTTTCCATTTCCTTTCCCTTTGTGTTTGCTACACTGACCTCTTGCGGTCTTGATT
AGGTTTCAGTCAACTCTGGATCATGTCAGGGACTGATAATTTCATTTGTGGATTACGCAGACCCC
TCTACTTCCCCTCTTTCCCTTCTGAGATTCTTTCCTTGTGATCTGAATGTCTCCTTTTCCCCCTC
AGAGGGCAAAGAGGTGAACATAAAGGATTTGGTGAAACATTTGTAAGGGTAGGAGTTGAAAACTG
CAGTTCCCAGTGCCACGGAAGTGTGATTGGAGCCTGCAGATAATGCCCAGCCATCCTCCCATCCT
GCACTTTAGCCAGCTGCAGGGCGGGCAAGGCAAGGAAAGCTGCTTCCCTGGAAGTGTATCACTTT
CTCCGGCAGCTGGGAAGTCTAGAACCAGCCAGACTGGGTTAAGGGAGCTGCTCAAGCAATAGCAG
AGGTTTCACCCGGCAGGATGACACAGACCACTTCCCAGGGAGCACGGGCATGCCTTGGAATATTG
CCAAGCTTCCAGCTGCCTCTTCTCCTAAAGCATTCCTAGGAATATTTTCCCCGCCAATGCTGGGC
GTACACCCTAGCCAACGGGACAAATCCTAGAGGGTATAAAATCATCTCTGCTCAGATAATCATGA
CTTAGCAAGAATAAGGGCAAAAAATCCTGTTGGCTTAACGTCACTGTTCCACCCGGTGTAATATC
TCTCATGACAGTGACACCAAGGGAAGTTGACTAAGTCACATGTAAATTAGGAGTGTTTTAAAGAA
TGCCATAGATGTTGATTCTTAACTGCTACAGATAACCTGTAATTGAGCAGATTTAAAATTCAGGC
ATACTTTTCCATTTATCCAAGTGCTTTCATTTTTCCAGATGGCTTCAGAAGTAGGCTCGTGGGCA
GGGCGCAGACCTGATCTTTATAGGGTTGACATAGAAAGCAGTAGTTGTGGGTGAAAGGGCAGGTT
GTCTTCAAACTCTGTGAGGTAGAATCCTTTGTCTATACCTCCATGAACATTGACTCGTGTGTTCA
GAGCCTTTGGCCTCTCTGTGGAGTCTGGCTCTCTGGCTCCTGTGCATTCTTTGAATAGTCACTCG
TAAAAACTGTCAGTGCTTGAAACTGTTTCCTTTACTCATGTTGAAGGGACTTTGTTGGCTTTTAG
AGTGTTGGTCATGACTCCAAGAGCAGAGCAGGGAAGAGCCCAAGCATAGACTTGGTGCCGTGGTG
ATGGCTGCAGTCCAGTTTTGTGATGCTGCTTTTACGTGTCCCTCGATAACAGTCAGCTAGACACA
CTCAGGAGGACTACTGAGGCTCTGCGACCTTCAGGAGCTGAGCCTGCCTCTCTCCTTTAGATGAC
AGACCTTCATCTGGGAACGTGCTGAGCCAGCACCCTCAGATGATTTCCCTCCAAACTGCTGACTA
GGTCATCCTCTGTCTGGTAGAGACATTCACATCTTTGCTTTTATTCTATGCTCTCTGTACTTTTG
ACCAAAAATTGACCAAAGTAAGAAAATGCAAGTTCTAAAAATAGACTAAGGATGCCTTTGCAGAA
CACCAAAGCATCCCAAGGAACTGGTAGGGAAGTGGCGCCTGTCTCCTGGAGTGGAAGAGGCCTGC
TCCCTGGCTCTGGGTCTGCTGGGGGCACAGTAAATCAGTCTTGGCACCCACATCCAGGGCAGAGA
GGTCTGTGGTTCTCAGCATCAGAAGGCAGCGCAGCCCCTCTCCTCTTCAGGCTACAGGGTTGTCA
CCTGCTGAGTCCTCAGGTTGTTTGGCCTCTCTGGTCCATCTTGGGCATTAGGTTCTCCAGCAGAG
CTCTGGCCAGCTGCCTCTTCTTTAACTGGGAACACAGGCTCTCACAAGATCAGAACCCCCACTCA
CCCCCAAGATCTTATCTAGCAAGCCTGTAGTATTCAGTTTCTGTTGTAGGAAGAGAGCGAGGCAT
CCCTGAATTCCACGCATCTGCTGGAAACGAGCCGTGTCAGATCGCACATCCCTGCGCCCCCATGC
CCCTCTGAGTCACACAGGACAGAGGAGGCAGAGCTTCTGCCCACTGTTATCTTCACTTTCTTTGT
CCAGTCTTTTGTTTTTAATAAGCAGTGACCCTCCCTACTCTTCTTTTTAATGATTTTTGTAGTTG
ATTTGTCTGAACTGTGGCTACTGTGCATTCCTTGAATAATCACTTGTAAAAATTGTCAGTGCTTG
AAGCTGTTTCCTTTACTCACATTGAAGGGACTTCGTTGGTTTTTTGGAGTCTTGGTTGTGACTCC
AAGAGCAGAGTGAGGAAGACCCCCAAGCATAGACTCGGGTACTGTGATGATGGCTGCAGTCCAGT
TTTATGATTCTGCTTTTATGTGTCCCTTGATAACAGTGACTTAACAATATACATTCCTCATAAAT
AAAAAAAAAACAAGAATCTGAATTCTTAGAAA
NP_056655.3
SEQ ID NO: 10
MNPRQGYSLSGYYTHPFQGYEHRQLRYQQPGPGSSPSSFLLKQIEFLKGQLPEAPVIGKQTPSLP
PSLPGLRPRFPVLLASSTRGRQVDIRGVPRGVHLRSQGLQRGFQHPSPRGRSLPQRGVDCLSSHF
QELSIYQDQEQRILKFLEELGEGKATTAHDLSGKLGTPKKEINRVLYSLAKKGKLQKEAGTPPLW
KIAVSTQAWNQHSGVVRPDGHSQGAPNSDPSLEPEDRNSTSVSEDLLEPFIAVSAQAWNQHSGVV
RPDSHSQGSPNSDPGLEPEDSNSTSALEDPLEFLDMAEIKEKICDYLFNVSDSSALNLAKNIGLT
KARDINAVLIDMERQGDVYRQGTTPPIWHLTDKKRERMQIKRNTNSVPETAPAAIPETKRNAEFL
TCNIPTSNASNNMVTTEKVENGQEPVIKLENRQEARPEPARLKPPVHYNGPSKAGYVDFENGQWA
TDDIPDDLNSIRAAPGEFRAIMEMPSFYSHGLPRCSPYKKLTECQLKNPISGLLEYAQFASQTCE
FNMIEQSGPPHEPRFKFQVVINGREFPPAEAGSKKVAKQDAAMKAMTILLEEAKAKDSGKSEESS
HYSTEKESEKTAESQTPTPSATSFFSGKSPVTTLLECMHKLGNSCEFRLLSKEGPAHEPKFQYCV
AVGAQTFPSVSAPSKKVAKQMAAEEAMKALHGEATNSMASDNQPEGMISESLDNLESMMPNKVRK
IGELVRYLNTNPVGGLLEYARSHGFAAEFKLVDQSGPPHEPKFVYQAKVGGRWFPAVCAHSKKQG
KQEAADAALRVLIGENEKAERMGFTELPLTGSTFHDQIAMLSHRCENTLTNSFQPSLLGRKILAA
IIMKKDSEDMGVVVSLGTGNRCVKGDSLSLKGETVNDCHAEIISRRGFIRFLYSELMKYNSQTAK
DSIFEPAKGGEKLQIKKTVSFHLYISTAPCGDGALFDKSCSDRAMESTESRHYPVFENPKQGKLR
TKVENGEGTIPVESSDIVPTWDGIRLGERLRTMSCSDKILRWNVLGLQGALLTHFLQPIYLKSVT
LGYLFSQGHLTRAICCRVTRDGSAFEDGLRHPFIVNHPKVGRVSIYDSKRQSGKTKETSVNWCLA
DGYDLEILDGTRGTVDGPRNELSRVSKKNIFLLFKKLCSFRYRRDLLRLSYGEAKKAARDYETAK
NYFKKGLKDMGYGNWISKPQEEKNFYLCPV
NM_015841.4
SEQ ID NO: 11
GAACCGGAGCCATCTTGGGCCCGGCGCGCAGACCCGCGGAGTTTCCCGTGCCGACGCCCCGGGGC
CACTTCCAGTGCGGAGTAGCGGAGGCGTGGGGGCCTCGAGGGGCTGGCGCGGCCCAGCGGTCGGG
CCAGGGTCGTGCCGCCGGCGGGTCGGGCCGGGCAATGCCTCGCGGGCGCAATGAATCCGCGGCAG
GGGTATTCCCTCAGCGGATACTACACCCATCCATTTCAAGGCTATGAGCACAGACAGCTCAGGTA
CCAGCAGCCTGGGCCAGGATCTTCCCCCAGTAGTTTCCTGCTTAAGCAAATAGAATTTCTCAAGG
GGCAGCTCCCAGAAGCACCGGTGATTGGAAAGCAGACACCGTCACTGCCACCTTCCCTCCCAGGA
CTCCGGCCAAGGTTTCCAGTACTACTTGCCTCCAGTACCAGAGGCAGGCAAGTGGACATCAGGGG
TGTCCCCAGGGGCGTGCATCTCAGAAGTCAGGGGCTCCAGAGAGGGTTCCAGCATCCTTCACCAC
GTGGCAGGAGTCTGCCACAGAGAGGTGTTGATTGCCTTTCCTCACATTTCCAGGAACTGAGTATC
TACCAAGATCAGGAACAAAGGATCTTAAAGTTCCTGGAAGAGCTTGGGGAAGGGAAGGCCACCAC
AGCACATGATCTGTCTGGGAAACTTGGGACTCCGAAGAAAGAAATCAATCGAGTTTTATACTCCC
TGGCAAAGAAGGGCAAGCTACAGAAAGAGGCAGGAACACCCCCTTTGTGGAAAATCGCGGTCTCC
ACTCAGGCTTGGAACCAGCACAGCGGAGTGGTAAGACCAGACGGTCATAGCCAAGGAGCCCCAAA
CTCAGACCCGAGTTTGGAACCGGAAGACAGAAACTCCACATCTGTCTCAGAAGATCTTCTTGAGC
CTTTTATTGCAGTCTCAGCTCAGGCTTGGAACCAGCACAGCGGAGTGGTAAGACCAGACAGTCAT
AGCCAAGGATCCCCAAACTCAGACCCAGGTTTGGAACCTGAAGACAGCAACTCCACATCTGCCTT
GGAAGATCCTCTTGAGTTTTTAGACATGGCCGAGATCAAGGAGAAAATCTGCGACTATCTCTTCA
ATGTGTCTGACTCCTCTGCCCTGAATTTGGCTAAAAATATTGGCCTTACCAAGGCCCGAGATATA
AATGCTGTGCTAATTGACATGGAAAGGCAGGGGGATGTCTATAGACAAGGGACAACCCCTCCCAT
ATGGCATTTGACAGACAAGAAGCGAGAGAGGATGCAAATCAAGAGAAATACGAACAGTGTTCCTG
AAACCGCTCCAGCTGCAATCCCTGAGACCAAAAGAAACGCAGAGTTCCTCACCTGTAATATACCC
ACATCAAATGCCTCAAATAACATGGTAACCACAGAAAAAGTGGAGAATGGGCAGGAACCTGTCAT
AAAGTTAGAAAACAGGCAAGAGGCCAGACCAGAACCAGCAAGACTGAAACCACCTGTTCATTACA
ATGGCCCCTCAAAAGCAGGGTATGTTGACTTTGAAAATGGCCAGTGGGCCACAGATGACATCCCA
GATGACTTGAATAGTATCCGCGCAGCACCAGGTGAGTTTCGAGCCATCATGGAGATGCCCTCCTT
CTACAGTCATGGCTTGCCACGGTGTTCACCCTACAAGAAACTGACAGAGTGCCAGCTGAAGAACC
CCATCAGCGGGCTGTTAGAATATGCCCAGTTCGCTAGTCAAACCTGTGAGTTCAACATGATAGAG
CAGAGTGGACCACCCCATGAACCTCGATTTAAATTCCAGGTTGTCATCAATGGCCGAGAGTTTCC
CCCAGCTGAAGCTGGAAGCAAGAAAGTGGCCAAGCAGGATGCAGCTATGAAAGCCATGACAATTC
TGCTAGAGGAAGCCAAAGCCAAGGACAGTGGAAAATCAGAAGAATCATCCCACTATTCCACAGAG
AAAGAATCAGAGAAGACTGCAGAGTCCCAGACCCCCACCCCTTCAGCCACATCCTTCTTTTCTGG
GAAGAGCCCCGTCACCACACTGCTTGAGTGTATGCACAAATTGGGGAACTCCTGCGAATTCCGTC
TCCTGTCCAAAGAAGGCCCTGCCCATGAACCCAAGTTCCAATACTGTGTTGCAGTGGGAGCCCAA
ACTTTCCCCAGTGTGAGTGCTCCCAGCAAGAAAGTGGCAAAGCAGATGGCCGCAGAGGAAGCCAT
GAAGGCCCTGCATGGGGAGGCGACCAACTCCATGGCTTCTGATAACCAGGTCAGGAAGATTGGCG
AGCTCGTGAGATACCTGAACACCAACCCTGTGGGTGGCCTTTTGGAGTACGCCCGCTCCCATGGC
TTTGCTGCTGAATTCAAGTTGGTCGACCAGTCCGGACCTCCTCACGAGCCCAAGTTCGTTTACCA
AGCAAAAGTTGGGGGTCGCTGGTTCCCAGCCGTCTGCGCACACAGCAAGAAGCAAGGCAAGCAGG
AAGCAGCAGATGCGGCTCTCCGTGTCTTGATTGGGGAGAACGAGAAGGCAGAACGCATGGGTTTC
ACAGAGCTCCCTCTCACTGGCAGCACCTTCCATGACCAGATAGCCATGCTGAGCCACCGGTGCTT
CAACACTCTGACTAACAGCTTCCAGCCCTCCTTGCTCGGCCGCAAGATTCTGGCCGCCATCATTA
TGAAAAAAGACTCTGAGGACATGGGTGTCGTCGTCAGCTTGGGAACAGGGAATCGCTGTGTGAAA
GGAGATTCTCTCAGCCTAAAAGGAGAAACTGTCAATGACTGCCATGCAGAAATAATCTCCCGGAG
AGGCTTCATCAGGTTTCTCTACAGTGAGTTAATGAAATACAACTCCCAGACTGCGAAGGATAGTA
TATTTGAACCTGCTAAGGGAGGAGAAAAGCTCCAAATAAAAAAGACTGTGTCATTCCATCTGTAT
ATCAGCACTGCTCCGTGTGGAGATGGCGCCCTCTTTGACAAGTCCTGCAGCGACCGTGCTATGGA
AAGCACAGAATCCCGCCACTACCCTGTCTTCGAGAATCCCAAACAAGGAAAGCTCCGCACCAAGG
TGGAGAACGGAGAAGGCACAATCCCTGTGGAATCCAGTGACATTGTGCCTACGTGGGATGGCATT
CGGCTCGGGGAGAGACTCCGTACCATGTCCTGTAGTGACAAAATCCTACGCTGGAACGTGCTGGG
CCTGCAAGGGGCACTGTTGACCCACTTCCTGCAGCCCATTTATCTCAAATCTGTCACATTGGGTT
ACCTTTTCAGCCAAGGGCATCTGACCCGTGCTATTTGCTGTCGTGTGACAAGAGATGGGAGTGCA
TTTGAGGATGGACTACGACATCCCTTTATTGTCAACCACCCCAAGGTTGGCAGAGTCAGCATATA
TGATTCCAAAAGGCAATCCGGGAAGACTAAGGAGACAAGCGTCAACTGGTGTCTGGCTGATGGCT
ATGACCTGGAGATCCTGGACGGTACCAGAGGCACTGTGGATGGGCCACGGAATGAATTGTCCCGG
GTCTCCAAAAAGAACATTTTTCTTCTATTTAAGAAGCTCTGCTCCTTCCGTTACCGCAGGGATCT
ACTGAGACTCTCCTATGGTGAGGCCAAGAAAGCTGCCCGTGACTACGAGACGGCCAAGAACTACT
TCAAAAAAGGCCTGAAGGATATGGGCTATGGGAACTGGATTAGCAAACCCCAGGAGGAAAAGAAC
TTTTATCTCTGCCCAGTATAGTATGCTCCAGTGACAGATGGATTAGGGTGTGTCATACTAGGGTG
TGAGAGAGGTAGGTCGTAGCATTCCTCATCACATGGTCAGGGGATTTTTTTTTCTCCTTTTTTTT
TCTTTTTAAGCCATAATTGGTGATACTGAAAACTTTGGGTTCCCATTTATCCTGCTTTCTTTGGG
ATTGCTAGGCAAGGTCTGGCCAGGCCCCCCTTTTTTCCCCCAAGTGAAGAGGCAGAAACCTAAGA
AGTTATCTTTTCTTTCTACCCAAAGCATACATAGTCACTGAGCACCTGCGGTCCATTTCCTCTTA
AAAGTTTTGTTTTGATTTGTTTCCATTTCCTTTCCCTTTGTGTTTGCTACACTGACCTCTTGCGG
TCTTGATTAGGTTTCAGTCAACTCTGGATCATGTCAGGGACTGATAATTTCATTTGTGGATTACG
CAGACCCCTCTACTTCCCCTCTTTCCCTTCTGAGATTCTTTCCTTGTGATCTGAATGTCTCCTTT
TCCCCCTCAGAGGGCAAAGAGGTGAACATAAAGGATTTGGTGAAACATTTGTAAGGGTAGGAGTT
GAAAACTGCAGTTCCCAGTGCCACGGAAGTGTGATTGGAGCCTGCAGATAATGCCCAGCCATCCT
CCCATCCTGCACTTTAGCCAGCTGCAGGGCGGGCAAGGCAAGGAAAGCTGCTTCCCTGGAAGTGT
ATCACTTTCTCCGGCAGCTGGGAAGTCTAGAACCAGCCAGACTGGGTTAAGGGAGCTGCTCAAGC
AATAGCAGAGGTTTCACCCGGCAGGATGACACAGACCACTTCCCAGGGAGCACGGGCATGCCTTG
GAATATTGCCAAGCTTCCAGCTGCCTCTTCTCCTAAAGCATTCCTAGGAATATTTTCCCCGCCAA
TGCTGGGCGTACACCCTAGCCAACGGGACAAATCCTAGAGGGTATAAAATCATCTCTGCTCAGAT
AATCATGACTTAGCAAGAATAAGGGCAAAAAATCCTGTTGGCTTAACGTCACTGTTCCACCCGGT
GTAATATCTCTCATGACAGTGACACCAAGGGAAGTTGACTAAGTCACATGTAAATTAGGAGTGTT
TTAAAGAATGCCATAGATGTTGATTCTTAACTGCTACAGATAACCTGTAATTGAGCAGATTTAAA
ATTCAGGCATACTTTTCCATTTATCCAAGTGCTTTCATTTTTCCAGATGGCTTCAGAAGTAGGCT
CGTGGGCAGGGCGCAGACCTGATCTTTATAGGGTTGACATAGAAAGCAGTAGTTGTGGGTGAAAG
GGCAGGTTGTCTTCAAACTCTGTGAGGTAGAATCCTTTGTCTATACCTCCATGAACATTGACTCG
TGTGTTCAGAGCCTTTGGCCTCTCTGTGGAGTCTGGCTCTCTGGCTCCTGTGCATTCTTTGAATA
GTCACTCGTAAAAACTGTCAGTGCTTGAAACTGTTTCCTTTACTCATGTTGAAGGGACTTTGTTG
GCTTTTAGAGTGTTGGTCATGACTCCAAGAGCAGAGCAGGGAAGAGCCCAAGCATAGACTTGGTG
CCGTGGTGATGGCTGCAGTCCAGTTTTGTGATGCTGCTTTTACGTGTCCCTCGATAACAGTCAGC
TAGACACACTCAGGAGGACTACTGAGGCTCTGCGACCTTCAGGAGCTGAGCCTGCCTCTCTCCTT
TAGATGACAGACCTTCATCTGGGAACGTGCTGAGCCAGCACCCTCAGATGATTTCCCTCCAAACT
GCTGACTAGGTCATCCTCTGTCTGGTAGAGACATTCACATCTTTGCTTTTATTCTATGCTCTCTG
TACTTTTGACCAAAAATTGACCAAAGTAAGAAAATGCAAGTTCTAAAAATAGACTAAGGATGCCT
TTGCAGAACACCAAAGCATCCCAAGGAACTGGTAGGGAAGTGGCGCCTGTCTCCTGGAGTGGAAG
AGGCCTGCTCCCTGGCTCTGGGTCTGCTGGGGGCACAGTAAATCAGTCTTGGCACCCACATCCAG
GGCAGAGAGGTCTGTGGTTCTCAGCATCAGAAGGCAGCGCAGCCCCTCTCCTCTTCAGGCTACAG
GGTTGTCACCTGCTGAGTCCTCAGGTTGTTTGGCCTCTCTGGTCCATCTTGGGCATTAGGTTCTC
CAGCAGAGCTCTGGCCAGCTGCCTCTTCTTTAACTGGGAACACAGGCTCTCACAAGATCAGAACC
CCCACTCACCCCCAAGATCTTATCTAGCAAGCCTGTAGTATTCAGTTTCTGTTGTAGGAAGAGAG
CGAGGCATCCCTGAATTCCACGCATCTGCTGGAAACGAGCCGTGTCAGATCGCACATCCCTGCGC
CCCCATGCCCCTCTGAGTCACACAGGACAGAGGAGGCAGAGCTTCTGCCCACTGTTATCTTCACT
TTCTTTGTCCAGTCTTTTGTTTTTAATAAGCAGTGACCCTCCCTACTCTTCTTTTTAATGATTTT
TGTAGTTGATTTGTCTGAACTGTGGCTACTGTGCATTCCTTGAATAATCACTTGTAAAAATTGTC
AGTGCTTGAAGCTGTTTCCTTTACTCACATTGAAGGGACTTCGTTGGTTTTTTGGAGTCTTGGTT
GTGACTCCAAGAGCAGAGTGAGGAAGACCCCCAAGCATAGACTCGGGTACTGTGATGATGGCTGC
AGTCCAGTTTTATGATTCTGCTTTTATGTGTCCCTTGATAACAGTGACTTAACAATATACATTCC
TCATAAATAAAAAAAAAACAAGAATCTGAATTCTTAGAAA
NP_056656.3
SEQ ID NO: 12
MNPRQGYSLSGYYTHPFQGYEHRQLRYQQPGPGSSPSSFLLKQIEFLKGQLPEAPVIGKQTPSLP
PSLPGLRPRFPVLLASSTRGRQVDIRGVPRGVHLRSQGLQRGFQHPSPRGRSLPQRGVDCLSSHF
QELSIYQDQEQRILKFLEELGEGKATTAHDLSGKLGTPKKEINRVLYSLAKKGKLQKEAGTPPLW
KIAVSTQAWNQHSGVVRPDGHSQGAPNSDPSLEPEDRNSTSVSEDLLEPFIAVSAQAWNQHSGVV
RPDSHSQGSPNSDPGLEPEDSNSTSALEDPLEFLDMAEIKEKICDYLFNVSDSSALNLAKNIGLT
KARDINAVLIDMERQGDVYRQGTTPPIWHLTDKKRERMQIKRNTNSVPETAPAAIPETKRNAEFL
TCNIPTSNASNNMVTTEKVENGQEPVIKLENRQEARPEPARLKPPVHYNGPSKAGYVDFENGQWA
TDDIPDDLNSIRAAPGEFRAIMEMPSFYSHGLPRCSPYKKLTECQLKNPISGLLEYAQFASQTCE
FNMIEQSGPPHEPRFKFQVVINGREFPPAEAGSKKVAKQDAAMKAMTILLEEAKAKDSGKSEESS
HYSTEKESEKTAESQTPTPSATSFFSGKSPVTTLLECMHKLGNSCEFRLLSKEGPAHEPKFQYCV
AVGAQTFPSVSAPSKKVAKQMAAEEAMKALHGEATNSMASDNQVRKIGELVRYLNTNPVGGLLEY
ARSHGFAAEFKLVDQSGPPHEPKFVYQAKVGGRWFPAVCAHSKKQGKQEAADAALRVLIGENEKA
ERMGFTELPLTGSTFHDQIAMLSHRCFNTLTNSFQPSLLGRKILAAIIMKKDSEDMGVVVSLGTG
NRCVKGDSLSLKGETVNDCHAEIISRRGFIRFLYSELMKYNSQTAKDSIFEPAKGGEKLQIKKTV
SFHLYISTAPCGDGALFDKSCSDRAMESTESRHYPVFENPKQGKLRTKVENGEGTIPVESSDIVP
TWDGIRLGERLRTMSCSDKILRWNVLGLQGALLTHFLQPIYLKSVTLGYLFSQGHLTRAICCRVT
RDGSAFEDGLRHPFIVNHPKVGRVSIYDSKRQSGKTKETSVNWCLADGYDLEILDGTRGTVDGPR
NELSRVSKKNIFLLFKKLCSFRYRRDLLRLSYGEAKKAARDYETAKNYFKKGLKDMGYGNWISKP
QEEKNFYLCPV
NM_001365045.1
SEQ ID NO: 13
TGATGTCACCAATCTGCGACCAGACCATTGATTCCCGACTGAAGGGGTATTCCCTCAGCGGATAC
TACACCCATCCATTTCAAGGCTATGAGCACAGACAGCTCAGGTACCAGCAGCCTGGGCCAGGATC
TTCCCCCAGTAGTTTCCTGCTTAAGCAAATAGAATTTCTCAAGGGGCAGCTCCCAGAAGCACCGG
TGATTGGAAAGCAGACACCGTCACTGCCACCTTCCCTCCCAGGACTCCGGCCAAGGTTTCCAGTA
CTACTTGCCTCCAGTACCAGAGGCAGGCAAGTGGACATCAGGGGTGTCCCCAGGGGCGTGCATCT
CAGAAGTCAGGGGCTCCAGAGAGGGTTCCAGCATCCTTCACCACGTGGCAGGAGTCTGCCACAGA
GAGGTGTTGATTGCCTTTCCTCACATTTCCAGGAACTGAGTATCTACCAAGATCAGGAACAAAGG
ATCTTAAAGTTCCTGGAAGAGCTTGGGGAAGGGAAGGCCACCACAGCACATGATCTGTCTGGGAA
ACTTGGGACTCCGAAGAAAGAAATCAATCGAGTTTTATACTCCCTGGCAAAGAAGGGCAAGCTAC
AGAAAGAGGCAGGAACACCCCCTTTGTGGAAAATCGCGGTCTCCACTCAGGCTTGGAACCAGCAC
AGCGGAGTGGTAAGACCAGACGGTCATAGCCAAGGAGCCCCAAACTCAGACCCGAGTTTGGAACC
GGAAGACAGAAACTCCACATCTGTCTCAGAAGATCTTCTTGAGCCTTTTATTGCAGTCTCAGCTC
AGGCTTGGAACCAGCACAGCGGAGTGGTAAGACCAGACAGTCATAGCCAAGGATCCCCAAACTCA
GACCCAGGTTTGGAACCTGAAGACAGCAACTCCACATCTGCCTTGGAAGATCCTCTTGAGTTTTT
AGACATGGCCGAGATCAAGGAGAAAATCTGCGACTATCTCTTCAATGTGTCTGACTCCTCTGCCC
TGAATTTGGCTAAAAATATTGGCCTTACCAAGGCCCGAGATATAAATGCTGTGCTAATTGACATG
GAAAGGCAGGGGGATGTCTATAGACAAGGGACAACCCCTCCCATATGGCATTTGACAGACAAGAA
GCGAGAGAGGATGCAAATCAAGAGAAATACGAACAGTGTTCCTGAAACCGCTCCAGCTGCAATCC
CTGAGACCAAAAGAAACGCAGAGTTCCTCACCTGTAATATACCCACATCAAATGCCTCAAATAAC
ATGGTAACCACAGAAAAAGTGGAGAATGGGCAGGAACCTGTCATAAAGTTAGAAAACAGGCAAGA
GGCCAGACCAGAACCAGCAAGACTGAAACCACCTGTTCATTACAATGGCCCCTCAAAAGCAGGGT
ATGTTGACTTTGAAAATGGCCAGTGGGCCACAGATGACATCCCAGATGACTTGAATAGTATCCGC
GCAGCACCAGGTGAGTTTCGAGCCATCATGGAGATGCCCTCCTTCTACAGTCATGGCTTGCCACG
GTGTTCACCCTACAAGAAACTGACAGAGTGCCAGCTGAAGAACCCCATCAGCGGGCTGTTAGAAT
ATGCCCAGTTCGCTAGTCAAACCTGTGAGTTCAACATGATAGAGCAGAGTGGACCACCCCATGAA
CCTCGATTTAAATTCCAGGTTGTCATCAATGGCCGAGAGTTTCCCCCAGCTGAAGCTGGAAGCAA
GAAAGTGGCCAAGCAGGATGCAGCTATGAAAGCCATGACAATTCTGCTAGAGGAAGCCAAAGCCA
AGGACAGTGGAAAATCAGAAGAATCATCCCACTATTCCACAGAGAAAGAATCAGAGAAGACTGCA
GAGTCCCAGACCCCCACCCCTTCAGCCACATCCTTCTTTTCTGGGAAGAGCCCCGTCACCACACT
GCTTGAGTGTATGCACAAATTGGGGAACTCCTGCGAATTCCGTCTCCTGTCCAAAGAAGGCCCTG
CCCATGAACCCAAGTTCCAATACTGTGTTGCAGTGGGAGCCCAAACTTTCCCCAGTGTGAGTGCT
CCCAGCAAGAAAGTGGCAAAGCAGATGGCCGCAGAGGAAGCCATGAAGGCCCTGCATGGGGAGGC
GACCAACTCCATGGCTTCTGATAACCAGCCTGAAGGTATGATCTCAGAGTCACTTGATAACTTGG
AATCCATGATGCCCAACAAGGTCAGGAAGATTGGCGAGCTCGTGAGATACCTGAACACCAACCCT
GTGGGTGGCCTTTTGGAGTACGCCCGCTCCCATGGCTTTGCTGCTGAATTCAAGTTGGTCGACCA
GTCCGGACCTCCTCACGAGCCCAAGTTCGTTTACCAAGCAAAAGTTGGGGGTCGCTGGTTCCCAG
CCGTCTGCGCACACAGCAAGAAGCAAGGCAAGCAGGAAGCAGCAGATGCGGCTCTCCGTGTCTTG
ATTGGGGAGAACGAGAAGGCAGAACGCATGGGTTTCACAGAGGTAACCCCAGTGACAGGGGCCAG
TCTCAGAAGAACTATGCTCCTCCTCTCAAGGTCCCCAGAAGCACAGCCAAAGACACTCCCTCTCA
CTGGCAGCACCTTCCATGACCAGATAGCCATGCTGAGCCACCGGTGCTTCAACACTCTGACTAAC
AGCTTCCAGCCCTCCTTGCTCGGCCGCAAGATTCTGGCCGCCATCATTATGAAAAAAGACTCTGA
GGACATGGGTGTCGTCGTCAGCTTGGGAACAGGGAATCGCTGTGTGAAAGGAGATTCTCTCAGCC
TAAAAGGAGAAACTGTCAATGACTGCCATGCAGAAATAATCTCCCGGAGAGGCTTCATCAGGTTT
CTCTACAGTGAGTTAATGAAATACAACTCCCAGACTGCGAAGGATAGTATATTTGAACCTGCTAA
GGGAGGAGAAAAGCTCCAAATAAAAAAGACTGTGTCATTCCATCTGTATATCAGCACTGCTCCGT
GTGGAGATGGCGCCCTCTTTGACAAGTCCTGCAGCGACCGTGCTATGGAAAGCACAGAATCCCGC
CACTACCCTGTCTTCGAGAATCCCAAACAAGGAAAGCTCCGCACCAAGGTGGAGAACGGAGAAGG
CACAATCCCTGTGGAATCCAGTGACATTGTGCCTACGTGGGATGGCATTCGGCTCGGGGAGAGAC
TCCGTACCATGTCCTGTAGTGACAAAATCCTACGCTGGAACGTGCTGGGCCTGCAAGGGGCACTG
TTGACCCACTTCCTGCAGCCCATTTATCTCAAATCTGTCACATTGGGTTACCTTTTCAGCCAAGG
GCATCTGACCCGTGCTATTTGCTGTCGTGTGACAAGAGATGGGAGTGCATTTGAGGATGGACTAC
GACATCCCTTTATTGTCAACCACCCCAAGGTTGGCAGAGTCAGCATATATGATTCCAAAAGGCAA
TCCGGGAAGACTAAGGAGACAAGCGTCAACTGGTGTCTGGCTGATGGCTATGACCTGGAGATCCT
GGACGGTACCAGAGGCACTGTGGATGGGCCACGGAATGAATTGTCCCGGGTCTCCAAAAAGAACA
TTTTTCTTCTATTTAAGAAGCTCTGCTCCTTCCGTTACCGCAGGGATCTACTGAGACTCTCCTAT
GGTGAGGCCAAGAAAGCTGCCCGTGACTACGAGACGGCCAAGAACTACTTCAAAAAAGGCCTGAA
GGATATGGGCTATGGGAACTGGATTAGCAAACCCCAGGAGGAAAAGAACTTTTATCTCTGCCCAG
TATAGTATGCTCCAGTGACAGATGGATTAGGGTGTGTCATACTAGGGTGTGAGAGAGGTAGGTCG
TAGCATTCCTCATCACATGGTCAGGGGATTTTTTTTTCTCCTTTTTTTTTCTTTTTAAGCCATAA
TTGGTGATACTGAAAACTTTGGGTTCCCATTTATCCTGCTTTCTTTGGGATTGCTAGGCAAGGTC
TGGCCAGGCCCCCCTTTTTTCCCCCAAGTGAAGAGGCAGAAACCTAAGAAGTTATCTTTTCTTTC
TACCCAAAGCATACATAGTCACTGAGCACCTGCGGTCCATTTCCTCTTAAAAGTTTTGTTTTGAT
TTGTTTCCATTTCCTTTCCCTTTGTGTTTGCTACACTGACCTCTTGCGGTCTTGATTAGGTTTCA
GTCAACTCTGGATCATGTCAGGGACTGATAATTTCATTTGTGGATTACGCAGACCCCTCTACTTC
CCCTCTTTCCCTTCTGAGATTCTTTCCTTGTGATCTGAATGTCTCCTTTTCCCCCTCAGAGGGCA
AAGAGGTGAACATAAAGGATTTGGTGAAACATTTGTAAGGGTAGGAGTTGAAAACTGCAGTTCCC
AGTGCCACGGAAGTGTGATTGGAGCCTGCAGATAATGCCCAGCCATCCTCCCATCCTGCACTTTA
GCCAGCTGCAGGGCGGGCAAGGCAAGGAAAGCTGCTTCCCTGGAAGTGTATCACTTTCTCCGGCA
GCTGGGAAGTCTAGAACCAGCCAGACTGGGTTAAGGGAGCTGCTCAAGCAATAGCAGAGGTTTCA
CCCGGCAGGATGACACAGACCACTTCCCAGGGAGCACGGGCATGCCTTGGAATATTGCCAAGCTT
CCAGCTGCCTCTTCTCCTAAAGCATTCCTAGGAATATTTTCCCCGCCAATGCTGGGCGTACACCC
TAGCCAACGGGACAAATCCTAGAGGGTATAAAATCATCTCTGCTCAGATAATCATGACTTAGCAA
GAATAAGGGCAAAAAATCCTGTTGGCTTAACGTCACTGTTCCACCCGGTGTAATATCTCTCATGA
CAGTGACACCAAGGGAAGTTGACTAAGTCACATGTAAATTAGGAGTGTTTTAAAGAATGCCATAG
ATGTTGATTCTTAACTGCTACAGATAACCTGTAATTGAGCAGATTTAAAATTCAGGCATACTTTT
CCATTTATCCAAGTGCTTTCATTTTTCCAGATGGCTTCAGAAGTAGGCTCGTGGGCAGGGCGCAG
ACCTGATCTTTATAGGGTTGACATAGAAAGCAGTAGTTGTGGGTGAAAGGGCAGGTTGTCTTCAA
ACTCTGTGAGGTAGAATCCTTTGTCTATACCTCCATGAACATTGACTCGTGTGTTCAGAGCCTTT
GGCCTCTCTGTGGAGTCTGGCTCTCTGGCTCCTGTGCATTCTTTGAATAGTCACTCGTAAAAACT
GTCAGTGCTTGAAACTGTTTCCTTTACTCATGTTGAAGGGACTTTGTTGGCTTTTAGAGTGTTGG
TCATGACTCCAAGAGCAGAGCAGGGAAGAGCCCAAGCATAGACTTGGTGCCGTGGTGATGGCTGC
AGTCCAGTTTTGTGATGCTGCTTTTACGTGTCCCTCGATAACAGTCAGCTAGACACACTCAGGAG
GACTACTGAGGCTCTGCGACCTTCAGGAGCTGAGCCTGCCTCTCTCCTTTAGATGACAGACCTTC
ATCTGGGAACGTGCTGAGCCAGCACCCTCAGATGATTTCCCTCCAAACTGCTGACTAGGTCATCC
TCTGTCTGGTAGAGACATTCACATCTTTGCTTTTATTCTATGCTCTCTGTACTTTTGACCAAAAA
TTGACCAAAGTAAGAAAATGCAAGTTCTAAAAATAGACTAAGGATGCCTTTGCAGAACACCAAAG
CATCCCAAGGAACTGGTAGGGAAGTGGCGCCTGTCTCCTGGAGTGGAAGAGGCCTGCTCCCTGGC
TCTGGGTCTGCTGGGGGCACAGTAAATCAGTCTTGGCACCCACATCCAGGGCAGAGAGGTCTGTG
GTTCTCAGCATCAGAAGGCAGCGCAGCCCCTCTCCTCTTCAGGCTACAGGGTTGTCACCTGCTGA
GTCCTCAGGTTGTTTGGCCTCTCTGGTCCATCTTGGGCATTAGGTTCTCCAGCAGAGCTCTGGCC
AGCTGCCTCTTCTTTAACTGGGAACACAGGCTCTCACAAGATCAGAACCCCCACTCACCCCCAAG
ATCTTATCTAGCAAGCCTGTAGTATTCAGTTTCTGTTGTAGGAAGAGAGCGAGGCATCCCTGAAT
TCCACGCATCTGCTGGAAACGAGCCGTGTCAGATCGCACATCCCTGCGCCCCCATGCCCCTCTGA
GTCACACAGGACAGAGGAGGCAGAGCTTCTGCCCACTGTTATCTTCACTTTCTTTGTCCAGTCTT
TTGTTTTTAATAAGCAGTGACCCTCCCTACTCTTCTTTTTAATGATTTTTGTAGTTGATTTGTCT
GAACTGTGGCTACTGTGCATTCCTTGAATAATCACTTGTAAAAATTGTCAGTGCTTGAAGCTGTT
TCCTTTACTCACATTGAAGGGACTTCGTTGGTTTTTTGGAGTCTTGGTTGTGACTCCAAGAGCAG
AGTGAGGAAGACCCCCAAGCATAGACTCGGGTACTGTGATGATGGCTGCAGTCCAGTTTTATGAT
TCTGCTTTTATGTGTCCCTTGATAACAGTGACTTAACAATATACATTCCTCATAAATAAAAAAAA
AACAAGAATCTGAATTCTTAGAAA
NP_001351974.1
SEQ ID NO: 14
MSPICDQTIDSRLKGYSLSGYYTHPFQGYEHRQLRYQQPGPGSSPSSFLLKQIEFLKGQLPEAPV
IGKQTPSLPPSLPGLRPRFPVLLASSTRGRQVDIRGVPRGVHLRSQGLQRGFQHPSPRGRSLPQR
GVDCLSSHFQELSIYQDQEQRILKFLEELGEGKATTAHDLSGKLGTPKKEINRVLYSLAKKGKLQ
KEAGTPPLWKIAVSTQAWNQHSGVVRPDGHSQGAPNSDPSLEPEDRNSTSVSEDLLEPFIAVSAQ
AWNQHSGVVRPDSHSQGSPNSDPGLEPEDSNSTSALEDPLEFLDMAEIKEKICDYLFNVSDSSAL
NLAKNIGLTKARDINAVLIDMERQGDVYRQGTTPPIWHLTDKKRERMQIKRNTNSVPETAPAAIP
ETKRNAEFLTCNIPTSNASNNMVTTEKVENGQEPVIKLENRQEARPEPARLKPPVHYNGPSKAGY
VDFENGQWATDDIPDDLNSIRAAPGEFRAIMEMPSFYSHGLPRCSPYKKLTECQLKNPISGLLEY
AQFASQTCEFNMIEQSGPPHEPRFKFQVVINGREFPPAEAGSKKVAKQDAAMKAMTILLEEAKAK
DSGKSEESSHYSTEKESEKTAESQTPTPSATSFFSGKSPVTTLLECMHKLGNSCEFRLLSKEGPA
HEPKFQYCVAVGAQTFPSVSAPSKKVAKQMAAEEAMKALHGEATNSMASDNQPEGMISESLDNLE
SMMPNKVRKIGELVRYLNTNPVGGLLEYARSHGFAAEFKLVDQSGPPHEPKFVYQAKVGGRWFPA
VCAHSKKQGKQEAADAALRVLIGENEKAERMGFTEVTPVTGASLRRTMLLLSRSPEAQPKTLPLT
GSTFHDQIAMLSHRCENTLTNSFQPSLLGRKILAAIIMKKDSEDMGVVVSLGTGNRCVKGDSLSL
KGETVNDCHAEIISRRGFIRFLYSELMKYNSQTAKDSIFEPAKGGEKLQIKKTVSFHLYISTAPC
GDGALFDKSCSDRAMESTESRHYPVFENPKQGKLRTKVENGEGTIPVESSDIVPTWDGIRLGERL
RTMSCSDKILRWNVLGLQGALLTHFLQPIYLKSVTLGYLFSQGHLTRAICCRVTRDGSAFEDGLR
HPFIVNHPKVGRVSIYDSKRQSGKTKETSVNWCLADGYDLEILDGTRGTVDGPRNELSRVSKKNI
FLLFKKLCSFRYRRDLLRLSYGEAKKAARDYETAKNYFKKGLKDMGYGNWISKPQEEKNFYLCPV
NM_001365049.1
SEQ ID NO: 15
GAACCGGAGCCATCTTGGGCCCGGCGCGCAGACCCGCGGAGTTTCCCGTGCCGACGCCCCGGGGC
CACTTCCAGTGCGGAGTAGCGGAGGCGTGGGGGCCTCGAGGGGCTGGCGCGGCCCAGCGGTCGGG
CCAGGGTCGTGCCGCCGGCGGGTCGGGCCGGGCAATGCCTCGCGGGCGCAATGAATCCGCGGCAG
GGGTATTCCCTCAGCGGATACTACACCCATCCATTTCAAGGCTATGAGCACAGACAGCTCAGGTA
CCAGCAGCCTGGGCCAGGATCTTCCCCCAGTAGTTTCCTGCTTAAGCAAATAGAATTTCTCAAGG
GGCAGCTCCCAGAAGCACCGGTGATTGGAAAGCAGACACCGTCACTGCCACCTTCCCTCCCAGGA
CTCCGGCCAAGGTTTCCAGTACTACTTGCCTCCAGTACCAGAGGCAGGAACTGAGTATCTACCAA
GATCAGGAACAAAGGATCTTAAAGTTCCTGGAAGAGCTTGGGGAAGGGAAGGCCACCACAGCACA
TGATCTGTCTGGGAAACTTGGGACTCCGAAGAAAGAAATCAATCGAGTTTTATACTCCCTGGCAA
AGAAGGGCAAGCTACAGAAAGAGGCAGGAACACCCCCTTTGTGGAAAATCGCGGTCTCCACTCAG
GCTTGGAACCAGCACAGCGGAGTGGTAAGACCAGACGGTCATAGCCAAGGAGCCCCAAACTCAGA
CCCGAGTTTGGAACCGGAAGACAGAAACTCCACATCTGTCTCAGAAGATCTTCTTGAGCCTTTTA
TTGCAGTCTCAGCTCAGGCTTGGAACCAGCACAGCGGAGTGGTAAGACCAGACAGTCATAGCCAA
GGATCCCCAAACTCAGACCCAGGTTTGGAACCTGAAGACAGCAACTCCACATCTGCCTTGGAAGA
TCCTCTTGAGTTTTTAGACATGGCCGAGATCAAGGAGAAAATCTGCGACTATCTCTTCAATGTGT
CTGACTCCTCTGCCCTGAATTTGGCTAAAAATATTGGCCTTACCAAGGCCCGAGATATAAATGCT
GTGCTAATTGACATGGAAAGGCAGGGGGATGTCTATAGACAAGGGACAACCCCTCCCATATGGCA
TTTGACAGACAAGAAGCGAGAGAGGATGCAAATCAAGAGAAATACGAACAGTGTTCCTGAAACCG
CTCCAGCTGCAATCCCTGAGACCAAAAGAAACGCAGAGTTCCTCACCTGTAATATACCCACATCA
AATGCCTCAAATAACATGGTAACCACAGAAAAAGTGGAGAATGGGCAGGAACCTGTCATAAAGTT
AGAAAACAGGCAAGAGGCCAGACCAGAACCAGCAAGACTGAAACCACCTGTTCATTACAATGGCC
CCTCAAAAGCAGGGTATGTTGACTTTGAAAATGGCCAGTGGGCCACAGATGACATCCCAGATGAC
TTGAATAGTATCCGCGCAGCACCAGGTGAGTTTCGAGCCATCATGGAGATGCCCTCCTTCTACAG
TCATGGCTTGCCACGGTGTTCACCCTACAAGAAACTGACAGAGTGCCAGCTGAAGAACCCCATCA
GCGGGCTGTTAGAATATGCCCAGTTCGCTAGTCAAACCTGTGAGTTCAACATGATAGAGCAGAGT
GGACCACCCCATGAACCTCGATTTAAATTCCAGGTTGTCATCAATGGCCGAGAGTTTCCCCCAGC
TGAAGCTGGAAGCAAGAAAGTGGCCAAGCAGGATGCAGCTATGAAAGCCATGACAATTCTGCTAG
AGGAAGCCAAAGCCAAGGACAGTGGAAAATCAGAAGAATCATCCCACTATTCCACAGAGAAAGAA
TCAGAGAAGACTGCAGAGTCCCAGACCCCCACCCCTTCAGCCACATCCTTCTTTTCTGGGAAGAG
CCCCGTCACCACACTGCTTGAGTGTATGCACAAATTGGGGAACTCCTGCGAATTCCGTCTCCTGT
CCAAAGAAGGCCCTGCCCATGAACCCAAGTTCCAATACTGTGTTGCAGTGGGAGCCCAAACTTTC
CCCAGTGTGAGTGCTCCCAGCAAGAAAGTGGCAAAGCAGATGGCCGCAGAGGAAGCCATGAAGGC
CCTGCATGGGGAGGCGACCAACTCCATGGCTTCTGATAACCAGCCTGAAGGTATGATCTCAGAGT
CACTTGATAACTTGGAATCCATGATGCCCAACAAGGTCAGGAAGATTGGCGAGCTCGTGAGATAC
CTGAACACCAACCCTGTGGGTGGCCTTTTGGAGTACGCCCGCTCCCATGGCTTTGCTGCTGAATT
CAAGTTGGTCGACCAGTCCGGACCTCCTCACGAGCCCAAGTTCGTTTACCAAGCAAAAGTTGGGG
GTCGCTGGTTCCCAGCCGTCTGCGCACACAGCAAGAAGCAAGGCAAGCAGGAAGCAGCAGATGCG
GCTCTCCGTGTCTTGATTGGGGAGAACGAGAAGGCAGAACGCATGGGTTTCACAGAGCTCCCTCT
CACTGGCAGCACCTTCCATGACCAGATAGCCATGCTGAGCCACCGGTGCTTCAACACTCTGACTA
ACAGCTTCCAGCCCTCCTTGCTCGGCCGCAAGATTCTGGCCGCCATCATTATGAAAAAAGACTCT
GAGGACATGGGTGTCGTCGTCAGCTTGGGAACAGGGAATCGCTGTGTGAAAGGAGATTCTCTCAG
CCTAAAAGGAGAAACTGTCAATGACTGCCATGCAGAAATAATCTCCCGGAGAGGCTTCATCAGGT
TTCTCTACAGTGAGTTAATGAAATACAACTCCCAGACTGCGAAGGATAGTATATTTGAACCTGCT
AAGGGAGGAGAAAAGCTCCAAATAAAAAAGACTGTGTCATTCCATCTGTATATCAGCACTGCTCC
GTGTGGAGATGGCGCCCTCTTTGACAAGTCCTGCAGCGACCGTGCTATGGAAAGCACAGAATCCC
GCCACTACCCTGTCTTCGAGAATCCCAAACAAGGAAAGCTCCGCACCAAGGTGGAGAACGGAGAA
GGCACAATCCCTGTGGAATCCAGTGACATTGTGCCTACGTGGGATGGCATTCGGCTCGGGGAGAG
ACTCCGTACCATGTCCTGTAGTGACAAAATCCTACGCTGGAACGTGCTGGGCCTGCAAGGGGCAC
TGTTGACCCACTTCCTGCAGCCCATTTATCTCAAATCTGTCACATTGGGTTACCTTTTCAGCCAA
GGGCATCTGACCCGTGCTATTTGCTGTCGTGTGACAAGAGATGGGAGTGCATTTGAGGATGGACT
ACGACATCCCTTTATTGTCAACCACCCCAAGGTTGGCAGAGTCAGCATATATGATTCCAAAAGGC
AATCCGGGAAGACTAAGGAGACAAGCGTCAACTGGTGTCTGGCTGATGGCTATGACCTGGAGATC
CTGGACGGTACCAGAGGCACTGTGGATGGGCCACGGAATGAATTGTCCCGGGTCTCCAAAAAGAA
CATTTTTCTTCTATTTAAGAAGCTCTGCTCCTTCCGTTACCGCAGGGATCTACTGAGACTCTCCT
ATGGTGAGGCCAAGAAAGCTGCCCGTGACTACGAGACGGCCAAGAACTACTTCAAAAAAGGCCTG
AAGGATATGGGCTATGGGAACTGGATTAGCAAACCCCAGGAGGAAAAGAACTTTTATCTCTGCCC
AGTATAGTATGCTCCAGTGACAGATGGATTAGGGTGTGTCATACTAGGGTGTGAGAGAGGTAGGT
CGTAGCATTCCTCATCACATGGTCAGGGGATTTTTTTTTCTCCTTTTTTTTTCTTTTTAAGCCAT
AATTGGTGATACTGAAAACTTTGGGTTCCCATTTATCCTGCTTTCTTTGGGATTGCTAGGCAAGG
TCTGGCCAGGCCCCCCTTTTTTCCCCCAAGTGAAGAGGCAGAAACCTAAGAAGTTATCTTTTCTT
TCTACCCAAAGCATACATAGTCACTGAGCACCTGCGGTCCATTTCCTCTTAAAAGTTTTGTTTTG
ATTTGTTTCCATTTCCTTTCCCTTTGTGTTTGCTACACTGACCTCTTGCGGTCTTGATTAGGTTT
CAGTCAACTCTGGATCATGTCAGGGACTGATAATTTCATTTGTGGATTACGCAGACCCCTCTACT
TCCCCTCTTTCCCTTCTGAGATTCTTTCCTTGTGATCTGAATGTCTCCTTTTCCCCCTCAGAGGG
CAAAGAGGTGAACATAAAGGATTTGGTGAAACATTTGTAAGGGTAGGAGTTGAAAACTGCAGTTC
CCAGTGCCACGGAAGTGTGATTGGAGCCTGCAGATAATGCCCAGCCATCCTCCCATCCTGCACTT
TAGCCAGCTGCAGGGCGGGCAAGGCAAGGAAAGCTGCTTCCCTGGAAGTGTATCACTTTCTCCGG
CAGCTGGGAAGTCTAGAACCAGCCAGACTGGGTTAAGGGAGCTGCTCAAGCAATAGCAGAGGTTT
CACCCGGCAGGATGACACAGACCACTTCCCAGGGAGCACGGGCATGCCTTGGAATATTGCCAAGC
TTCCAGCTGCCTCTTCTCCTAAAGCATTCCTAGGAATATTTTCCCCGCCAATGCTGGGCGTACAC
CCTAGCCAACGGGACAAATCCTAGAGGGTATAAAATCATCTCTGCTCAGATAATCATGACTTAGC
AAGAATAAGGGCAAAAAATCCTGTTGGCTTAACGTCACTGTTCCACCCGGTGTAATATCTCTCAT
GACAGTGACACCAAGGGAAGTTGACTAAGTCACATGTAAATTAGGAGTGTTTTAAAGAATGCCAT
AGATGTTGATTCTTAACTGCTACAGATAACCTGTAATTGAGCAGATTTAAAATTCAGGCATACTT
TTCCATTTATCCAAGTGCTTTCATTTTTCCAGATGGCTTCAGAAGTAGGCTCGTGGGCAGGGCGC
AGACCTGATCTTTATAGGGTTGACATAGAAAGCAGTAGTTGTGGGTGAAAGGGCAGGTTGTCTTC
AAACTCTGTGAGGTAGAATCCTTTGTCTATACCTCCATGAACATTGACTCGTGTGTTCAGAGCCT
TTGGCCTCTCTGTGGAGTCTGGCTCTCTGGCTCCTGTGCATTCTTTGAATAGTCACTCGTAAAAA
CTGTCAGTGCTTGAAACTGTTTCCTTTACTCATGTTGAAGGGACTTTGTTGGCTTTTAGAGTGTT
GGTCATGACTCCAAGAGCAGAGCAGGGAAGAGCCCAAGCATAGACTTGGTGCCGTGGTGATGGCT
GCAGTCCAGTTTTGTGATGCTGCTTTTACGTGTCCCTCGATAACAGTCAGCTAGACACACTCAGG
AGGACTACTGAGGCTCTGCGACCTTCAGGAGCTGAGCCTGCCTCTCTCCTTTAGATGACAGACCT
TCATCTGGGAACGTGCTGAGCCAGCACCCTCAGATGATTTCCCTCCAAACTGCTGACTAGGTCAT
CCTCTGTCTGGTAGAGACATTCACATCTTTGCTTTTATTCTATGCTCTCTGTACTTTTGACCAAA
AATTGACCAAAGTAAGAAAATGCAAGTTCTAAAAATAGACTAAGGATGCCTTTGCAGAACACCAA
AGCATCCCAAGGAACTGGTAGGGAAGTGGCGCCTGTCTCCTGGAGTGGAAGAGGCCTGCTCCCTG
GCTCTGGGTCTGCTGGGGGCACAGTAAATCAGTCTTGGCACCCACATCCAGGGCAGAGAGGTCTG
TGGTTCTCAGCATCAGAAGGCAGCGCAGCCCCTCTCCTCTTCAGGCTACAGGGTTGTCACCTGCT
GAGTCCTCAGGTTGTTTGGCCTCTCTGGTCCATCTTGGGCATTAGGTTCTCCAGCAGAGCTCTGG
CCAGCTGCCTCTTCTTTAACTGGGAACACAGGCTCTCACAAGATCAGAACCCCCACTCACCCCCA
AGATCTTATCTAGCAAGCCTGTAGTATTCAGTTTCTGTTGTAGGAAGAGAGCGAGGCATCCCTGA
ATTCCACGCATCTGCTGGAAACGAGCCGTGTCAGATCGCACATCCCTGCGCCCCCATGCCCCTCT
GAGTCACACAGGACAGAGGAGGCAGAGCTTCTGCCCACTGTTATCTTCACTTTCTTTGTCCAGTC
TTTTGTTTTTAATAAGCAGTGACCCTCCCTACTCTTCTTTTTAATGATTTTTGTAGTTGATTTGT
CTGAACTGTGGCTACTGTGCATTCCTTGAATAATCACTTGTAAAAATTGTCAGTGCTTGAAGCTG
TTTCCTTTACTCACATTGAAGGGACTTCGTTGGTTTTTTGGAGTCTTGGTTGTGACTCCAAGAGC
AGAGTGAGGAAGACCCCCAAGCATAGACTCGGGTACTGTGATGATGGCTGCAGTCCAGTTTTATG
ATTCTGCTTTTATGTGTCCCTTGATAACAGTGACTTAACAATATACATTCCTCATAAATAAAAAA
AAAACAAGAATCTGAATTCTTAGAAA
NP_001351978.1
SEQ ID NO: 16
MAEIKEKICDYLFNVSDSSALNLAKNIGLTKARDINAVLIDMERQGDVYRQGTTPPIWHLTDKKR
ERMQIKRNTNSVPETAPAAIPETKRNAEFLTCNIPTSNASNNMVTTEKVENGQEPVIKLENRQEA
RPEPARLKPPVHYNGPSKAGYVDFENGQWATDDIPDDLNSIRAAPGEFRAIMEMPSFYSHGLPRC
SPYKKLTECQLKNPISGLLEYAQFASQTCEFNMIEQSGPPHEPRFKFQVVINGREFPPAEAGSKK
VAKQDAAMKAMTILLEEAKAKDSGKSEESSHYSTEKESEKTAESQTPTPSATSFFSGKSPVTTLL
ECMHKLGNSCEFRLLSKEGPAHEPKFQYCVAVGAQTFPSVSAPSKKVAKQMAAEEAMKALHGEAT
NSMASDNQPEGMISESLDNLESMMPNKVRKIGELVRYLNTNPVGGLLEYARSHGFAAEFKLVDQS
GPPHEPKFVYQAKVGGRWFPAVCAHSKKQGKQEAADAALRVLIGENEKAERMGFTELPLTGSTFH
DQIAMLSHRCENTLTNSFQPSLLGRKILAAIIMKKDSEDMGVVVSLGTGNRCVKGDSLSLKGETV
NDCHAEIISRRGFIRFLYSELMKYNSQTAKDSIFEPAKGGEKLQIKKTVSFHLYISTAPCGDGAL
FDKSCSDRAMESTESRHYPVFENPKQGKLRTKVENGEGTIPVESSDIVPTWDGIRLGERLRTMSC
SDKILRWNVLGLQGALLTHFLQPIYLKSVTLGYLFSQGHLTRAICCRVTRDGSAFEDGLRHPFIV
NHPKVGRVSIYDSKRQSGKTKETSVNWCLADGYDLEILDGTRGTVDGPRNELSRVSKKNIFLLFK
KLCSFRYRRDLLRLSYGEAKKAARDYETAKNYFKKGLKDMGYGNWISKPQEEKNFYLCPV

### EXAMPLES

The invention is now described with reference to the Examples below. These are not limiting on the scope of the invention, and a person skilled in the art would appreciate that suitable equivalents could be used within the scope of the present invention. Thus, the Examples may be considered component parts of the invention, and the individual aspects described therein may be considered as disclosed independently, or in any combination.

### Example 1—Identification of ADAR1 Inhibition as Causing a BRCA1 Synthetic Lethal Effect Using a High-Throughput Genetic Screen

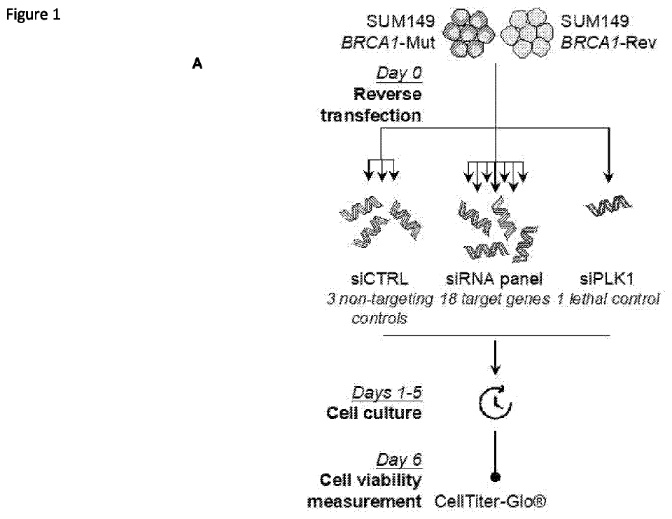

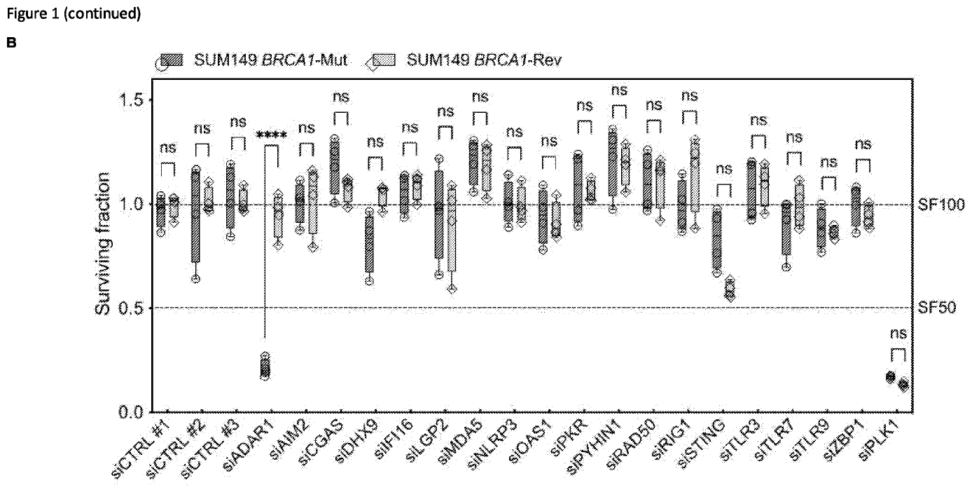

In order to understand which, if any, nucleic acid detecting pattern recognition receptors were critical for the fitness of BRCA1-mutant tumour cells, a high-throughput RNA interference (RNAi) screen was carried out to identify new BRCA1 synthetic lethal genes from amongst 18 genes encoding pattern recognition receptor proteins (FIG. 1A). In order to model BRCA1 deficiency, an isogenic pair of cell lines with/without BRCA1 function was used (Dréan et al. (2017) Mol. Cancer Ther. 16 (9): 2022-2034), namely SUM149 cells (referred to as SUM149 BRCA1-Mut in FIG. 1 and below) which have an endogenous BRCA1 loss of function mutation, BRCA1 c.2288delT, p.N723fsX13, and a daughter clone derived from SUM149 BRCA1-Mut, namely SUM149 B1.S* (referred to as SUM149 BRCA1-Rev in FIG. 1 and below), which has a functional BRCA1 gene due to a CRISPR-Cas9 engineered reversion mutation which restores homologous recombination function (Dréan et al. (2017) Mol. Cancer Ther. 16(9):2022-2034). Both cell lines were reverse transfected with a short interfering (si) RNA library (Dharmacon SMARTPools) arrayed in 96-well plates and then continuously cultured for six subsequent days, after which cell viability was assessed. By calculating the effect of each siRNA SMARTPool on cell viability as a surviving fraction (SF) compared to mock transfected cells we found that: (i) as expected, control, non-targeting, siRNA had little effect on either SUM149 BRCA1-Mut or SUM149 BRCA1-Rev cells; (ii) siRNA designed to target the essential gene PLK1 impaired the fitness of both SUM149 BRCA1-Mut and SUM149 BRCA1-Rev cells, implying the transfection in each cell line was highly efficient; and (iii) from the genes screened, siRNA targeting of ADAR1 (adenosine deaminase RNA specific, DSH; AGS6; G1P1; IFI4; P136; ADAR1; DRADA; DSRAD; IFI-4; K88DSRBP, ENSG00000160710) appeared to elicit synthetic lethal effects in SUM149 BRCA1-Mut but not SUM149 BRCA1-Rev cells, implying this gene could be synthetic lethal with BRCA1 (FIG. 1B).

### Example 2—BRCA1/ADAR1 Synthetic Lethality can be Elicited by CRISPR-Cas9 or siRNA Targeting of ADAR1 and Extends to Multiple Models of BRCA1 Deficiency

Two ADAR1 isoforms, ADAR1p150 (150 kDa) and ADAR1p110 (110 kDa), bind to and edit nucleic acids. Canonically, ADAR1p150 shuttles between the nucleus and cytoplasm, owing to the presence of a nuclear export signal at its amino terminus, whereas ADAR1p110 is largely retained in the nucleus (Patterson and Samuel (1995) Mol. Cell Biol. 15(10):5376-5388; Poulsen et al. (2001) Mol. Cell Biol. 21(22):7862-7871; Barraud et al. (2014) Proc. Natl. Acad. Sci. USA 111(18):E1852-E1861; Strehblow et al. (2002) Mol. Biol. Cell 13(11):3822-3835). Homodimer formation has been reported for both ADAR1p150 and ADAR1p110, and in the case of ADAR1p150, this is thought to be essential for efficient RNA editing catalytic activity (Chilibeck et al. (2006) J. Biol. Chem. 281(24):16530-16535; Cho et al. (2003) J. Biol. Chem. 278(19):17093-17102; Valente and Nishikura (2007) J. Biol. Chem. 282(22):16054-16061).

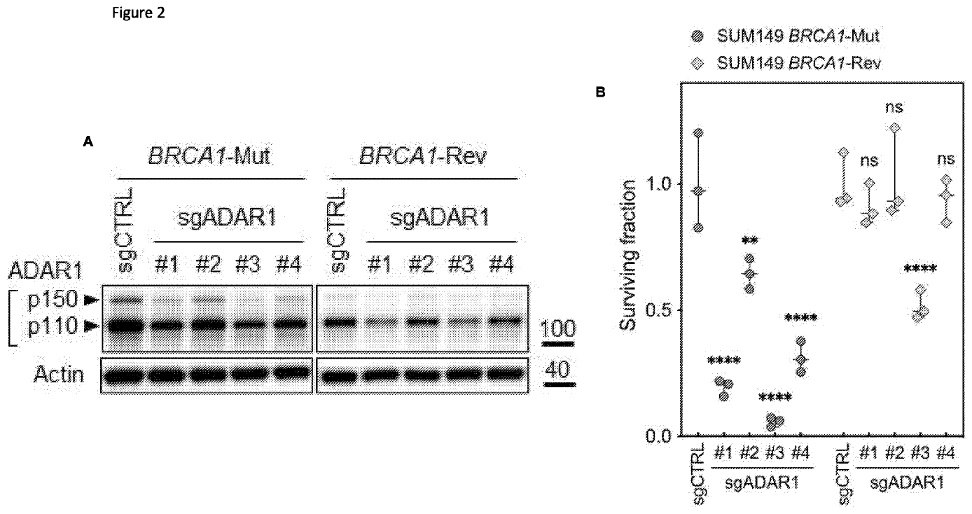

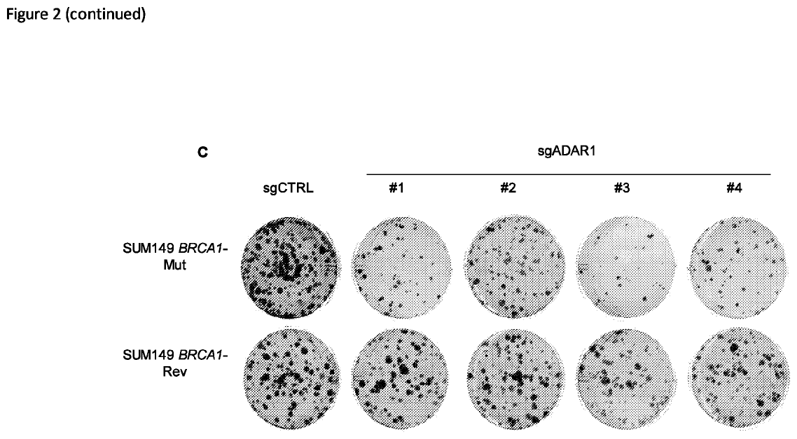

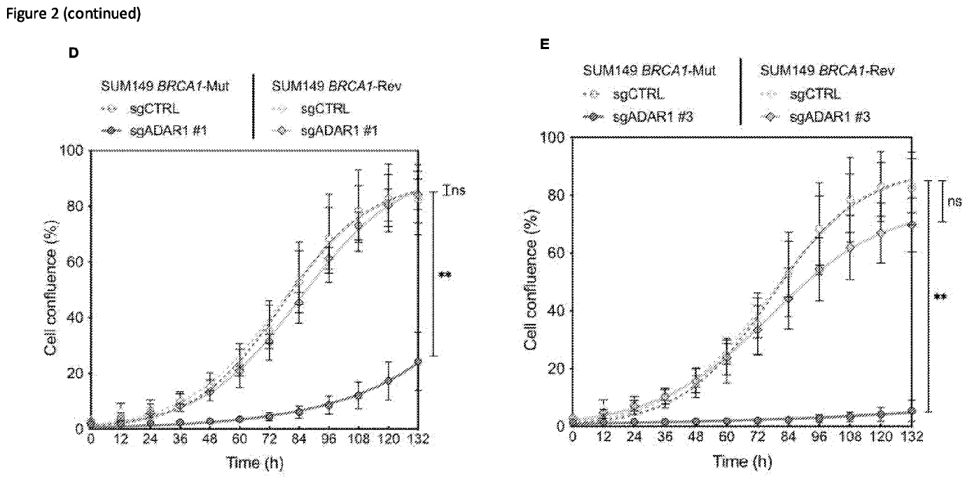

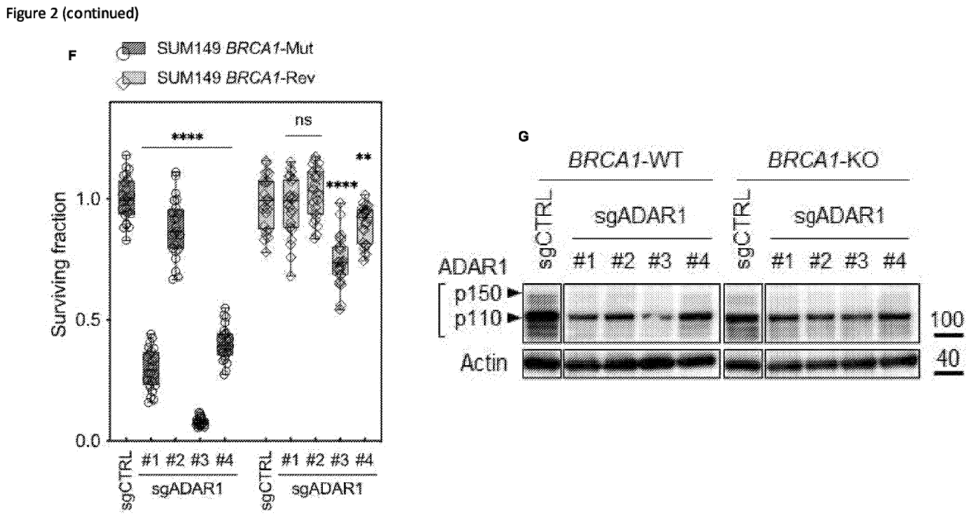

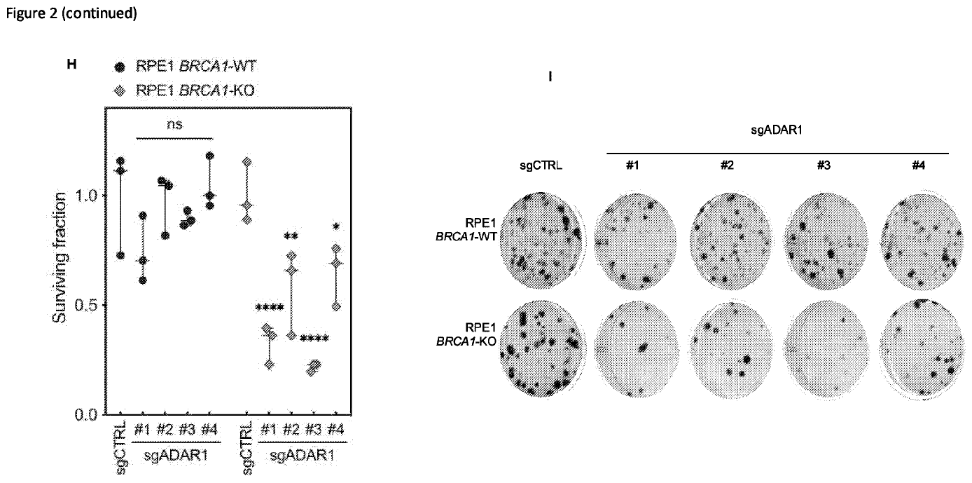

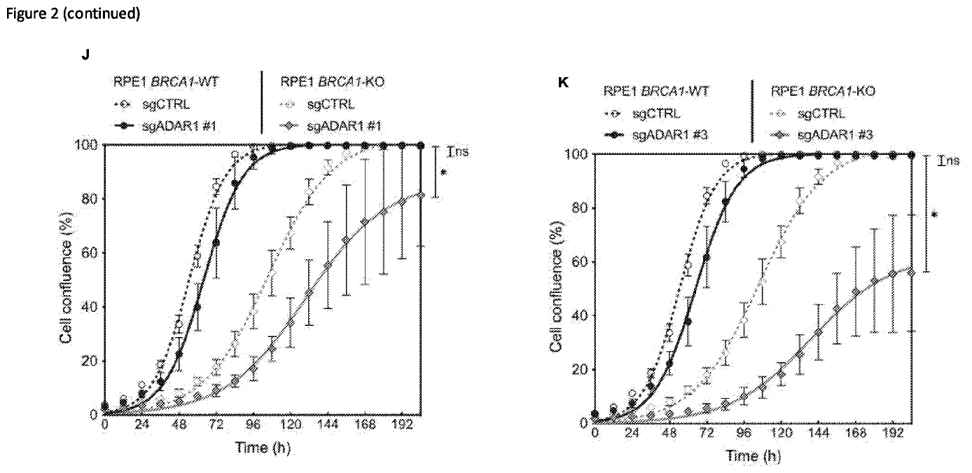

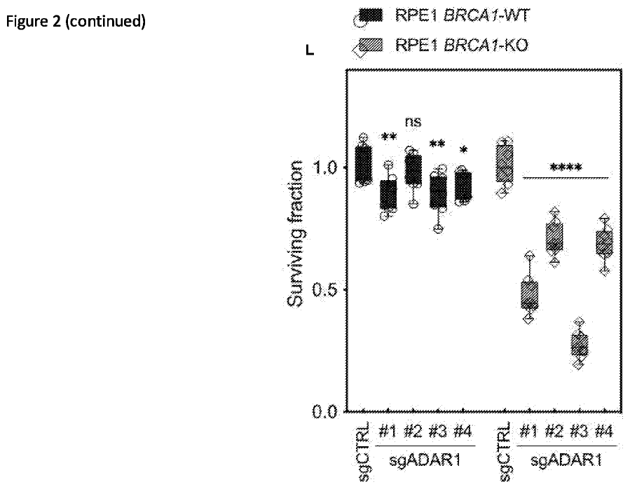

CRISPR-Cas9 editing of the ADAR1 gene was found to reduce levels of both ADAR1p110 and ADAR1p150 ADAR1 isoforms (FIG. 2A) and reduce clonogenic survival of SUM149 BRCA1-Mut cells compared to SUM149 BRCA1-Rev cells (FIG. 2B, C). These effects were achieved using multiple different ADAR1 guide RNAs (sgRNA, FIG. 2A-C), indicating that these were unlikely to be the result of off-target effects.

Using a different experimental assay, cell confluence was estimated by use of an Incucyte platform; in this setting, BRCA1/ADAR1 synthetic lethality was also observed with CRISPR-Cas9 editing of the ADAR1 gene (FIG. 2D-F). To eliminate the possibility that these effects were private to SUM149 cells and not a more generalisable effect in BRCA1-mutant cells, CRISPR-Cas9 editing of the ADAR1 gene was conducted in BRCA1-wildtype and BRCA1-mutant RPE1 cells (retinal pigment epithelial cells, shown as RPE1 BRCA1-WT and RPE1 BRCA1-KO cells in FIG. 2, respectively). As in SUM149 cells, ADAR1 sgRNA also reduced the total amount of both ADAR1 isoforms and elicited synthetic lethality in BRCA1-KO but not BRCA1-WT RPE1 cells (FIG. 2G-L).

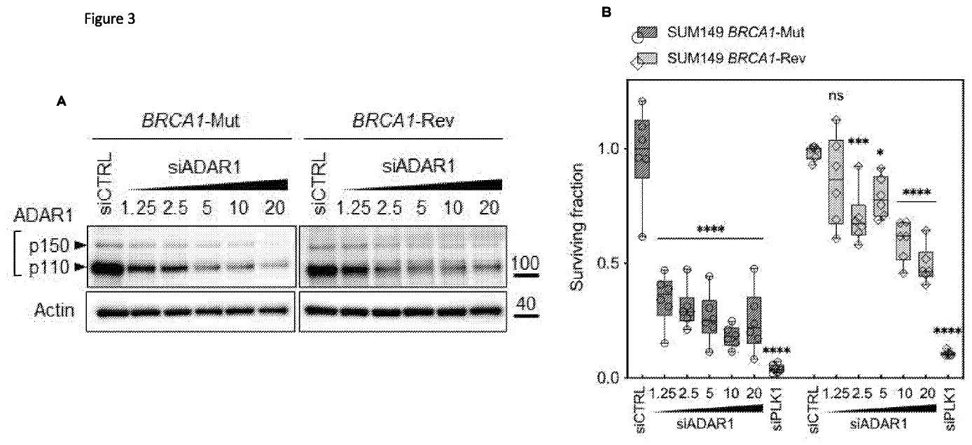

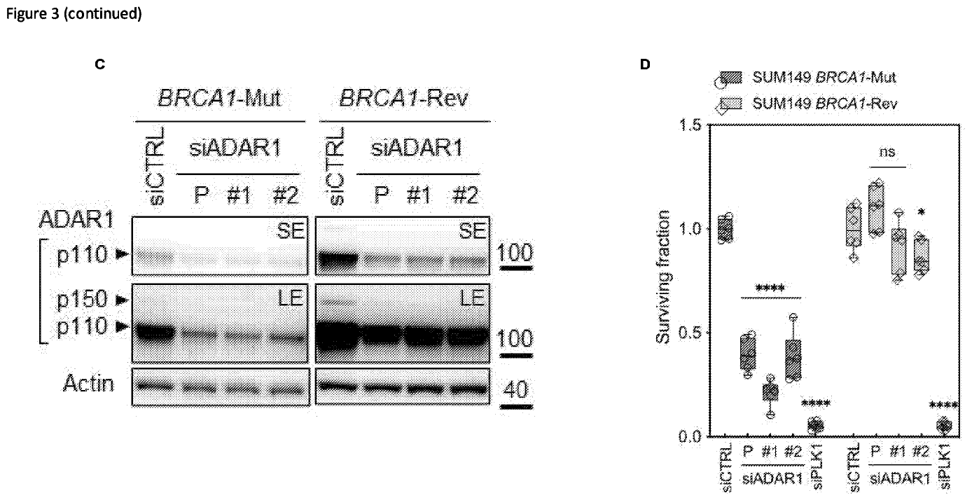

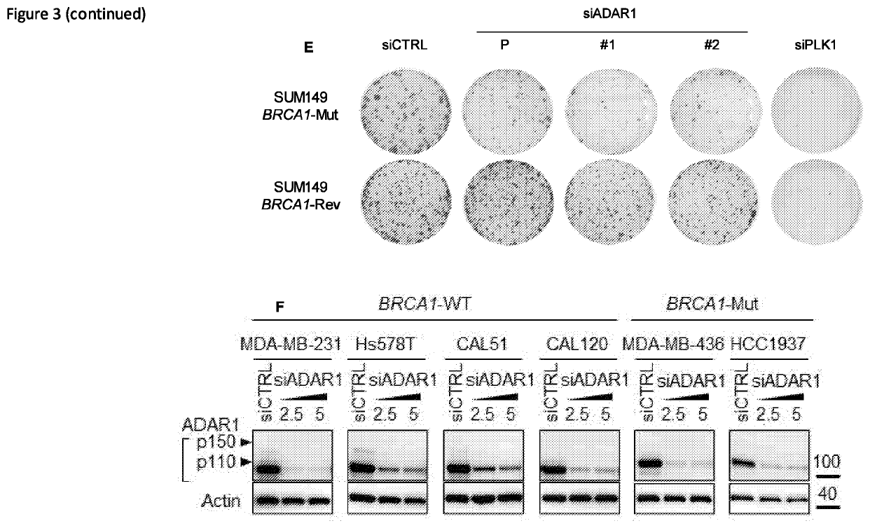

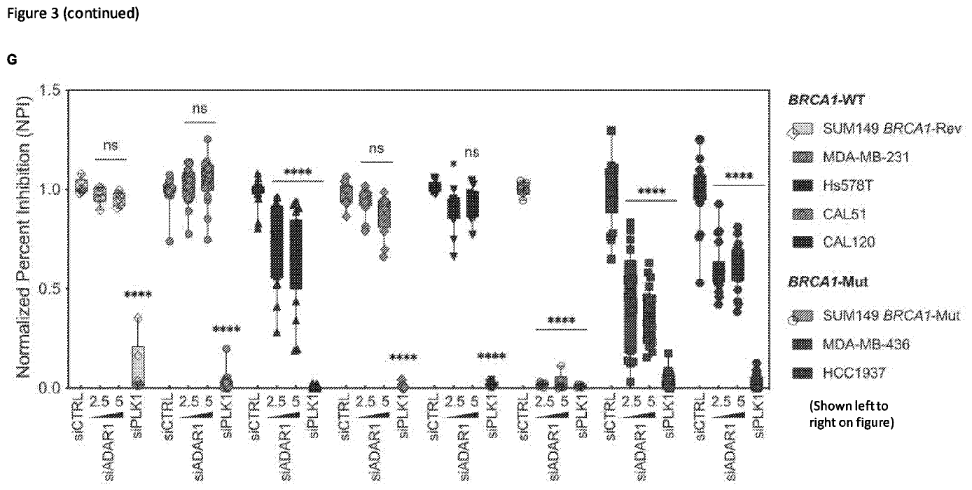

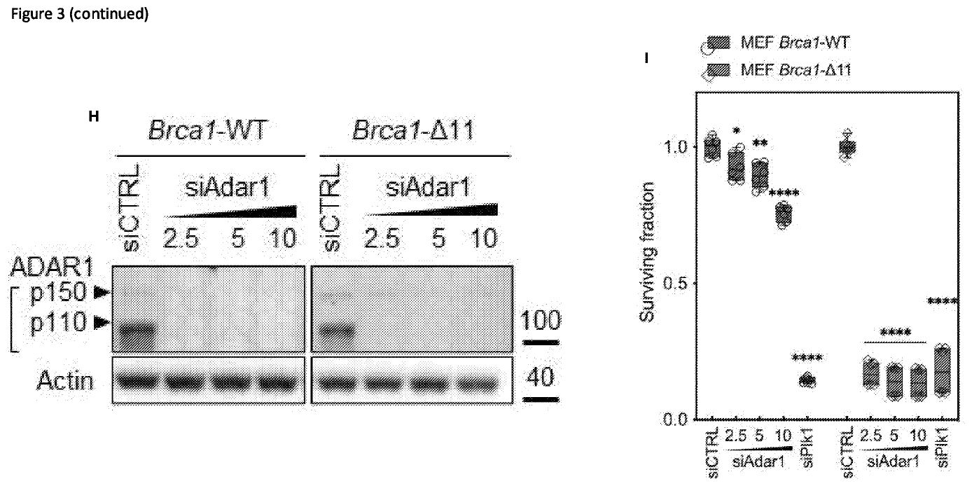

To confirm these effects were not private to CRISPR-Cas9 targeting of ADAR1, the effects of ADAR1-targeting siRNAs were also assessed (FIG. 3). The use of either an siRNA SMARTpool reagent (a mixture of four individual siRNAs targeting ADAR1 sequence) or individual ADAR1-targeting siRNAs almost completely silenced the expression of ADAR1 isoforms and elicited synthetic lethality in SUM149 BRCA1-Mut cells (FIG. 3A-E). These experiments indicated that the BRCA1/ADAR1 synthetic lethal effect could be elicited by RNA interference of ADAR1 (FIG. 3A-E) and extended to tumour cell lines with naturally occurring BRCA1 mutations such as the triple-negative breast tumour cell lines HCC1937 and MDA-MB-436 (FIG. 3F, G). The BRCA1/ADAR1 synthetic lethal effect was also noted to be conserved across the species barrier, as ADAR1 RNA interference also elicited synthetic lethality in Brca1-mutant mouse embryonic fibroblasts (shown as MEF Brca1-Δ11 in FIG. 3H-J).

### Example 3—CRISPR-Cas9 Targeting of ADAR1 Confers Synthetic Lethality to BRCA2-Mutant Cells

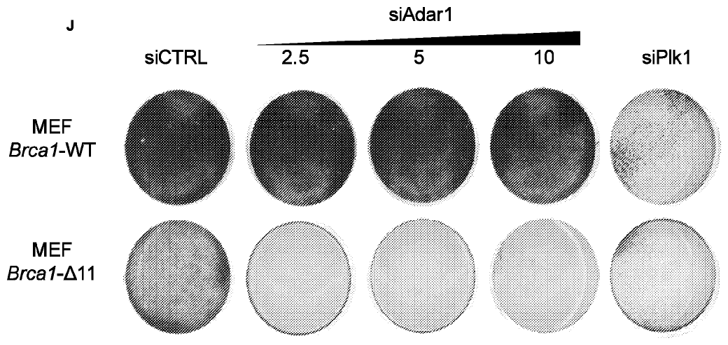

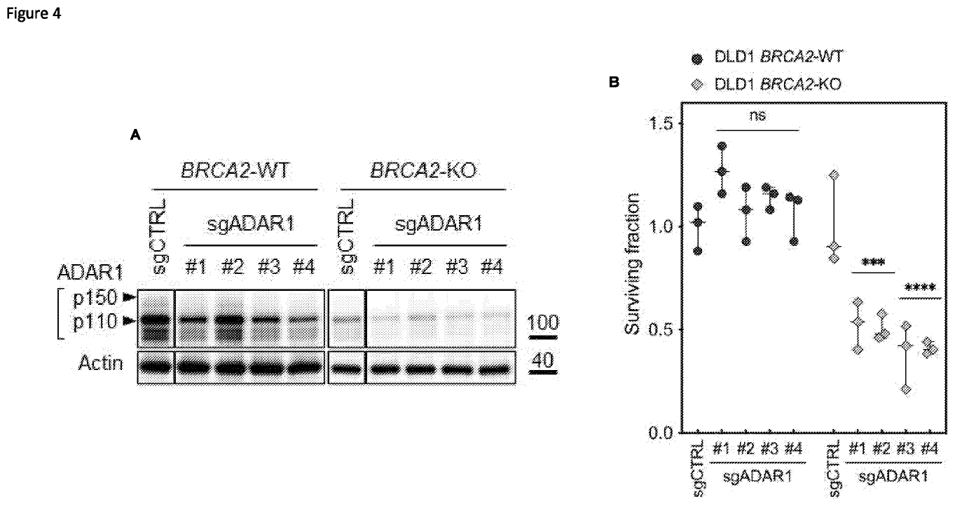

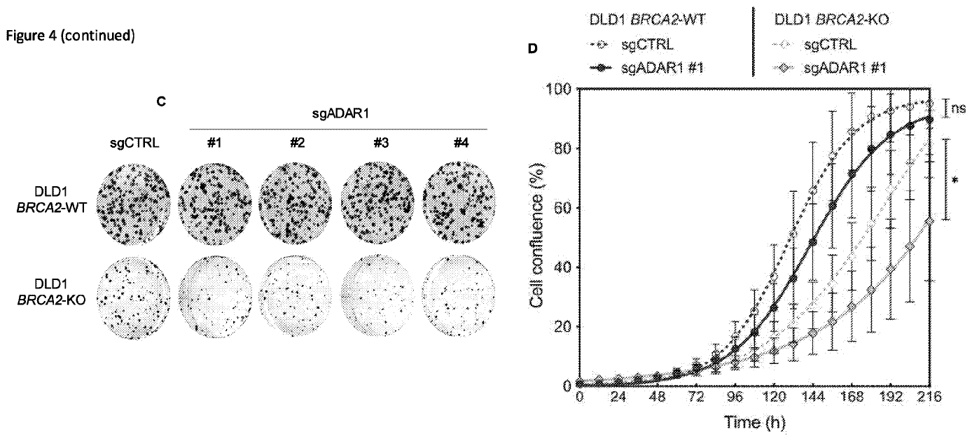

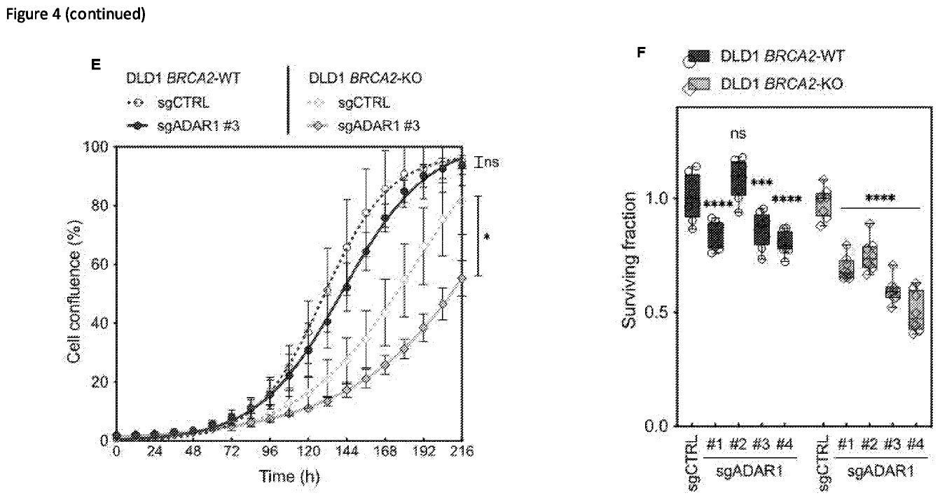

To assess whether ADAR1-dependent synthetic lethal effects extended beyond BRCA1-mutant cells to cells with other homologous recombination defects, CRISPR-Cas9 editing of the ADAR1 gene was repeated in BRCA2-wildtype and BRCA2-mutant DLD1 cells (colorectal adenocarcinoma cells, shown as DLD1 BRCA2-WT and DLD1 BRCA2-KO cells in FIG. 4, respectively). As in SUM149 and RPE1 cells, ADAR1 sgRNA reduced the total amount of ADAR1 isoforms and elicited synthetic lethality in BRCA2-KO but not BRCA2-WT DLD1 cells (FIG. 4A-F).

### Example 4—siRNA Targeting of BRCA1 or BRCA2 Elicits Reciprocal Synthetic Lethality in a Model of ADAR1 Deficiency

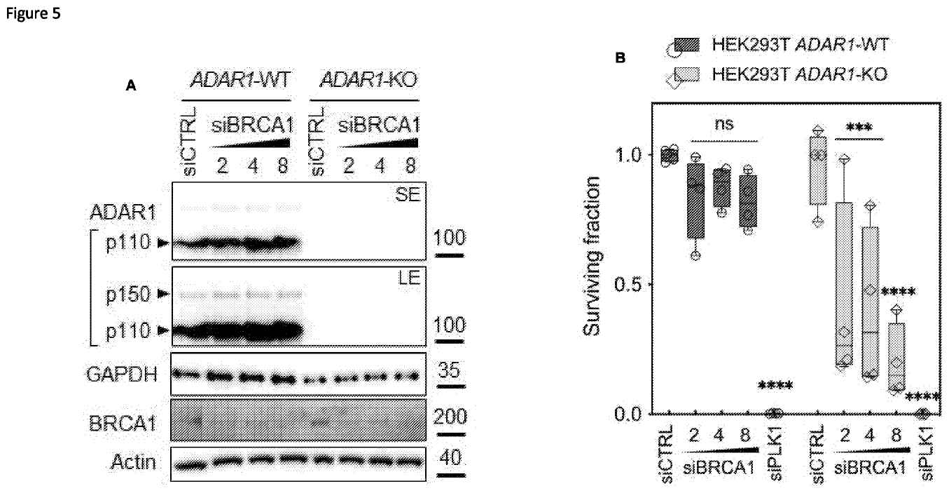

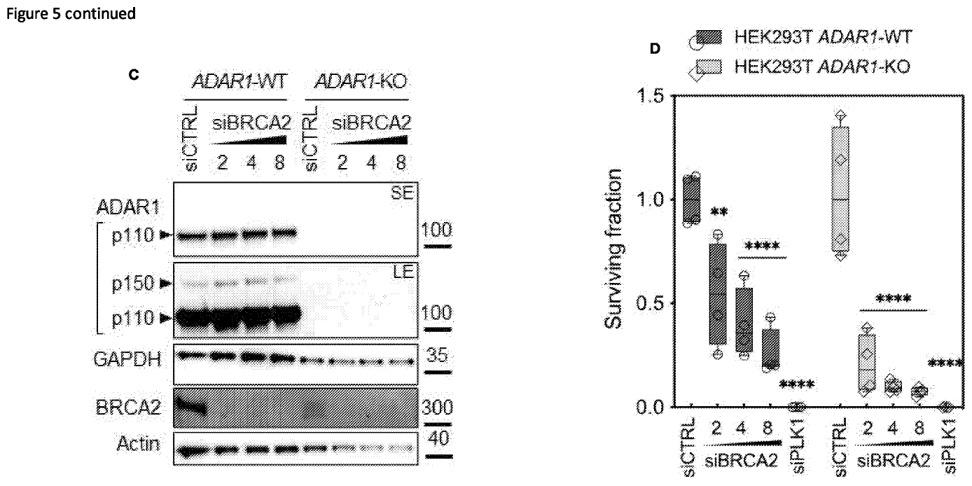

To confirm the genetic basis of BRCA/ADAR1 synthetic lethality, its reciprocity was tested by assessing the effects of BRCA1- or BRCA2-targeting siRNAs in a model of ADAR1 deficiency (Chung et al. (2018) Cell 172(4):811-824) consisting of isogenic ADAR1-wildtype and ADAR1-mutant HEK293T cells (human embryonic kidney cells, shown as HEK293T ADAR1-WT and HEK293T ADAR1-KO cells in FIG. 5, respectively). The BRCA/ADAR1 synthetic lethal effect was found to operate in such reciprocal setting, as BRCA1 or BRCA2 RNA interference elicited synthetic lethality in ADAR1-KO but not ADAR1-WT HEK293T cells (FIG. 5A-D).

### Example 5—BRCA1/ADAR1 Synthetic Lethality Operates in a Context of PARP Inhibitor Resistance

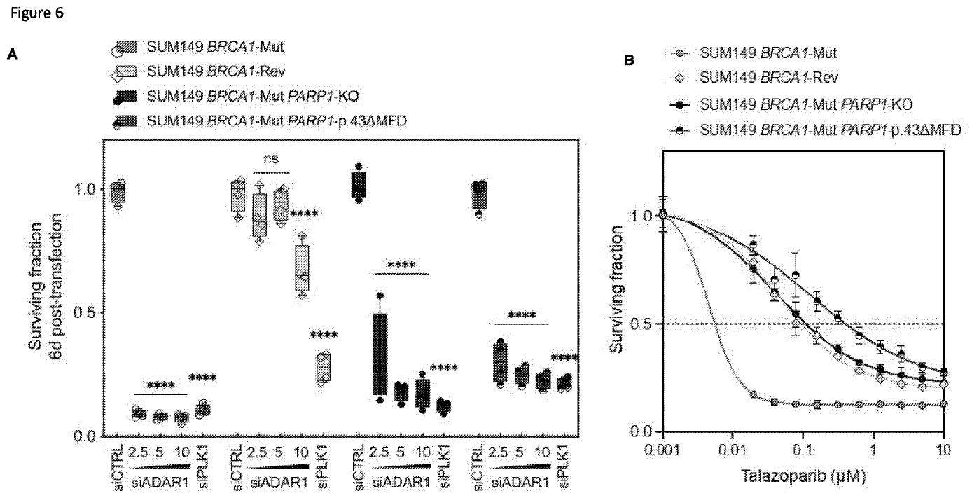

In order to model PARP inhibitor resistance, two daughter clones derived from the SUM149 BRCA1-Mut cell line were used (Pettitt et al. (2018) Nat. Commun. 9 (1): 1849; sensitivity to PARP inhibitor talazoparib shown in FIG. 6B): one SUM149 subclone with PARP inhibitor resistance caused by loss of PARP1 expression (shown as SUM149 BRCA1-Mut PARP1-KO in FIG. 6) and a second SUM149 subclone with PARP inhibitor resistance caused by loss of PARP1 DNA binding ability (shown as SUM149 BRCA1-Mut PARP1-p.43ΔMFD). ADAR1 RNA interference elicited synthetic lethality in PARP1-KO and PARP1-p.43ΔMFD BRCA1-Mut cells to the same extent as in parental SUM149 BRCA1-Mut cells, indicating that ADAR1-dependent synthetic lethal effects operated in a context of PARP inhibitor resistance (FIG. 6A).

### Example 6—Chemical Inhibition of ADAR1 Elicits BRCA1 and BRCA2 Synthetic Lethality

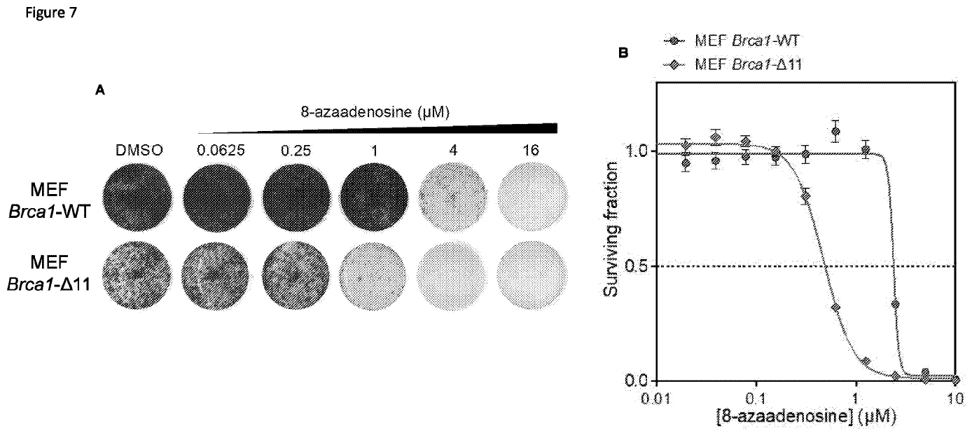

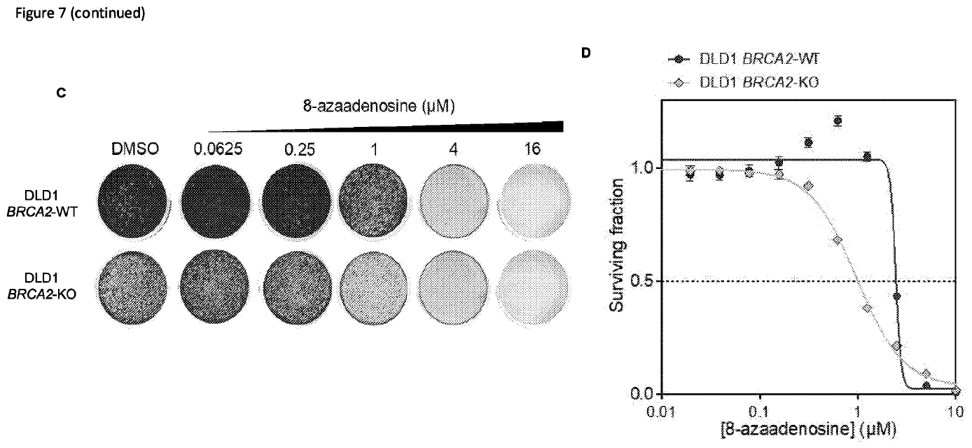

Using a toolbox small molecule inhibitor of ADAR1, 8-azaadenosine, it was demonstrated that chemical inhibition of ADAR1 also elicited BRCA1 synthetic lethality in Brca1-mutant MEFs (shown as MEF Brca1-Δ11 in FIG. 7A, B) and BRCA2 synthetic lethality in BRCA2 mutant DLD1 cells (shown as DLD1 BRCA2-KO in FIG. 7C, D).

### Example 7—siRNA Targeting of ADAR1 Causes an Accumulation of DNA Damage and Selective Genomic Instability in BRCA1-Mutant and BRCA2-Mutant Cells

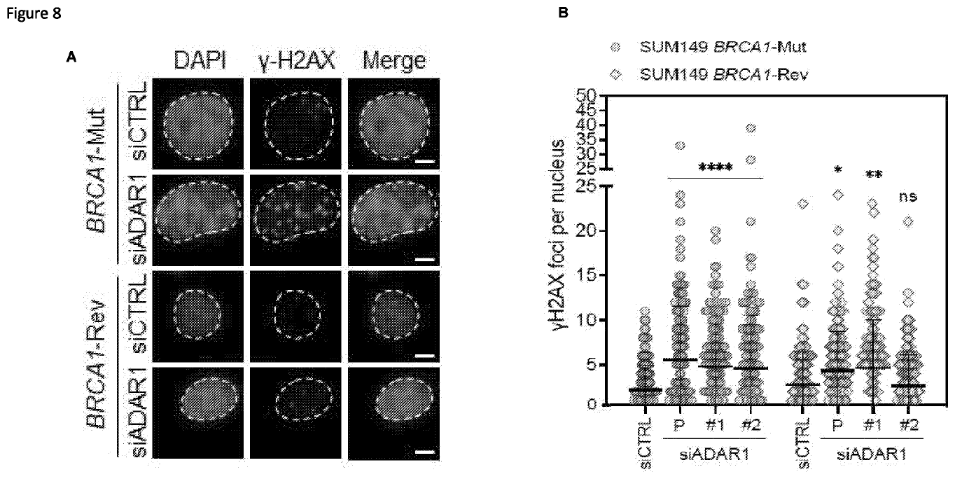

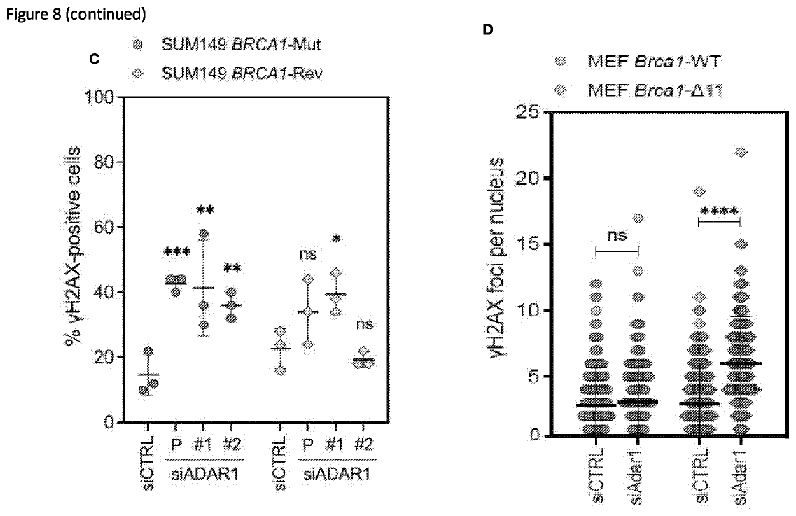

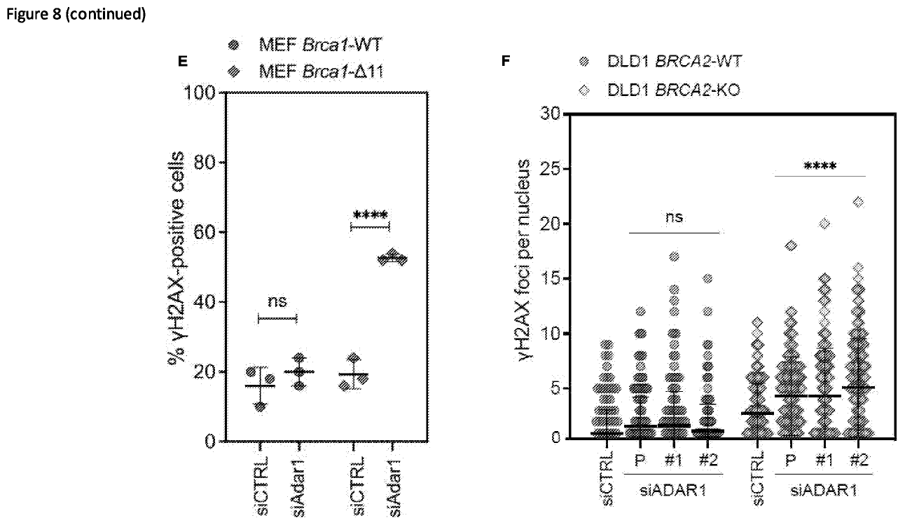

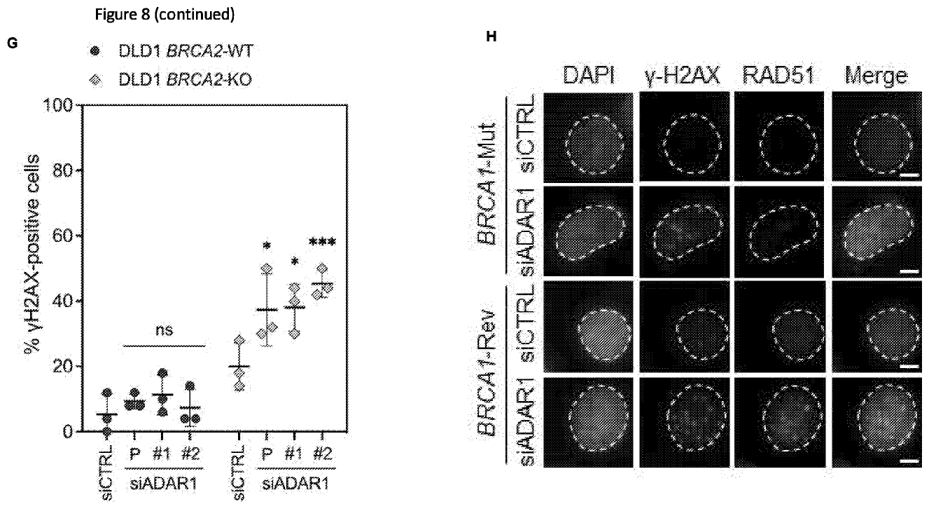

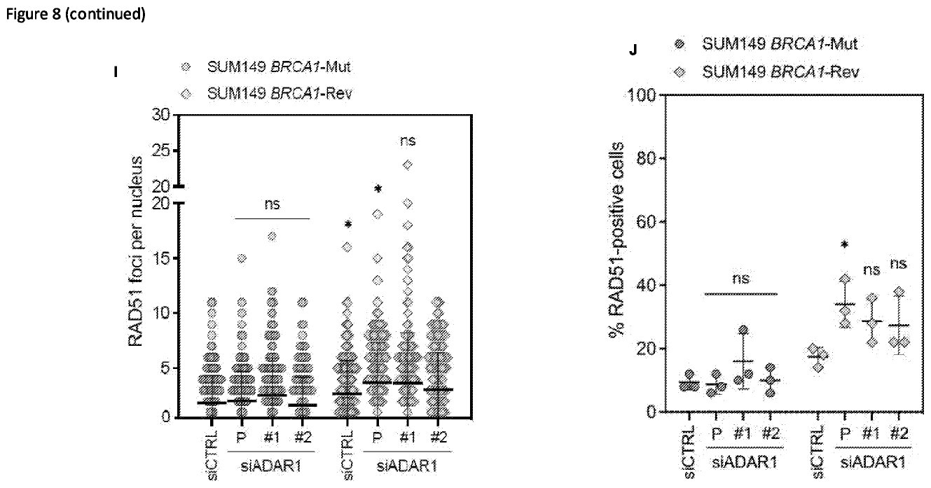

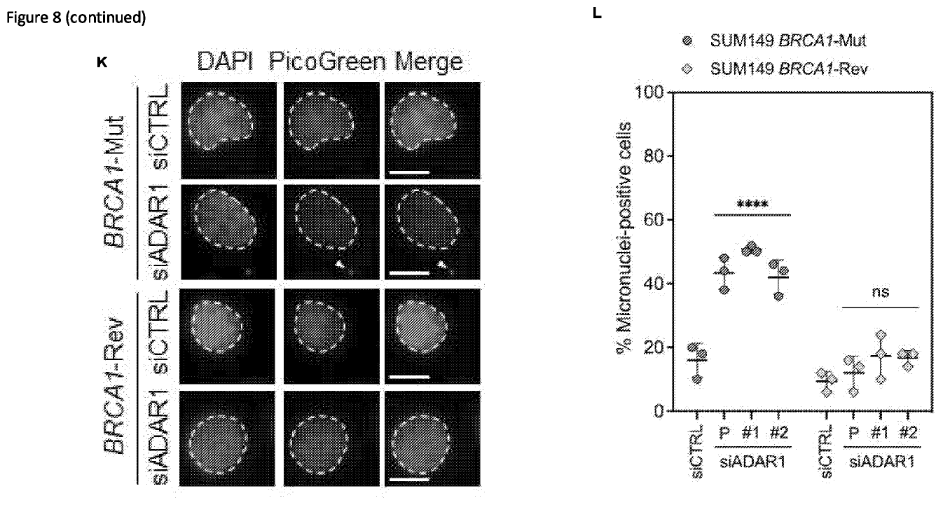

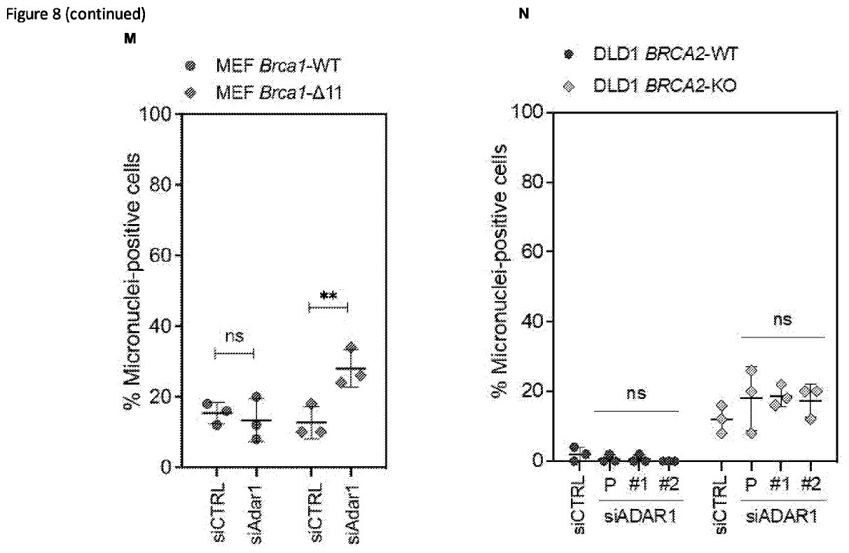

To investigate the potential causes of BRCA/ADAR1 synthetic lethality, the formation of γ-H2AX foci, RAD51 foci and micronuclei, biomarkers of DNA damage, homologous recombination, and genomic instability, respectively, was monitored in BRCA1-Mut and BRCA1-Rev SUM149 cells, Brca1-wildtype and Brca1-mutant MEFs (shown as MEF Brca1-WT and MEF Brca1-Δ11 in FIG. 8, respectively), and BRCA2-WT and BRCA2-KO DLD1 cells (shown as DLD1 BRCA2-WT and DLD1 BRCA2-KO cells in FIG. 8, respectively).

These experiments indicated that in SUM149 cells, siRNA silencing of ADAR1 increased the number of γ-H2AX foci to similar levels in BRCA1-Mut and BRCA1-Rev cells (FIG. 8A-C) whereas in MEFs and DLD1 cells, siRNA silencing of ADAR1 caused a selective accumulation of γ-H2AX foci in Brca1-Δ11 and BRCA2-KO cells (FIG. 8D-G). It was further noted that this accumulation of DNA damage caused by ADAR1 silencing was associated with: (i) increased numbers of RAD51 foci in SUM149 BRCA1-Rev cells, suggesting an ongoing DNA damage repair through homologous recombination (FIG. 8H-J); and (ii) increased numbers of micronuclei in BRCA1-Mut SUM149 cells, Brca1-Δ11 MEFs and BRCA2-KO DLD1 cells, suggesting selectively enhanced levels of genomic instability in BRCA-mutant cells (FIG. 8K-N).

### Example 8—siRNA Targeting of ADAR1 Increases Replication Stress and R-Loop Burden in BRCA1-Mutant Cancer Cells, Resulting in Activation of the Replication Stress Response and Apoptosis

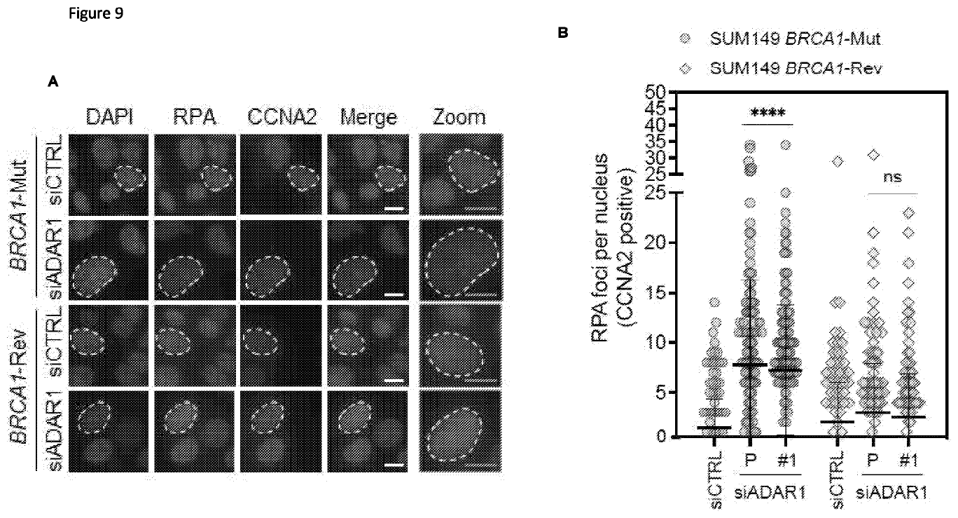

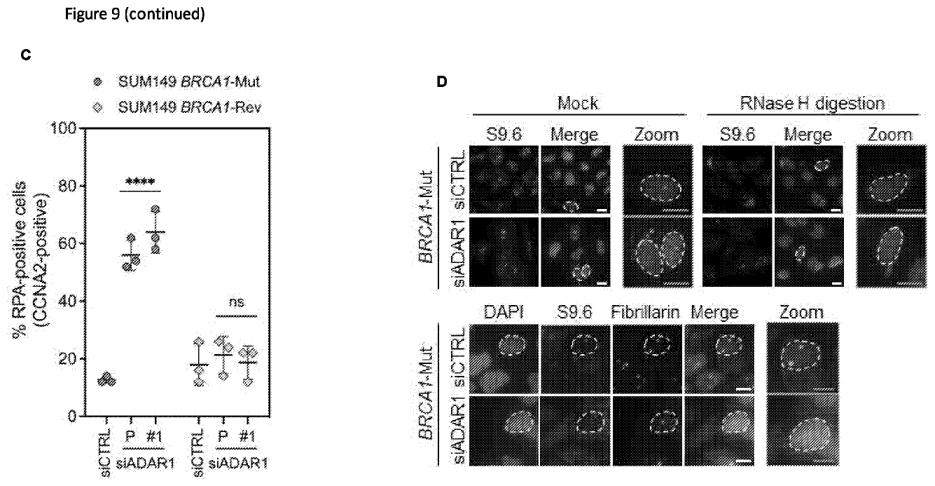

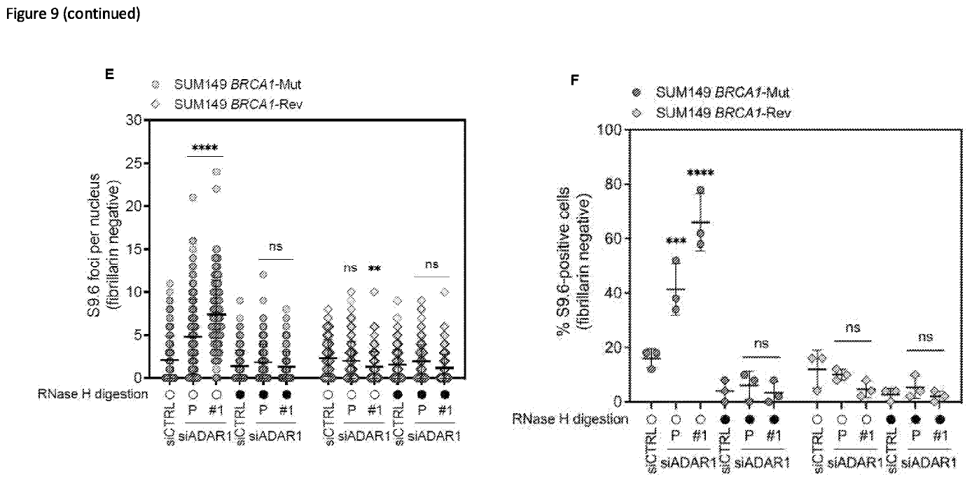

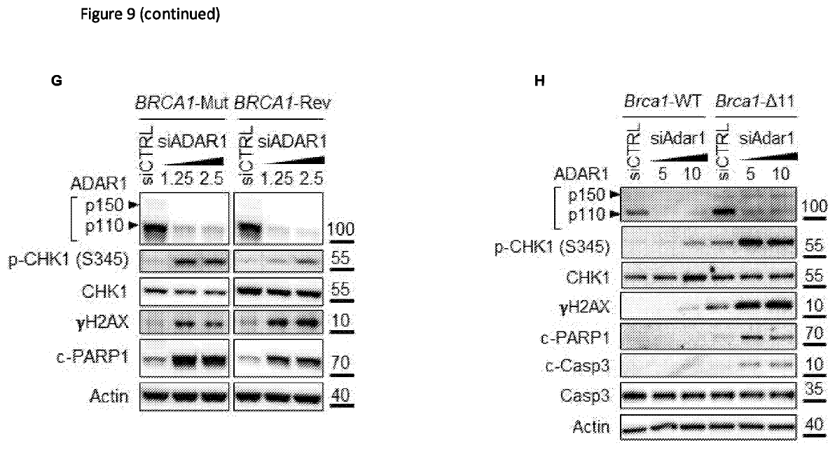

To investigate the potential causes of ADAR1-dependent DNA damage and genomic instability in BRCA-mutant cells, the formation of RPA foci in S-phase-positive cells, a surrogate biomarker of replication stress, was monitored in BRCA1-Mut and BRCA1-Rev SUM149 cells. siRNA silencing of ADAR1 was noted to increase the number of RPA foci in BRCA1-Mut but not BRCA1-Rev cells (FIG. 9A-C), suggesting selectively enhanced replication stress in BRCA-mutant cells.

ADAR1 isoforms and their paralog ADAR2 edit RNA: DNA hybrids (including genomic R-loops) thereby facilitating their resolution by RNA endonucleases such as RNase H2 and RNA: DNA helicases such as SETX, DHX9 or DDX21 (Shiromoto et al. (2021) Nat. Commun. 12(1):1654; Jimeno et al. (2021) Nat. Commun. 12(1):5512; Tang et al. (2022) Nature 606(7916):930-936; Zhang et al. (2023) Nuc. Acids Res. 51(21):11668-11687). The loss of ADAR1 has been reported to increase R-loop burden and R-loop-associated genomic instability (Shiromoto et al. (2021) Nat. Commun. 12(1):1654; Zhang et al. (2023) Nuc. Acids Res. 51(21):11668-11687), suggesting that ADAR1 safeguards the genome against the threats posed by R-loop accumulation by promoting their clearance.

To assess whether BRCA/ADAR1 synthetic lethality was associated with altered R-loop levels, the formation of genomic R-loops was monitored in BRCA1-Mut and BRCA1-Rev SUM149 cells by use of the RNA: DNA hybrid-specific S9.6 antibody. siRNA silencing of ADAR1 was found to increase the number of nuclear S9.6 foci in BRCA1-Mut but not BRCA1-Rev cells (FIG. 9D-E), indicating a selectively enhanced R-loop burden in BRCA-mutant cells. It was further noted that this accumulation of R-loop caused by ADAR1 silencing was associated with: (i) an increased phosphorylation of CHK1 (p-CHK1) and (ii) increased levels of cleaved-PARP1 (c-PARP1) and cleaved-caspase 3 (c-Casp3) in BRCA1-Mut SUM149 cells and Brca1-Δ11 MEFs, suggesting a selective activation of the replication stress checkpoint and apoptosis, respectively, in BRCA-mutant cells (FIG. 9G, H).

### Example 9—BRCA1/ADAR1 Synthetic Lethality is Reversed by Overexpression of the R-Loop-Degrading Enzyme RNase H1

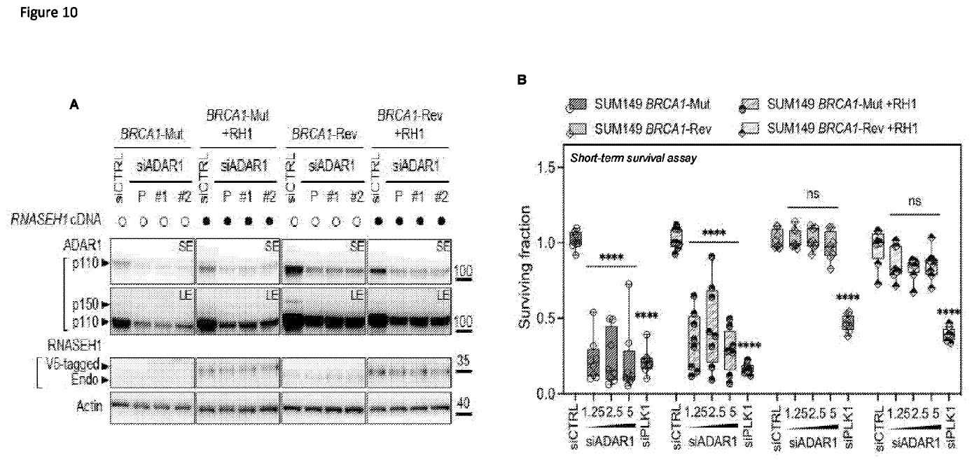

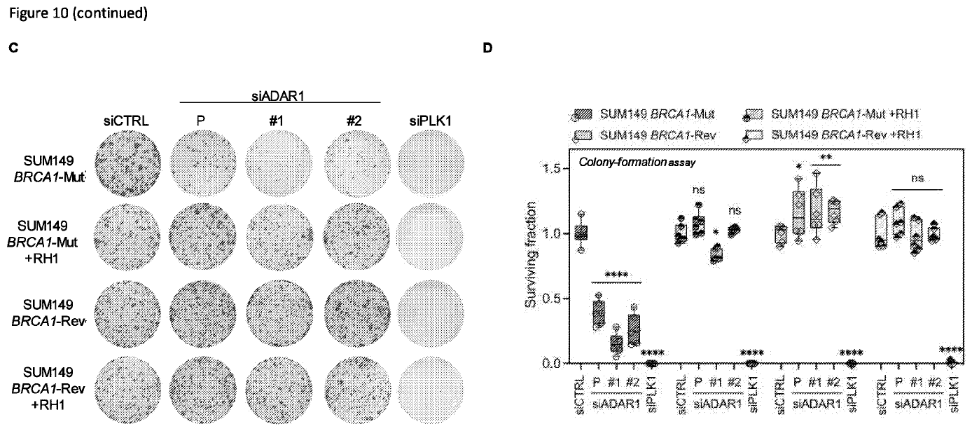

To determine whether R-loop levels modulate BRCA/ADAR1 synthetic lethality, the R-loop-degrading enzyme RNase H1 was overexpressed in BRCA1-Mut and BRCA1-Rev SUM149 cells (FIG. 10A) and the effects of ADAR1-targeting siRNAs were assessed. In this experiment, exogenous overexpression of RNase H1 reduced the synthetic lethal effects of ADAR1 siRNA silencing in SUM149 BRCA1-Mut cells as measured by cell viability (FIG. 10B) and clonogenic survival (FIG. 10C, D), identifying R-loops as a potential cause of BRCA/ADAR1 synthetic lethality.

### Example 10—BRCA1/ADAR1 Synthetic Lethality Requires RNA Sensors, and is Abrogated by Pharmacological Inhibition of the JAK/STAT Pathway

ADAR1-mediated RNA editing has been reported to prevent the detection of endogenous RNA species by pattern recognition receptors, notably RNA sensors such as RIG-1, MDA5, LGP2 and PKR (Mannion et al. (2014) Cell Rep. 9(4):1482-1494; Liddicoat et al. (2015) Science 349(6252):1115-1120; Pestal et al. (2015) Immunity 43(5):933-944; Chung et al. (2018) Cell 172(4):811-824; de Reuver et al. (2021) Cell Rep. 36(6):109500; Stok et al. (2022) EMBO J. 41(6):e109760; Maurano et al. (2021) Immunity 54(9):1948-1960; Li et al. (2010) Virology 396(2):316-322).

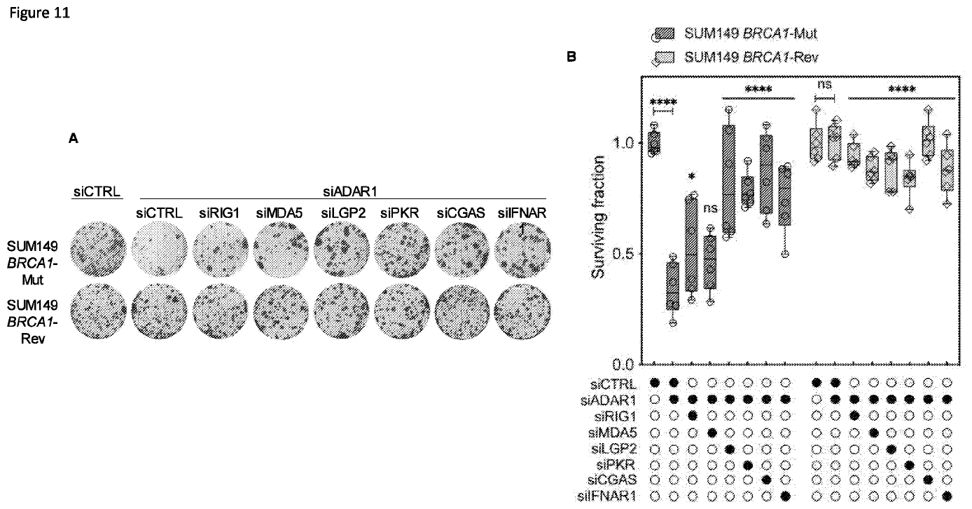

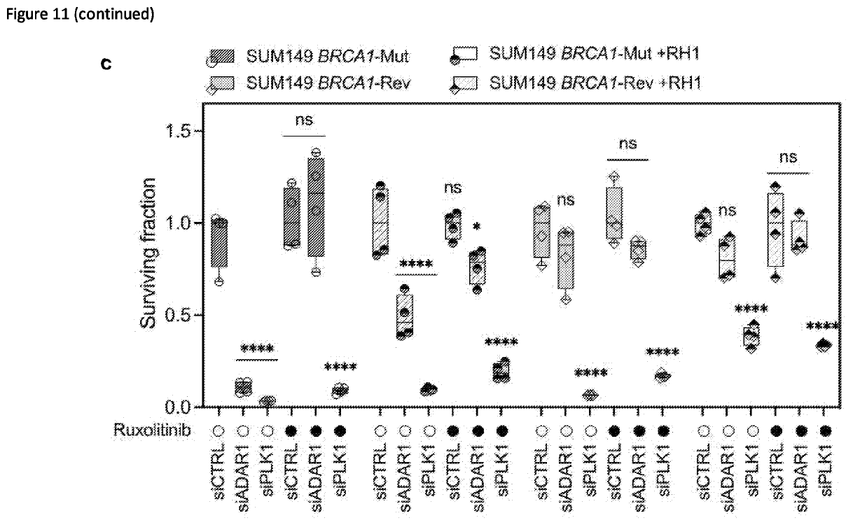

To evaluate the possibility that RNA sensors may modulate BRCA/ADAR1 synthetic lethality, the effects of ADAR1-targeting siRNA were assessed in the context of siRNA co-silencing of one of several RNA sensors in BRCA1-Mut and BRCA1-Rev SUM149 cells. These experiments indicated that the synthetic lethal effects of ADAR1 siRNA silencing were reversed upon RIG-1, LGP2, PKR or cGAS co-silencing (FIG. 11A, B), indicating that these sensors were individually required for BRCA/ADAR1 synthetic lethality. Of these, siRNA targeting of LGP2 and CGAS appeared to cause the greatest and most robust rescue effects (FIG. 11B; similar to that obtained with siRNA targeting of the type I interferon receptor IFNAR1).

To confirm the mechanistic basis of this observation, the effects of ADAR1-targeting siRNA were assessed in the context of pharmacological inhibition of the JAK/STAT pathway—a signalling pathway mediating cellular responses downstream of RNA sensors, in BRCA1-Mut and BRCA1-Rev SUM149 cells. It was demonstrated that exposure of BRCA1-Mut SUM149 cells to the JAK1/2 inhibitor ruxolitinib (a pharmacological inhibitor of the JAK/STAT signalling cascade) completely abrogated the synthetic lethal effects of ADAR1 siRNA silencing (FIG. 11C). It was further noted that whereas RNase H1 overexpression partially reversed the cell viability effect of ADAR1 siRNA silencing in BRCA1-Mut SUM149 cells, exposure to the JAK1/2 inhibitor ruxolitinib led to a complete rescue of ADAR1-dependent synthetic lethal effects (FIG. 11C), supporting the conclusion that JAK/STAT signalling activation mediated BRCA/ADAR1 synthetic lethality.

### Example 11—Selective siRNA Targeting of ADAR1p150 Uncovers BRCA/ADAR1p150 Synthetic Lethality

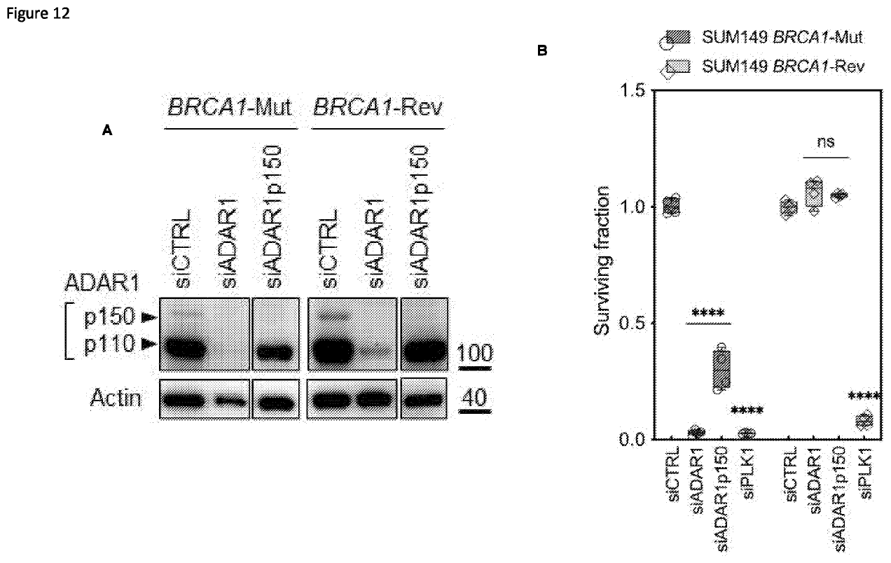

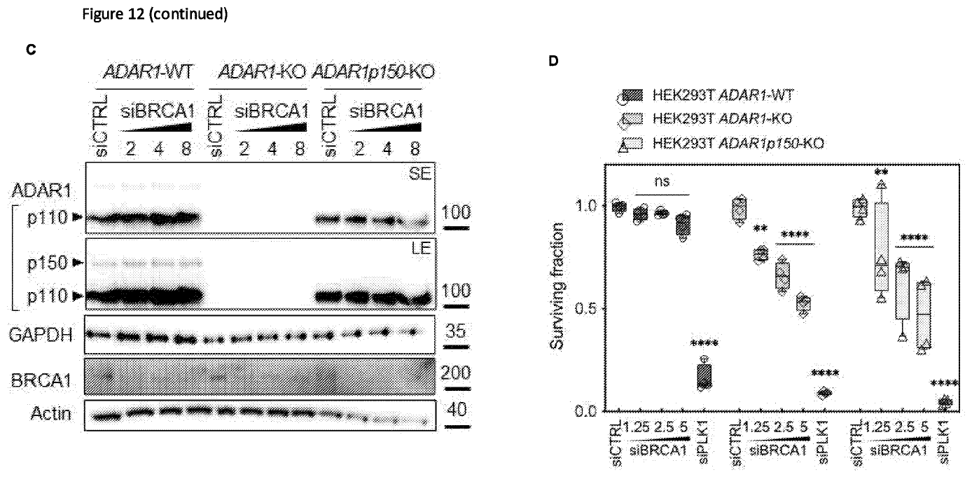

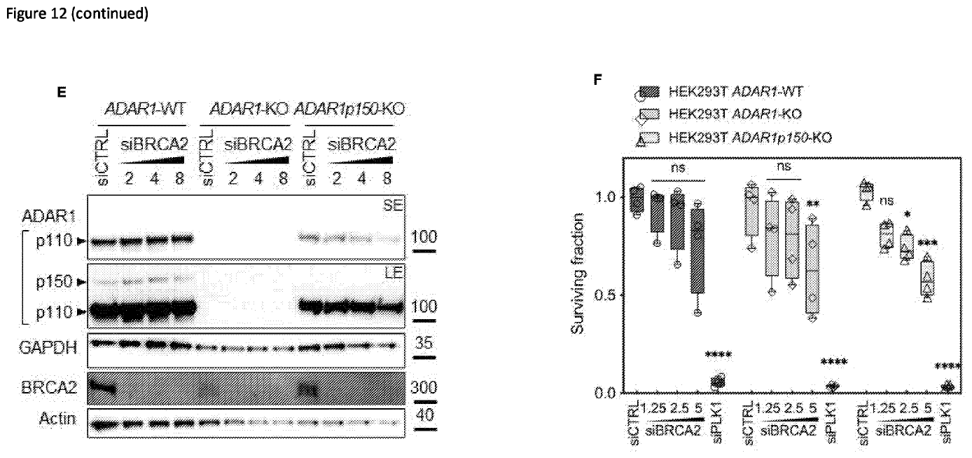

To investigate the relative contribution of ADAR1 isoforms to BRCA/ADAR1 synthetic lethality, the effects of ADAR1p150-selective siRNAs were compared to those of non-selective ADAR1-targeting siRNA in BRCA1-Mut and BRCA1-Rev SUM149 cells. As expected, selective siRNA silencing of ADAR1p150 completely silenced the expression of ADAR1p150 isoform while having minimal effects on the expression of ADAR1p110 isoform (FIG. 12A). This experiment further indicated that ADAR1p150-selective siRNA silencing reduced cell viability of BRCA1-Mut but not BRCA1-Rev SUM149 cells, to an extent similar to that of non-selective ADAR1 siRNA silencing (FIG. 12B).

To confirm the genetic basis of BRCA/ADAR1p150 synthetic lethality, its reciprocity was tested by assessing the effects of BRCA1- or BRCA2-targeting siRNAs in an extended model of ADAR1 deficiency (Chung et al. (2018) Cell 172(4):811-824) consisting of isogenic ADAR1-wildtype, ADAR1-mutant and ADAR1p150-mutant HEK293T cells (shown as HEK293T ADAR1-WT, HEK293T ADAR1-KO and HEK293T ADAR1p150-KO cells in FIG. 12, respectively). The BRCA/ADAR1p150 synthetic lethal effect was found to operate in such reciprocal setting, as BRCA1 or BRCA2 RNA interference elicited synthetic lethality in ADAR1p150-KO cells to the same extent as in ADAR1-KO cells, but not in ADAR1-WT cells (FIG. 12C-F).

1. An adenosine deaminase 1 (ADAR1) inhibitor for use in a method of treating an individual with a homologous recombination defective (HRD) cancer.

2. The ADAR1 inhibitor for use according to claim 1, wherein the homologous recombination deficiency is associated with a mutation and/or deficiency in one or more gene associated with HRD, optionally wherein said one or more gene associated with HRD is selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ.

3. The ADAR1 inhibitor for use according to claim 1 or 2, wherein the homologous recombination deficiency is associated with a mutation and/or deficiency in BRCA1 and/or BRCA2, preferably BRCA1.

4. The ADAR1 inhibitor for use according to any one of the preceding claims, wherein the cancer is breast cancer, ovarian cancer, pancreatic cancer, biliary tract cancer or prostate cancer.

5. The ADAR1 inhibitor for use according to any one of the preceding claims, wherein:
(a) the cancer is BRCA-mutated;
(b) the cancer is PARP inhibitor sensitive; and/or
(c) the breast cancer is BRCA-mutated and HER2-negative.

6. The ADAR1 inhibitor for use according to any one of the preceding claims, said method comprising:
(a) determining in a sample obtained from the individual whether the cancer is an HRD cancer; and
(b) administering a therapeutically effective amount of an ADAR1 inhibitor to the individual with HRD cancer.

7. The ADAR1 inhibitor for use according to claim 6, wherein determining whether the cancer is an HRD cancer comprises:
(a) determining the presence of a deficiency and/or mutation in one or more gene associated with homologous recombination deficiency, optionally a deficiency and/or mutation in one or more gene selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ;
(b) use of a companion diagnostic for homologous recombination deficiency, optionally which determines and/or quantifies loss of heterozygosity (LOH), telomeric allelic imbalance (LAI) and/or large-scale state transitions (LST), or any combination thereof;
(c) detecting and/or quantifying RAD51 foci within the sample, wherein reduced RAD51 signal is associated with homologous recombination deficiency;
(d) detecting and/or quantifying a homologous recombination deficiency defect, optionally a transcriptomic signature and/or mutational scar associated with homologous recombination deficiency; and/or
(e) detecting and/or quantifying platinum sensitivity of the cancer, wherein platinum sensitivity is associated with homologous recombination deficiency.

8. The ADAR1 inhibitor for use according to claim 6 or 7, wherein the step of determining the presence of a deficiency and/or mutation in one or more gene associated with homologous recombination deficiency is performed on nucleic acid sequences obtained from an individual's cancerous or noncancerous cells, optionally using direct sequencing, hybridisation to a probe, restriction fragment length polymorphism (RFLP) analysis, single-stranded conformation polymorphism (SSCP), PCR amplification of specific alleles, amplification of DNA target by PCR followed by a mini-sequencing assay, allelic discrimination during PCR, Genetic Bit Analysis, pyrosequencing, oligonucleotide ligation assay, analysis of melting curves, testing for a loss of heterozygosity (LOH) or next generation sequencing (NGS) techniques, single molecule sequencing techniques or nanostring nCounter technology.

9. The ADAR1 inhibitor for use according to any one of claims 6-8, wherein the step of determining the presence of a deficiency and/or mutation in one or more gene associated with homologous recombination deficiency comprises measuring protein expression of the one or more homologous recombination deficiency-associated gene in a sample obtained from the individual to determine whether the protein(s) is mutated or deficient, wherein optionally the step of determining protein expression of the one or more homologous recombination deficiency-associated gene comprises determining protein expression of the one or more homologous recombination deficiency-associated gene in the sample using one or more of immunohistochemistry, determining protein levels in a cell lysate by ELISA or Western blotting, and/or determining protein expression using a binding agent capable of specifically binding to a protein, or a fragment thereof.

10. The ADAR1 inhibitor for use in a method of treatment according to any one of the preceding claims, wherein the step of determining the expression of the one or more gene associated with homologous recombination deficiency comprises extracting RNA from a sample of cancer cells and measuring expression by real time PCR and/or by using a probe capable of hybridising to the RNA of one or more gene associated with homologous recombination deficiency, wherein optionally the probe is immobilised in a microarray.

11. The ADAR1 inhibitor for use in a method of treatment according to any one of the preceding claims, wherein the step of determining whether the individual has a cancer deficient in the one or more gene associated with homologous recombination deficiency:
(a) comprises or consists of identifying gene loss resulting from chromosomal instability through karyotype analysis of a sample obtained from the individual; and/or
(b) comprises or consists of identifying gene loss or pathogenic loss-of-function mutation detected in the individual's circulating tumour DNA or cell-free nucleic acids.

12. The ADAR1 inhibitor for use according to any one of the preceding claims, wherein:
(a) the ADAR1 inhibitor is a small molecule, a proteolysis-targeting chimeric molecule (PROTAC), a macrocyclic molecule, a molecular glue, a nucleic acid inhibitor, an antibody, an antibody-drug conjugate or a peptide; and/or
(b) the ADAR1 inhibitor is a small molecule is selected from 8-azaadenosine, 8-chloroadenosine, ZYS-1, 8-azanebularine, AVA-ADR-001 and rebecsinib.

13. The ADAR1 inhibitor for use according to any one of the preceding claims, wherein treatment with an ADAR1 inhibitor is combined with one or more further anti-cancer therapies.

14. The ADAR1 inhibitor for use in a method of treatment according to claim 13, wherein treatment with an ADAR1 inhibitor is used in conjunction with:
(a) one or more further chemotherapeutic agent(s); and/or
(b) one or more immunotherapeutic agent, which is optionally selected from immune checkpoint inhibitors, monoclonal antibodies, cytokines, cell therapy, cancer vaccines, oncolytic viruses, antisense oligodeoxynucleotides, antibody-drug conjugates and/or any modulator of the cytosolic nucleic acid sensing pathways.

15. The ADAR1 inhibitor for use in a method of treatment according to claim 13 or 14, wherein treatment with an ADAR1 inhibitor is used in conjunction with radiotherapy.

16. A method of selecting an individual having cancer for treatment with an adenosine deaminase 1 (ADAR1) inhibitor, the method comprising:
(a) determining in a sample obtained from the individual whether the cancer is an HRD cancer;
(b) selecting the individual for treatment with the ADAR1 inhibitor where the cancer is an HRD cancer; and
(c) providing an ADAR1 inhibitor suitable for administration to the individual;
wherein said method optionally further comprises administering a therapeutically effective amount of the ADAR1 inhibitor to the individual.

17. The method according to claim 16, wherein determining whether the cancer is an HRD cancer comprises:
(a) determining whether the cancer is mutated or deficient in one or more gene associated with homologous recombination deficiency, optionally a deficiency and/or mutation in one or more gene selected from BRCA1, BRCA2, ATM, BARD1, PALB2, BRIP1, RAD51B, RAD51C, RAD51D, CDK12, FAAP20, CHEK2, FAN1, FANCE, FANCM, and POLQ;
(b) use of a companion diagnostic for homologous recombination deficiency, optionally which determines and/or quantifies loss of heterozygosity (LOH), telomeric allelic imbalance (LAI) and/or large-scale state transitions (LST), or any combination thereof;
(c) detecting and/or quantifying RAD51 foci within the sample, wherein reduced RAD51 signal is associated with homologous recombination deficiency;
(d) detecting and/or quantifying a homologous recombination deficiency defect, optionally a transcriptomic signature and/or mutational scar associated with homologous recombination deficiency; and/or
(e) detecting and/or quantifying platinum sensitivity of the cancer, wherein platinum sensitivity is associated with homologous recombination deficiency.

18. The method according to claim 16 or 17, wherein:
(a) the one or more gene associated with homologous recombination deficiency is BRCA1 and/or BRCA2, preferably BRCA1;
(b) the cancer is breast cancer, ovarian cancer, pancreatic cancer, biliary tract cancer or prostate cancer; and/or
(c) the cancer is (i) BRCA-mutated; (ii) PARP inhibitor sensitive; and/or (iii) breast cancer which is BRCA-mutated and HER2-negative.

19. The method according to any one of claims 16-18, wherein the step of determining the presence of a deficiency and/or mutation in one or more gene associated with homologous recombination deficiency is performed on nucleic acid sequences obtained from an individual's cancerous or noncancerous cells, optionally using direct sequencing, hybridisation to a probe, restriction fragment length polymorphism (RFLP) analysis, single-stranded conformation polymorphism (SSCP), PCR amplification of specific alleles, amplification of DNA target by PCR followed by a mini-sequencing assay, allelic discrimination during PCR, Genetic Bit Analysis, pyrosequencing, oligonucleotide ligation assay, analysis of melting curves, testing for a loss of heterozygosity (LOH) or next generation sequencing (NGS) techniques, single molecule sequencing techniques or nanostring nCounter technology.

20. The method according to any one of claims 16-19, wherein the step of determining the presence of a deficiency and/or mutation in one or more gene associated with homologous recombination deficiency comprises measuring protein expression of the one or more homologous recombination deficiency-associated gene in a sample obtained from the individual to determine whether the protein(s) is mutated or deficient,
wherein optionally the step of determining protein expression of the one or more homologous recombination deficiency-associated gene comprises determining protein expression of the one or more homologous recombination deficiency-associated gene in the sample using one or more of immunohistochemistry, determining protein levels in a cell lysate by ELISA or Western blotting, and/or determining protein expression using a binding agent capable of specifically binding to a protein, or a fragment thereof.

21. The method according to any one of claims 16-20, wherein the step of determining the expression of the one or more gene associated with homologous recombination deficiency comprises extracting RNA from a sample of cancer cells and measuring expression by real time PCR and/or by using a probe capable of hybridising to the RNA of the one or more gene associated with homologous recombination deficiency, wherein optionally the probe is immobilised in a microarray.

22. The method according to any one of claims 16-21, wherein the step of determining whether the individual has a cancer deficient in the one or more gene associated with homologous recombination deficiency:
(a) comprises or consists of identifying gene loss resulting from chromosomal instability through karyotype analysis of a sample obtained from the individual; and/or
(b) comprises or consists of identifying gene loss or pathogenic loss-of-function mutation detected in the individual's circulating tumour DNA or cell-free nucleic acids.

23. The method according to any one of claims 16-22, wherein:
(a) the ADAR1 inhibitor is a small molecule, a proteolysis-targeting chimeric molecule (PROTAC), a macrocyclic molecule, a molecular glue, a nucleic acid inhibitor, an antibody, an antibody-drug conjugate, or a peptide; and/or
(b) the ADAR1 inhibitor is a small molecule is selected from 8-azaadenosine, 8-chloroadenosine, ZYS-1, 8-azanebularine, AVA-ADR-001 and rebecsinib.

24. The method according to any one of claims 16-23, wherein treatment with an ADAR1 inhibitor is combined with one or more further anti-cancer therapies.

25. The method according to claim 24, wherein treatment with an ADAR1 inhibitor is used in conjunction with:
(a) one or more further chemotherapeutic agent(s); and/or
(b) one or more immunotherapeutic agent, which is optionally selected from an immune checkpoint inhibitor, a monoclonal antibody, a cytokine, a cell therapy, a cancer vaccine, an oncolytic virus, an antisense oligodeoxynucleotide, an antibody-drug conjugate and/or any modulator of the cytosolic nucleic acid sensing pathways.

26. The method according to claim 24 or 25, wherein treatment with an ADAR1 inhibitor is used in conjunction with radiotherapy.
